(19) United States
(12) Patent Application Publication
(10) Pub. No.: US 2025/0257075 A1
(43) Pub. Date: Aug. 14, 2025

(54) MACROCYCLIC INHIBITORS OF KRAS FOR THE TREATMENT OF CANCER

(71) Applicant: Hoffmann-La Roche Inc., Little Falls, NJ (US)

(72) Inventors: Jianguo CHEN, Shanghai (CN); Haixia LIU, Shanghai (CN); Hong SHEN, Shanghai (CN); Weixing ZHANG, Shanghai (CN); Wei ZHU, Shanghai (CN)

(73) Assignee: Hoffmann-La Roche Inc., Little Falls, NJ (US)

(21) Appl. No.: 18/987,316

(22) Filed: Dec. 19, 2024

Related U.S. Application Data
(63) Continuation of application No. PCT/EP2023/068154, filed on Jul. 3, 2023.

(30) Foreign Application Priority Data
Jul. 4, 2022 (WO) PCT/CN2022/103694
Oct. 11, 2022 (WO) PCT/CN2022/124638
Jan. 5, 2023 (WO) PCT/CN2023/070765
Apr. 11, 2023 (WO) PCT/CN2023/087633

(51) Int. Cl. C07D 513/22; A61K 31/504; A61K 31/506; A61K 31/5377; A61P 35/00

(52) U.S. Cl. CPC C07D 513/22; A61K 31/504; A61K 31/506; A61K 31/5377; A61P 35/00

(57) ABSTRACT

The present invention relates to compounds of formula (I),

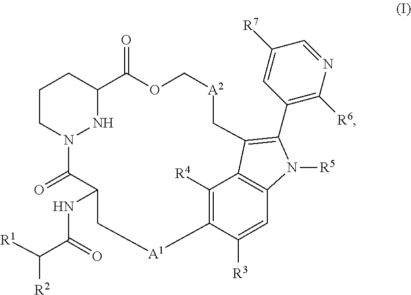

wherein R¹ to R⁷, A¹ and A² are as described herein, and their pharmaceutically acceptable salt thereof, and compositions including the compounds and methods of using the compounds.

### CROSS REFERENCE TO RELATED APPLICATIONS

This application is a continuation of International Application No. PCT/EP2023/068154, filed Jul. 3, 2023, which claims priority to CN Application No. PCT/CN2023/087633, filed Apr. 11, 2023, CN Application No. PCT/CN2023/070765, filed Jan. 5, 2023, CN Application No. PCT/CN2022/124638, filed Oct. 11, 2022, and CN Application No. PCT/CN2022/103694, filed Jul. 4, 2022, the disclosures of each of which are incorporated herein by reference in their entirety.

The present invention relates to organic compounds useful for therapy and/or prophylaxis in a mammal, and in particular to inhibition of KRAS G12C useful for treating cancers.

### FIELD OF THE INVENTION

RAS is one of the most well-known proto-oncogenes. Approximately 30% of human cancers contain mutations in three most notable members, KRAS, HRAS, and NRAS, making them the most prevalent oncogenic drivers. KRAS mutations are generally associated with poor prognosis especially in colorectal cancer, pancreatic cancer, lung cancers. As the most frequently mutated RAS isoform, KRAS has been intensively studied in the past years. Among the most commonly occurring KRAS alleles (including G12D, G12V, G12C, G13D, G12R, G12A, G12S, Q61H, etc), G12C, G12D, G12V represent more than half of all K-RAS-driven cancers across colorectal cancer (CRC), pancreatic ductal adenocarcinoma (PDAC), lung adenocarcinoma (LUAD). Of note, KRAS wild-type amplifications are also found in around 7% of all KRAS-altered cancers (ovarian, esophagogastric, uterine), ranking among the top alterations.

All RAS proteins belong to a protein family of small GTPases that hydrolyze GTP to GDP. KRAS is structurally divided into an effector binding lobe followed by the allosteric lobe and a carboxy-terminal region that is responsible for membrane anchoring. The effector lobe comprises the P-loop, switch I, and switch II regions. The switch I/II loops play a critical role in KRAS downstream signaling through mediating protein-protein interactions with effector proteins that include RAF in the mitogen-activated protein kinase (MAPK) pathway or PI3K in the phosphatidylinositol 3-kinase (PI3K)/protein kinase B (AKT) pathway.

KRAS protein switches between an inactive to an active form via binding to GTP and GDP, respectively. Under physiological conditions, the transition between these two states is regulated by guanine nucleotide exchange factors (GEFs), such as Son Of Sevenless Homolog 1 (SOS1), or GTPase-activating proteins (GAPs) that involve catalyzing the exchange of GDP for GTP, potentiating intrinsic GTPase activity or accelerating RAS-mediated GTP hydrolysis. In response to extracellular stimuli, the inactive RAS-GDP is converted to active RAS-GTP which directly binds to RAF RAS binding domains (RAF^{RBD}), recruiting RAF kinase family from cytoplasm to membranes, where they dimerize and become active. The activated RAF subsequently carries out a chain of phosphorylation reactions to its downstream Mitogen-activated protein kinase (MEK) and extracellular signal-regulated kinase (ERK), and propagates the growth signal. Of the RAF family of protein kinases (three known isoforms ARAF, BRAF, CRAF/RAF1), BRAF is most frequently mutated and remains the most potent activator of MEK. Despite that individual RAS and RAF family members revealed distinct binding preferences, all RAFs possess the conserved RBD for forward transmission of MAPK singnaling, frequently used for characterize KRAS inhibition (e.g. KRAS-BRAF^{RBD} herein). For KRAS, mutations at positions 12, 13, 61, and 146 lead to a shift toward the active KRAS form through impairing nucleotide hydrolysis or activating nucleotide exchange, leading to hyper-activation of the MAPK pathway that results in tumorigenesis.

Despite its well-recognized importance in cancer malignancy, continuous efforts in the past failed to develop approved therapies for KRAS mutant cancer until recently, the first selective drug AMG510 has fast approval as second line treatment in KRAS G12C driven non-small cell lung cancer (NSCLC). Nevertheless, the clinical acquired resistance to KRAS G12C inhibitors emerge rigorously with disease progresses after around 6 month of treatment. All of the mutations converge to reactivate RAS-MAPK signaling, with secondary RAS mutants at oncogenic hotspots (e.g. G12/G13/Q61) and within the switch II pocket (e.g. H95, R68, and Y96) have been observed; moreover, over 85% of all KRAS-mutated or wild-type amplified driven cancers still lack novel agents. Altogether, both the myriad of escape mechanism and various oncogenic alleles, highlight the urgent medical need for additional KRAS therapies. As such, we invented oral compounds that target and inhibit KRAS alleles for the treatment of KRAS mutant driven cancers.

First generation KRAS G12C inhibitors like Sotorosib, Adagrasib targeting on ‘GDP bound off’ form (RASOFF) of KRAS G12C mutation have demonstrated promising efficacy. While this treatment has benefited many patients with activating KRAS mutations, almost all who initially benefited will eventually acquire resistance via various mechanism. Increasing cases of KRAS G12C second mutations have been identified either from patients' samples such as Y96D, R68S, H95D, H95Q, H95R, V8L (Tanaka et al., Cancer Discovery (2021), Awad et al., NEJM (2021), Ho et al., EJC (2021), Zhao et al., Nature (2021), Tsai et al., JCI (2022)), or discovered from saturation mutagenesis (Siyu et al, PNAS (2022)) and ENU mutagenesis (Takamasa et al, J Thorac Oncol (2021)) that demonstrated resistance to KRAS(OFF) G12C inhibitors. Therefore, there are unmet needs to prevent the acquisition of one or more mutations in RAS that confer resistance to the RAS(OFF) inhibitor.

### SUMMARY OF THE INVENTION

The present invention relates to novel compounds of formula (I),

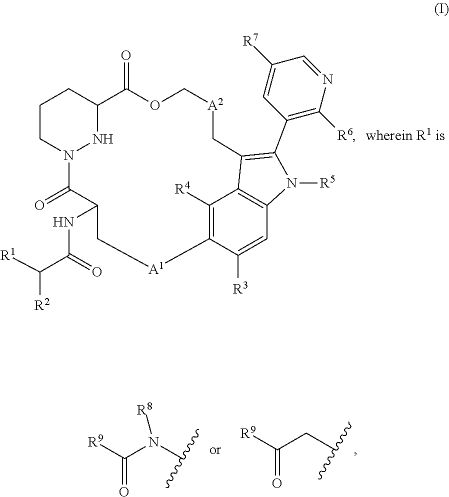

wherein R⁸ is C₁₋₆alkyl;
   R⁹ is C₃₋₇cycloalkyl, azetidinyl or phenyl, said C₃₋₇cycloalkyl, azetidinyl and phenyl being substituted by haloC₃₋₆alkynyl, (haloC₃₋₆alkylpyrimidinyl)C₂₋₆alkynyl or pyrimidinylC₂₋₆alkynyl;
R² is C₁₋₆alkyl;
R³ is H or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene;
A² is C₁₋₆alkylene;
with the proviso that R³ and R⁴ are not H simultaneously;
or a pharmaceutically acceptable salt thereof.

The invention also relates to their manufacture, medicaments based on a compound in accordance with the invention and their production as well as the use of compounds of formula (I) or (Ia) thereof as inhibitor of KRAS.

The compound of current invention addressed GSH toxicity issue comparing with the reference compounds. The compounds of formula (I) or (Ia) show good KRAS inhibition for G12C, G12D and G12V. In another embodiment, the compounds of this invention showed superior cancer cell inhibition and human hepatocyte stability. In addition, the compounds of formula (I) or (Ia) also show good or improved cytotoxicity, solubility, and single dose pharmacokinetics (SDPK) profiles. Furthermore, the compound of current invention demonstrated good efficacy towards a second mutation as mentioned in this application.

### BRIEF DESCRIPTION OF THE FIGURE

FIG. 1: X-ray crystallographic analysis of compound G5.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkoxy” denotes C₁₋₆alkyl-O—.

The term “C₁₋₆alkylene” denotes a linear or branched saturated divalent hydrocarbon group of 1 to 6 carbon atoms or a divalent branched saturated divalent hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “halogen” and “halo” are used interchangeably herein and denote fluoro, chloro, bromo, or iodo.

The term “C₂₋₆alkynyl” denotes a monovalent linear or branched hydrocarbon group of 2 to 6 carbon atoms with at least one triple bond. In particular embodiments, alkynyl has 2 to 4 carbon atoms with at least one triple bond. Examples of C₂₋₆alkynyl include ethynyl (—C═CH), prop-1-ynyl (—C═CCH₃), prop-2-ynyl (propargyl, —CH₂C═CH), but-1-ynyl, but-2-ynyl, and but-3-ynyl.

The term “halogen” and “halo” are used interchangeably herein and denote fluoro, chloro, bromo, or iodo.

The term “C₃₋₆alkynyl” denotes a monovalent linear or branched hydrocarbon group of 3 to 6 carbon atoms with at least one triple bond. In particular embodiments, alkynyl has 3 to 4 carbon atoms with at least one triple bond. Examples of C₃₋₆alkynyl include prop-1-ynyl (—C═CCH₃), prop-2-ynyl (propargyl, —CH₂C═CH), but-1-ynyl, but-2-ynyl, and but-3-ynyl.

The term “haloC₁₋₆alkyl” denotes a C₁₋₆alkyl group wherein at least one of the hydrogen atoms of the C₁₋₆alkyl group has been replaced by same or different halogen atoms, particularly fluoro atoms. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, or trifluoromethyl.

The term “haloC₃₋₆alkynyl” denotes a C₃₋₆alkynyl group wherein at least one of the hydrogen atoms of the C₃₋₆alkynyl group have been replaced by same or different halogen atoms. Examples of haloC₃₋₆alkynyl include 3,3,3-trifluoroprop-1-ynyl.

The term “C₃₋₇cycloalkyl” denotes a monovalent saturated monocyclic or bicyclic hydrocarbon group of 3 to 7 ring carbon atoms. Bicyclic means consisting of two saturated carbocycles having one or more carbon atoms in common. Examples for monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, bicyclo[1.1.1]pentanyl, or bicyclo[2.2.2]octanyl.

The term “thiazolylene” denotes a divalent thiazolyl group.

The term “oxo” denotes a divalent oxygen atom ═O.

The term “dimethylmethylene” denotes

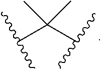 [SMILES: *C(*)(C)C]

The term “protecting group” denotes the group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups.

The skilled of the art would understand that the following structures of compounds of formula (Ia) and (Ia′) are equal especially for the chiral centers:

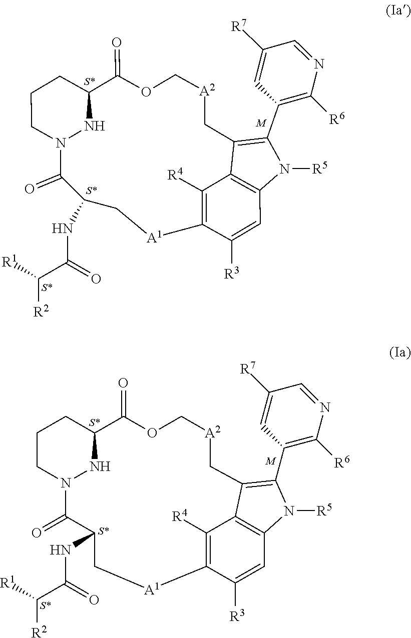

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### Inhibitor of KRAS

The present invention relates to (i′) a compound of formula (I),

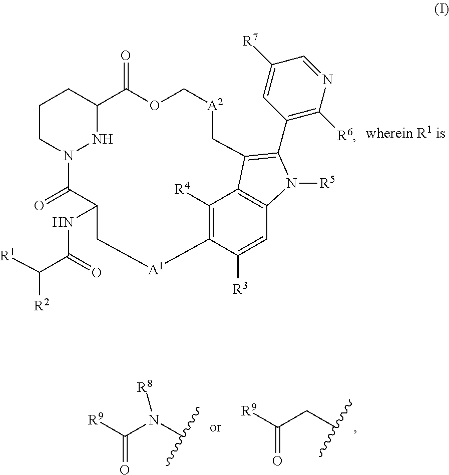

wherein R⁸ is C₁₋₆alkyl;
   R⁹ is C₃₋₇cycloalkyl, azetidinyl or phenyl, said C₃₋₇cycloalkyl, azetidinyl and phenyl being substituted by haloC₃₋₆alkynyl, (haloC₃₋₆alkylpyrimidinyl)C₂₋₆alkynyl or pyrimidinylC₂₋₆alkynyl;
R² is C₁₋₆alkyl;
R³ is H or halogen;
R w is H or halogen;
R⁵ is C₆alkyl or haloC₁₋₆alkyl;
R⁶ is C₁₋₆alkoxyC₆alkyl;
R⁷ is morpholinyl, (haloC₁₋₆alkyl)piperazinyl or C₁₋₆alkylpiperazinyl;
A¹ is thiazolylene;
A² is C₁₋₆alkylene;
with the proviso that R³ and R⁴ are not H simultaneously;
or a pharmaceutically acceptable salt thereof.

Another embodiment of present invention is (ii′) a compound of formula (Ia),

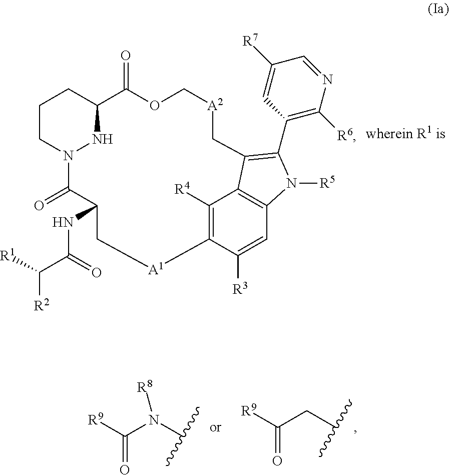

wherein R⁸ is C₁₋₆alkyl;
   R⁹ is C₃₋₇cycloalkyl, azetidinyl or phenyl, said C₃₋₇cycloalkyl, azetidinyl and phenyl being substituted by haloC₃₋₆alkynyl, (haloC₃₋₆alkylpyrimidinyl)C₂₋₆alkynyl or pyrimidinylC₂₋₆alkynyl;
R² is C₁₋₆alkyl;
R³ is H or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene;
A² is C₁₋₆alkylene;
with the proviso that R³ and R⁴ are not H simultaneously;
or a pharmaceutically acceptable salt thereof.

The present invention relates to (i) a compound of formula (I),

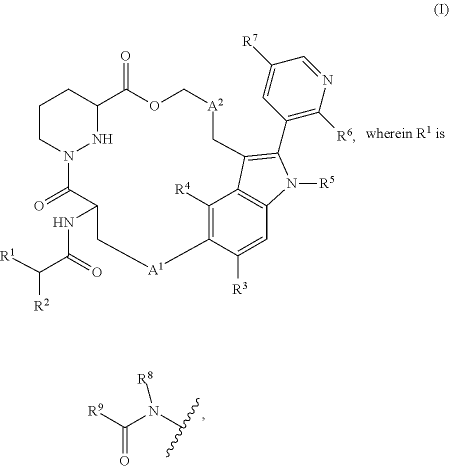

wherein R⁸ is C₁₋₆alkyl;
   R⁹ is C₃₋₇cycloalkyl, azetidinyl or phenyl, said C₃₋₇cycloalkyl, azetidinyl and phenyl being substituted by haloC₃₋₆alkynyl or pyrimidinylC₂₋₆alkynyl;
R² is C₁₋₆alkyl;
R³ is H or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene;
A² is C₁₋₆alkylene;
with the proviso that R³ and R⁴ are not H simultaneously;
or a pharmaceutically acceptable salt thereof.

Another embodiment of present invention is (ii) a compound of formula (Ia),

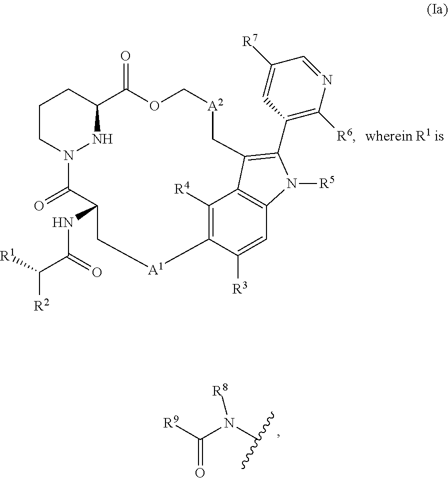

wherein R⁸ is C₁₋₆alkyl;
   R⁹ is C₃₋₇cycloalkyl, azetidinyl or phenyl, said C₃₋₇cycloalkyl, azetidinyl and phenyl being substituted by haloC₃₋₆alkynyl or pyrimidinylC₂₋₆alkynyl;
R² is C₁₋₆alkyl;
R³ is H or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene;
A² is C₁₋₆alkylene;
with the proviso that R³ and R⁴ are not H simultaneously;
or a pharmaceutically acceptable salt thereof.

A further embodiment of present invention is (iii) a compound of formula (I) or (Ia) according to (i), (ii), (i′) or (ii′), or a pharmaceutically acceptable salt thereof, wherein R¹ is

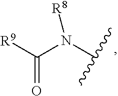

wherein R⁸ is C₁₋₆alkyl; R⁹ is C₃₋₇cycloalkyl substituted by haloC₃₋₆alkynyl.

A further embodiment of present invention is (iv) a compound of formula (I) or (Ia), according to any one of (i) to (iii), (i′) and (ii′), or a pharmaceutically acceptable salt thereof, wherein R¹ is

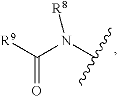

wherein R⁸ is methyl; R⁹ is cyclobutyl substituted by 3,3,3-trifluoroprop-1-ynyl.

A further embodiment of present invention is (v) a compound of formula (I) or (Ia) according to any one of (i) to (iv), (i′) and (ii′), wherein R⁹ is 3-(3,3,3-trifluoroprop-1-ynyl)cyclobutyl.

A further embodiment of present invention is (vi) a compound of formula (I) or (Ia), or a pharmaceutically acceptable salt thereof, according to any one of (i) to (v), (i′) and (ii′), wherein R² is isopropyl.

A further embodiment of present invention is (vii) a compound of formula (I) or (Ia), or a pharmaceutically acceptable salt thereof, according to any one of (i) to (vi), (i′) and (ii′), wherein R³ is halogen.

A further embodiment of present invention is (viii) a compound of formula (I) or (Ia), or a pharmaceutically acceptable salt thereof, according to any one of (i) to (vii), (i′) and (ii′), wherein R³ is fluoro.

A further embodiment of present invention is (ix) a compound of formula (I) or (Ia), or a pharmaceutically acceptable salt thereof, according to any one of (i) to (viii), (i′) and (ii′), wherein R⁴ is H or fluoro.

A further embodiment of present invention is (x) a compound of formula (I) or (Ia), or a pharmaceutically acceptable salt thereof, according to any one of (i) to (ix), (i′) and (ii′), wherein R⁴ is H.

A further embodiment of present invention is (xi) a compound of formula (I) or (Ia), or a pharmaceutically acceptable salt thereof, according to any one of (i) to (x), (i′) and (ii′), wherein R⁵ is ethyl or 2,2,2-trifluoroethyl.

A further embodiment of present invention is (xii) a compound of formula (I) or (Ia), or a pharmaceutically acceptable salt thereof, according to any one of (i) to (xi), (i′) and (ii′), wherein R⁶ is 1-methoxyethyl.

A further embodiment of present invention is (xiii) a compound of formula (I) or (Ia), or a pharmaceutically acceptable salt thereof, according to any one of (i) to (xii), (i′) and (ii′), wherein R⁷ is morpholinyl, 4-(2,2,2-trifluoroethyl)piperazin-1-yl or 4-methylpiperazin-1-yl.

A further embodiment of present invention is (xiv) a compound of formula (I) or (Ia), or a pharmaceutically acceptable salt thereof, according to any one of (i) to (xiii), (i′) and (ii′), wherein A¹ is

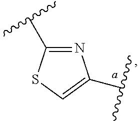 [SMILES: *c1csc(*)n1]

herein bond “a” connects to indole ring.

A further embodiment of present invention is (xv) a compound of formula (I) or (Ia), or a pharmaceutically acceptable salt thereof, according to any one of (i) to (xiv), (i′) and (ii′), wherein A² is dimethylmethylene.

Another embodiment of present invention is (xvi) a compound of formula (I) or (Ia), according to (i) or (ii), (i′) or (ii′), wherein
R¹ is

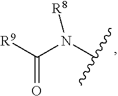

wherein R⁸ is C₁₋₆alkyl; R⁹ is C₃₋₇cycloalkyl substituted by haloC₃₋₆alkynyl;
R² is C₁₋₆alkyl;
R³ is halogen;
R⁴ is H;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is

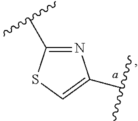 [SMILES: *c1csc(*)n1]

wherein bond “a” connects to indole ring;
A² is C₁₋₆alkylene;
or a pharmaceutically acceptable salt thereof.

Another embodiment of present invention is (xvii) a compound of formula (I) or (Ia), according to (xvi), wherein
R¹ is

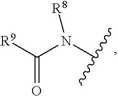

wherein R⁸ is methyl; R⁹ is 3-(3,3,3-trifluoroprop-1-ynyl)cyclobutyl;
R² is isopropyl;
R³ is fluoro;
R4 is H;
R⁵ is ethyl or 2,2,2-trifluoroethyl;
R⁶ is (1S)-1-methoxyethyl;
R⁷ is morpholinyl, 4-(2,2,2-trifluoroethyl)piperazin-1-yl or 4-methylpiperazin-1-yl;
A¹ is

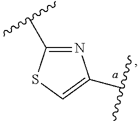 [SMILES: *c1csc(*)n1]

wherein bond “a” connects to indole ring;
A² is dimethylmethylene;
or a pharmaceutically acceptable salt thereof.

Another embodiment of present invention is (xviii) a compound of formula (I) or (Ia) selected from the following:
trans-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide;
cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)benzamide;
cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(2-pyrimidin-2-ylethynyl)cyclobutanecarboxamide;
cis-N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
cis-N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
cis-N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
trans-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)cyclohexanecarboxamide;
cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)cyclohexanecarboxamide;
cis-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
cis-N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-[2-[5-(trifluoromethyl)pyrimidin-2-yl]ethynyl]cyclobutanecarboxamide;
N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide;
N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide;
cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-[2-[4-(trifluoromethyl)pyrimidin-2-yl]ethynyl]cyclobutanecarboxamide;
N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide;
(2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-isopropyl-4-oxo-4-[3-(3,3,3-trifluoroprop-1-ynyl)azetidin-1-yl]butanamide;
N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide;
cis-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-[2-[4-(trifluoromethyl)pyrimidin-2-yl]ethynyl]cyclobutanecarboxamide; and
cis-N-[(1S)-1-[[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
   or a pharmaceutically acceptable salt thereof.

Another embodiment of present invention is related to (xix) a process for the preparation of a compound according to any one of (i) to (xviii) comprising the following step:
a) coupling reaction between compound of formula (II),

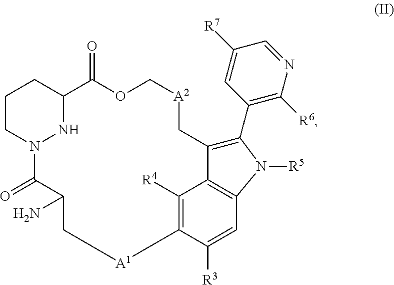

and acid (III),

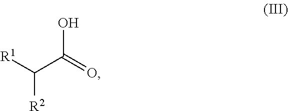

in the presence of a coupling reagent and a base to form the compound of formula (I);
wherein R¹, R², R³, R⁴, R⁵, R⁶, R⁷, A¹ and A² are defined as in any one of (i) to (xvii); the coupling reagent is T₃P, HATU, PyBOP or EDCI/HOBt; the base is TEA, DIEPA or DMAP.

Another embodiment of present invention is (xx) a compound or pharmaceutically acceptable salt according to any one of (i) to (xviii), (i′) and (ii′), for use as therapeutically active substance.

Another embodiment of present invention is (xxi) a pharmaceutical composition comprising a compound in accordance with any one of (i) to (xviii), (i′) and (ii′), and a pharmaceutically acceptable excipient.

Another embodiment of present invention is (xxii) the use of a compound according to any one of (i) to (xviii), (i′) and (ii′), for treating a KRAS G12C protein-related disease.

Another embodiment of present invention is (xxiii) the use of a compound according to any one of (i) to (xviii) for treating a KRAS G12C, G12D and G12V protein-related disease.

Another embodiment of present invention is (xxiv) the use of a compound according to any one of (i) to (xviii), (i′) and (ii′), for inhibiting RAS interaction with downstream effectors, wherein the downstream effectors are RAF and PI3K.

Another embodiment of present invention is (xxv) the use of a compound according to any one of (i) to (xviii), (i′) and (ii′), for inhibiting the propagating oncogenic MAPK and PI3K signaling.

Another embodiment of present invention is (xxvi) the use of a compound according to any one of (i) to (xviii), (i′) and (ii′), for the treatment or prophylaxis of KRAS mutation driven cancers, wherein the cancer is selected from pancreatic cancer, colorectal cancer, lung cancer, esophageal cancer, gallbladder cancer, melanoma ovarian cancer and endometrial cancer.

Another embodiment of present invention is (xxvii) the use of a compound according to any one of (i) to (xviii), (i′) and (ii′), for the treatment or prophylaxis of KRAS mutation driven cancers, wherein the cancer is selected from pancreatic adenocarcinoma, colorectal cancer and non-small cell lung cancer.

Another embodiment of present invention is (xxviii) a compound or pharmaceutically acceptable salt according to any one of (i) to (xviii), (i′) and (ii′), for the treatment or prophylaxis of KRAS mutation driven cancers, wherein the cancer is selected from pancreatic adenocarcinoma, colorectal cancer and non-small cell lung cancer.

Another embodiment of present invention is (xxix) the use of a compound according to any one of (i) to (xviii), (i′) and (ii′), for the treatment or prophylaxis of KRAS mutation driven cancers, wherein the cancer comprises a first mutation that is G12C, and a second mutation at a position selected from V8A, V9Y, S17E, T58I, A59T, S65W, R68S, D69P, M72I, D92R, H95N, Y96D, Q99F, Q99W, Y96H, and F156L.

Another embodiment of present invention is (xxx) the use of a compound according to any one of (i) to (xviii), (i′) and (ii′), for the preparation of a medicament for the treatment or prophylaxis of KRAS mutation driven cancers, wherein the cancer is selected from pancreatic adenocarcinoma, colorectal cancer and non-small cell lung cancer.

Another embodiment of present invention is (xxxi) the use of a compound according to any one of (i) to (xviii), (i′) and (ii′), for the preparation of a medicament for the treatment or prophylaxis of KRAS mutation driven cancers, wherein the cancer comprises a first mutation that is G12C, and a second mutation at a position selected from V8A, V9Y, S17E, T58I, A59T, S65W, R68S, D69P, M72I, D92R, H95N, Y96D, Q99F, Q99W, Y96H, and F156L.

Another embodiment of present invention is (xxxii) a method for the treatment or prophylaxis of KRAS mutation driven cancers, wherein the cancer is selected from pancreatic adenocarcinoma, colorectal cancer and non-small cell lung cancer, which method comprises administering a therapeutically effective amount of a compound as defined in any one of (i) to (xviii), (i′) and (ii′).

Another embodiment of present invention is (xxxiii) a method for the treatment or prophylaxis of KRAS mutation driven cancers, wherein the cancer comprises a first mutation that is G12C, and a second mutation at a position selected from V8A, V9Y, S17E, T58I, A59T, S65W, R68S, D69P, M72I, D92R, H95N, Y96D, Q99F, Q99W, Y96H, and F156L.

Another embodiment of present invention is (xxxiv) a compound or pharmaceutically acceptable salt according to any one of (i) to (xviii), (i′) and (ii′), when manufactured according to a process of (xix).

### PHARMACEUTICAL COMPOSITIONS AND ADMINISTRATION

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit mutant RAS (e.g. KRAS G12C) interaction with RAF, blocking the oncogenic MAPK signaling. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 0.1 to 1000 mg/kg of patient body weight per day, with the typical initial range of compound used being 0.3 to 15 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 1 to 1000 mg of the compound of the invention compounded with about 1 to 1000 mg anhydrous lactose, about 1 to 1000 mg sodium croscarmellose, about 1 to 1000 mg polyvinylpyrrolidone (PVP) K30, and about 1 to 1000 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of mutant KRAS-driven cancers. Another embodiment includes a pharmaceutical composition comprising a compound of Formula (I) for use in the treatment of mutant KRAS-driven cancers.

The following composition A and B illustrate typical compositions of the present invention, but serve merely as representative thereof.

### Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention induce a new binding pocket in KRAS by driving formation of a high affinity tri-complex between KRAS protein and the widely expressed cyclophilin A (CYPA), which inhibit KRAS interaction with downstream effectors, such as RAF and PI3K. Accordingly, the compounds of the invention are useful for inhibiting the propagating oncogenic MAPK and PI3K signaling, reducing cell proliferation, in particular cancer cells. Compounds of the invention are useful for termination of RAS signaling in cells that express RAS mutant, e.g. KRAS mutation driven pancreatic cancer, colorectal cancer, lung cancer, esophageal cancer, gallbladder cancer, melanoma ovarian cancer, endometrial cancer, etc. Alternatively, compounds of the invention are useful for termination of RAS signaling in malignant solid tumor where the oncogenic role of KRAS mutation is reinforced by dysregulation or mutation of effector pathways as MAPK, PI3K-AKT-mTOR (Mammalian target of rapamycin) driven signaling, for targeted therapy in pancreatic adenocarcinoma, colorectal cancer, non-small cell lung cancer, etc.

Another embodiment includes a method of treating or preventing cancer in a mammal in need of such treatment, wherein the method comprises administering to said mammal a therapeutically effective amount of a compound of formula (I), a stereoisomer, tautomer, prodrug or pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R⁷, A¹ and A² are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

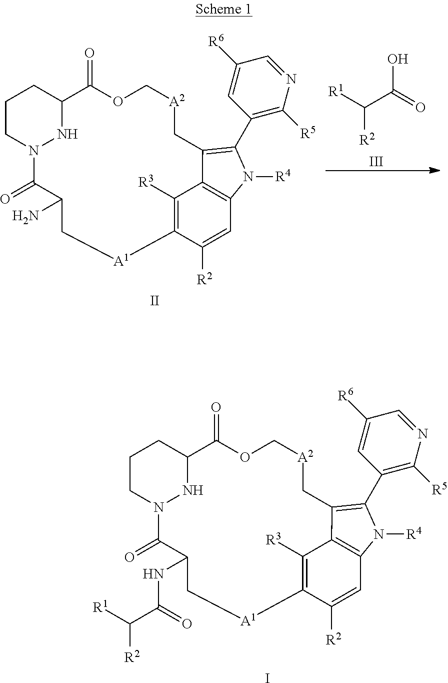

Compound of formula II was synthesized according to the procedure described in Intermediate A to K. Compound of formula (I) can be obtained by a coupling reaction between acid (III) and compound of formula (II) with coupling reagent(s), such as T₃P, HATU, PyBOP and EDCI/HOBt, in the presence of a base, such as TEA, DIEPA and DMAP.

Compounds of this invention can be obtained as mixtures of diastereomers or enantiomers, which can be separated by methods well known in the art, e.g. (chiral) HPLC or SFC. In another embodiment, compound of formula (I) can be obtained according to above scheme by using corresponding chiral starting materials.

This invention also relates to a process for the preparation of a compound of formula (I) comprising following step:
a) coupling reaction between compound of formula (II),

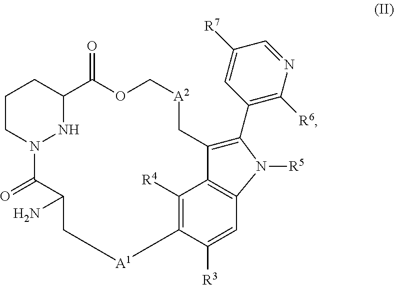

and acid (III),

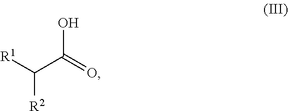

in the presence of a coupling reagent and a base to form the compound of formula (I); wherein
in step a) the coupling reagent can be, for example, T₃P, HATU, PyBOP or EDCI/HOBt; the base can be, for example, TEA, DIEPA or DMAP.

A compound of formula (I) or (Ia) when manufactured according to the above process is also an object of the invention.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:

| | |
|---|---|
| ACN | acetonitrile |
| aq. | Aqueous |
| Boc-N-Me-Val-OH | N-(tert-Butoxycarbonyl)-N-methyl-L-valine |
| (Boc)₂O | Di-tert-butyldicarbonate |
| (R)-binap | (R)-(+)-2,2′-Bis(diphenylphosphino)-1,1′-binaphthyl |
| CDCl₃: | deuterated chloroform |
| CD₃OD: | deuterated methanol |
| COMU | (1-Cyano-2-ethoxy-2-oxoethylidenaminooxy)dimethylamino-morpholino- |
| | carbenium hexafluorophosphate |
| DIEPA: | N,N-diethylpropylamine |
| DIBAL-H | Diisobutylaluminium hydride |
| DMAP: | 4-Dimethylaminopyridine |
| DMF: | dimethyl formamide |
| DMP | 1,1,1-Tris(acetyloxy)-1,1-dihydro-1,2-benziodoxol-3-(1H)-one |
| DMSO: | dimethyl sulfoxide |
| EDCI: | N-Ethyl-N′-(3-dimethylaminopropyl)carbodiimide hydrochloride |
| EtOAc or EA: | ethyl acetate |
| FRET | fluorescence resonance energy transfer |
| HATU: | (1-[Bis(dimethylamino)methylene]-1H-1,2,3-triazolo[4,5-b]pyridinium |
| | 3-oxid hexafluorophosphate) |
| hr(s): | hour(s) |
| HPLC: | high performance liquid chromatography |
| HOBt: | N-hydroxybenzotriazole |
| H-VAL-OTBU HCl | (S)-tert-Butyl 2-amino-3-methylbutanoate hydrochloride |
| [Ir(OMe)(COD)]₂ | (1,5-Cyclooctadiene)(methoxy)iridium(I) dimer |
| LDA | Lithium diisopropylamide |
| MS: (ESI): | mass spectroscopy (electron spray ionization) |
| min(s) | minute(s) |
| MTBE | Methyl tert-butyl ether |
| NMM | N-Methylmorpholine |
| NMR: | nuclear magnetic resonance |
| NMO | 4-Methylmorpholine N-oxide |
| obsd. | Observed |
| Pd(dppf)Cl₂ | [1,1′-Bis(diphenylphosphino)ferrocene]dichloropalladium(II) |
| Pd(dtbpf)Cl₂ | [1,1′-Bis(di-tert-butylphosphino)ferrocene]dichloropalladium(II) |
| prep-HPLC | preparative high performance liquid chromatography |
| PyBOP: | benzotriazol-1-yloxytripyrrolidinophosphonium hexafluorophosphate |
| RT or rt: | room temperature |
| sat. | saturated |
| SFC | supercritical fluid chromatography |
| TEA: | triethylamine |
| TFA: | trifluoroacetic acid |
| THF: | tetrahydrofuran |
| TEA: | trimethylamine |
| TMEDA | Tetramethylethylenediamine |
| TMSCF₃ | Trifluoromethyltrimethylsilane |
| T₃P: | propylphosphonic anhydride |

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃—H₂O in MeOH), back pressure 100 bar, detection UV@254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃H₂O in H₂O; B: acetonitrile;
Basic condition II: A: 0.025% NH₃H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

### Preparation of Intermediate

### Intermediate A

### 1-[6-[(1S)-1-methoxyethyl]-5-(4,4,5,5-tetramethyl-1,3,2-dioxaborolan-2-yl)-3-pyridyl]-4-methyl-piperazine

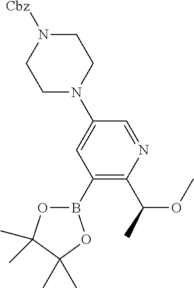

The title intermediate A was prepared according to the following scheme:

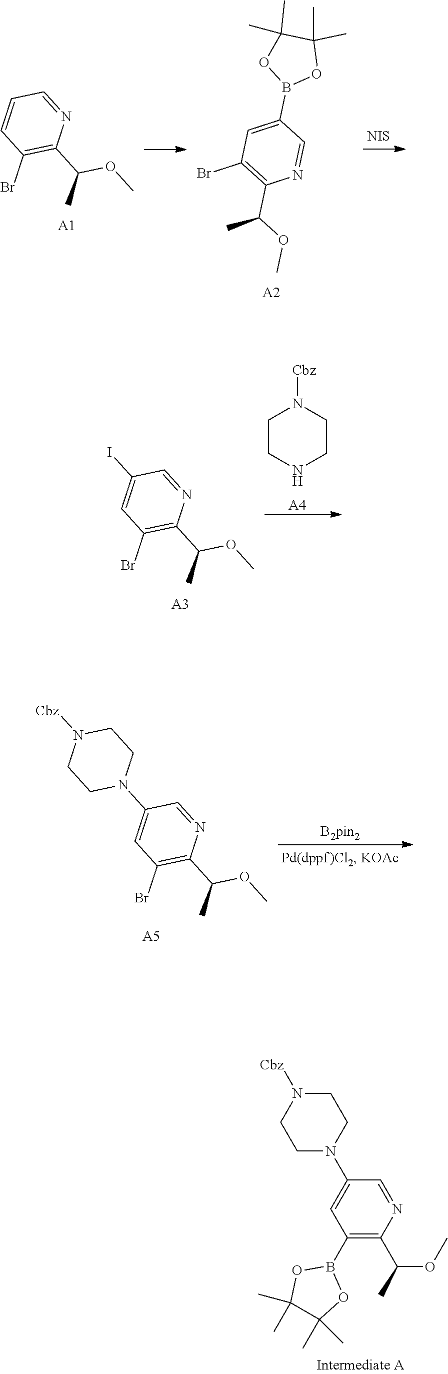

### Step 1: Preparation of 3-bromo-2-[(1S)-1-methoxyethyl]-5-(4,4,5,5-tetramethyl-1,3,2-dioxaborolan-2-yl)pyridine (compound A2)

To a solution of 3-bromo-2-[(1S)-1-methoxyethyl]pyridine (compound A1, 2.0 g, 9.26 mmol) and bis(pinacolato)diboron (3.5 g, 13.9 mmol) in THF (30 mL) were added 4,4′-di-tert-butyl-2,2′-bipyridin (372.7 mg, 1.39 mmol) and [Ir(OMe)(COD)]₂ (306.3 mg, 0.460 mmol). The mixture was stirred at 75° C. for 16 hours under N₂ protection. The mixture was filtrated and the filtrate was concentrated in vacuo. The residue was purified by silica gel chromatography (EA/PE: 0-20%) to afford 3-bromo-2-[(1S)-1-methoxyethyl]-5-(4,4,5,5-tetramethyl-1,3,2-dioxaborolan-2-yl)pyridine (compound A2, 2.4 g) as yellow oil. ¹H NMR (400 MHz, CDCl3) δ ppm 8.91 (d, J=1.4 Hz, 1H), 8.21 (d, J=1.4 Hz, 1H), 4.95 (q, J=6.5 Hz, 1H), 3.30 (s, 3H), 1.49 (d, J=6.5 Hz, 3H), 1.35 (s, 12H).

### Step 2: Preparation of 3-bromo-5-iodo-2-[(LS)-1-methoxyethyl]pyridine (compound A3)

To a solution of 3-bromo-2-[(1S)-1-methoxyethyl]-5-(4,4,5,5-tetramethyl-1,3,2-dioxaborolan-2-yl)pyridine (compound A2, 2.5 g, 7.3 mmol) in ACN (40 mL) was added N-iodosuccinimide (4.1 g, 18.27 mmol). The mixture was stirred at 90° C. for 40 hrs under N₂ protection. The reaction was quenched with saturated solution of Na₂SO₃ (40 mL) and the reaction mixture was extracted with EtOAc (30 mL, twice). The combined organic layer was washed with brine (50 mL), filtered and the filtrate was concentrated under vacuum. The residue was purified by silica gel chromatography (EA/PE: 0-20%) to afford 3-bromo-5-iodo-2-[(1S)-1-methoxyethyl]pyridine (compound A3, 660 mg) as yellow oil. MS calc'd 342 (MH⁺), measured 341.8 (MH⁺).

### Step 3: Preparation of benzyl 4-[5-bromo-6-[(1S)-1-methoxyethyl]-3-pyridyl]piperazine-1-carboxylate (compound A5)

To a solution of 3-bromo-5-iodo-2-[(1S)-1-methoxyethyl]pyridine (compound A3, 660 mg, 1.9 mmol) and 1-Cbz-piperazine (compound A4, 425.1 mg, 1.9 mmol) in toluene (10 mL) were added cesium carbonate (1.6 g, 4.83 mmol), (R)-BINAP (60.1 mg, 0.1 mmol) and palladium (II) acetate (43.3 mg, 0.19 mmol). The mixture was stirred at 100° C. for 12 hours under N₂ protection. The mixture was filtered and the filtrate was concentrated under vacuum. The residue was purified by silica gel chromatography (EA/PE: 0-50%) to afford benzyl 4-[5-bromo-6-[(1S)-1-methoxyethyl]-3-pyridyl]piperazine-1-carboxylate (compound A5, 740 mg) as a yellow solid. MS calc'd 434.1 (MH⁺), measured 434.1 (MH⁺).

### Step 4: Preparation of 1-[6-[(1S)-1-methoxyethyl]-5-(4,4,5,5-tetramethyl-1,3,2-dioxaborolan-2-yl)-3-pyridyl]-4-methyl-piperazine (Intermediate A)

To a solution of benzyl 4-[5-bromo-6-[(1S)-1-methoxyethyl]-3-pyridyl]piperazine-1-carboxylate (compound A5, 740 mg, 1.7 mmol) and bis(pinacolato)diboron (519.2 mg, 2.04 mmol) in toluene (12 mL) were added KOAc (418.0 mg, 4.26 mmol) and Pd(dppf)Cl₂ (124.7 mg, 0.170 mmol). The reaction mixture was stirred at 90° C. for 12 hrs under N₂ protection. The mixture was filtered and the filtrate was concentrated in vacuo. The residue was purified by silica gel column to afford 1-[6-[(1S)-1-methoxyethyl]-5-(4,4,5,5-tetramethyl-1,3,2-dioxaborolan-2-yl)-3-pyridyl]-4-methyl-piperazine (Intermediate A, 470 mg) as a brown solid. MS calc'd 482.3 (MH⁺), measured 482.2 (MH⁺).

### Intermediate B

### Methyl (3S)-1-[(2S)-3-(4-bromothiazol-2-yl)-2-(tert-butoxycarbonylamino)-propanoyl]hexahydropyridazine-3-carboxylate

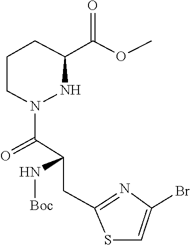 [SMILES: COC(=O)[C@@H]1CCCN(C(=O)[C@H](Cc2nc(Br)cs2)NC(=O)OC(C)(C)C)N1]

The intermediate B was prepared according to the following scheme:

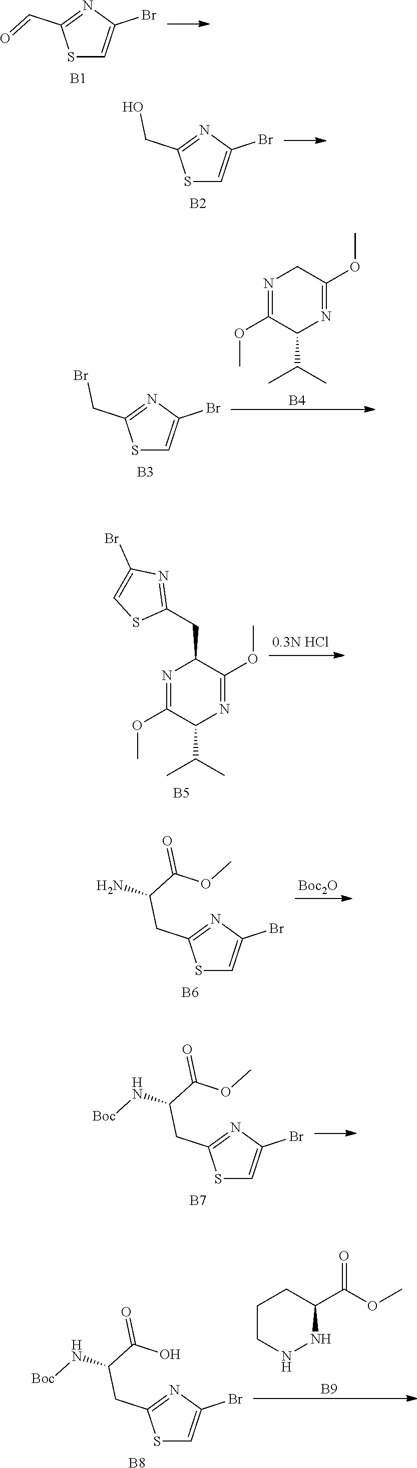

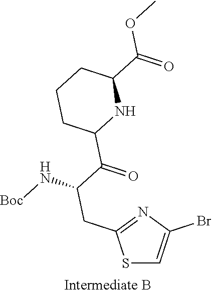

### Step 1: Preparation of (4-bromothiazol-2-yl)methanol (compound B2)

To a solution of 4-bromothiazole-2-carboxaldehyde (compound B1, 6.0 g, 31.25 mmol) in methanol (70 mL) was added sodium borohydride (1.7 g, 46.87 mmol) at 0° C. The mixture was stirred at 25° C. for 1 hour. The reaction was quenched with water (300 mL) at 0° C. and the reaction mixture was extracted by ethyl acetate (200 mL, three times). The combined organic phase was washed with brine (150 mL, twice), dried over anhydrous sodium sulfate, filtered and the filtrate was concentrated under vacuum to afford (4-bromothiazol-2-yl)methanol (compound B2, 6 g) as colorless oil.

### Step 2: Preparation of 4-bromo-2-(bromomethyl)thiazole (compound B3)

To a solution of (4-bromothiazol-2-yl)methanol (compound B2, 6.0 g, 30.92 mmol) in DCM (80 mL) was added CBr₄ (15.4 g, 46.38 mmol) and triphenylphosphine (12.1 g, 46.38 mmol) at 0° C. After being stirred at 25° C. for 1 hour, the mixture was filtered and the filtrate was concentrated under vacuum. The residue was purified by silica gel column, eluted with ethyl acetate in petroleum ether (0˜10%) to afford (4-bromothiazol-2-yl)methanol (compound B3, 6.0 g) as yellow oil. MS calc'd 255.9 (MH⁺), measured 255.9 (MH⁺).

### Step 3: Preparation of 4-bromo-2-[[(2S,5R)-5-isopropyl-3,6-dimethoxy-2,5-dihydropyrazin-2-yl]methyl]thiazole (compound B5)

To a mixture of (R)-2,5-dihydro-3,6-dimethoxy-2-isopropylpyrazine (compound B4, 4.3 g, 23.45 mmol) in THF (60 mL) was added n-butyllithium (10 mL, 25.22 mmol, 2.5 M) at −78° C. slowly. After addition, the mixture was stirred for 0.5 hour at −78° C. 4-bromo-2-(bromomethyl)thiazole (compound B3, 5.4 g, 21.02 mmol) was added into above mixture at −78° C. which was stirred for another 1 hour. The reaction was quenched with saturated solution of NH₄Cl (100 mL) and the reaction mixture was extracted with EtOAc (100 mL, twice). The combined organic layer was washed with brine (150 mL), dried over anhydrous sodium sulfate, filtered and the filtrate was concentrated under vacuum. The residue was purified by reversed-phase chromatography to afford 4-bromo-2-[[(2S,5R)-5-isopropyl-3,6-dimethoxy-2,5-dihydropyrazin-2-yl]methyl]thiazole (compound B5, 3.6 g) as yellow oil. MS calc'd 360 (MH⁺), measured 359.9 (MH⁺).

### Step 4: Preparation of methyl (2S)-2-amino-3-(4-bromothiazol-2-yl)propanoate (compound B6)

To a solution of 4-bromo-2-[[(2S,5R)-5-isopropyl-3,6-dimethoxy-2,5-dihydropyrazin-2-yl]methyl]thiazole (compound B5, 3.6 g, 10 mmol) in ACN (20 mL) was added hydrochloric acid (66.6 mL, 0.3 M). The mixture was stirred at 25° C. for 2 hours. The mixture was basified by saturated solution of NaHCO₃ until pH=8. The mixture was extracted with EtOAc (80 mL, six times). The combined organic layer was dried over anhydrous sodium sulfate, filtered and the filtrate was concentrated under vacuum to afford methyl (2S)-2-amino-3-(4-bromothiazol-2-yl)propanoate (compound B6, 3.1 g) as yellow oil. MS calc'd 264.9 (MH⁺), measured 264.9 (MH⁺).

### Step 5: Preparation of methyl (2S)-3-(4-bromothiazol-2-yl)-2-(tert-butoxycarbonylamino)propanoate (compound B7)

To a solution of methyl (2S)-2-amino-3-(4-bromothiazol-2-yl)propanoate (compound B6, 3.1 g, 11.69 mmol) in DCM (40 mL) were added triethylamine (2.9 g, 29.23 mmol) and (Boc)₂O (3.8 g, 17.54 mmol). After being stirred at 30° C. for 12 hours, the mixture was concentrated under vacuum. The residue was purified by silica gel column, eluted with ethyl acetate in petroleum ether (0˜30%) to afford methyl (2S)-3-(4-bromothiazol-2-yl)-2-(tert-butoxycarbonylamino)propanoate (compound B7, 3.2 g) as yellow oil. MS calc'd 387 (MNa⁺), measured 386.9 (MNa⁺).

### Step 6: Preparation of (2S)-3-(4-bromothiazol-2-yl)-2-(tert-butoxycarbonylamino)-propanoic acid (compound B8)

To a solution of methyl (2S)-3-(4-bromothiazol-2-yl)-2-(tert-butoxycarbonylamino)propanoate (compound B7, 3.2 g, 8.76 mmol) in THF (30 mL), methanol (2 mL) and water (10 mL) was added lithium hydroxide (0.4 mL, 43.81 mmol). After being stirred at 25° C. for 1 hour, the reaction mixture was acidified by 1 M solution of HCl until pH=5. The mixture was extracted with EtOAc (40 mL, twice). The combined organic layer was washed with brine (100 mL), dried over anhydrous sodium sulfate, filtered and the filtrate was concentrated under vacuum to afford (2S)-3-(4-bromothiazol-2-yl)-2-(tert-butoxycarbonylamino)propanoic acid (compound B8, 3.1 g) as yellow oil. MS calc'd 373 (MNa⁺), measured 372.9 (MNa⁺).

### Step 7: Preparation of methyl (3S)-1-[(2S)-3-(4-bromothiazol-2-yl)-2-(tert-butoxycarbonylamino)propanoyl]hexahydropyridazine-3-carboxylate (Intermediate B)

To a solution of (2S)-3-(4-bromothiazol-2-yl)-2-(tert-butoxycarbonylamino)propanoic acid (compound B8, 3.1 g, 8.83 mmol) in DCM (50 mL) was added methyl (3S)-hexahydropyridazine-3-carboxylate;hydrochloride (compound B9, 2.4 g, 13.24 mmol), EDCI (3.4 g, 17.65 mmol), 1-Hydroxybenzotriazole (238.5 mg, 1.77 mmol) and NMM (9.92 mL, 88.26 mmol) at 0° C. After being stirred at 25° C. for 1 hour, the reaction mixture was diluted with water (60 mL) and extracted with EtOAc (60 mL, three times). The combined organic layer was washed with brine (100 mL), dried over anhydrous sodium sulfate, filtered and the filtrate was concentrated under vacuum. The residue was purified by silica gel column and eluted with ethyl acetate in petroleum ether (10˜30%) to afford methyl (3S)-1-[(2S)-3-(4-bromothiazol-2-yl)-2-(tert-butoxycarbonylamino)propanoyl]hexahydropyridazine-3-carboxylate (intermediate B, 2.4 g). MS calc'd 477 (MH⁺), measured 476.9 (MH⁺).

### Intermediate C

### (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

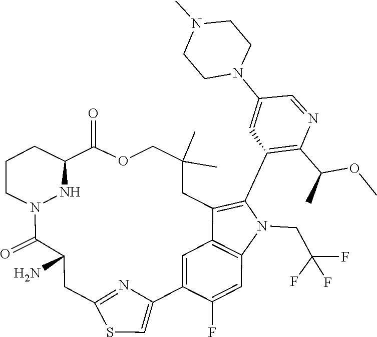

The title intermediate C was prepared according to the following scheme:

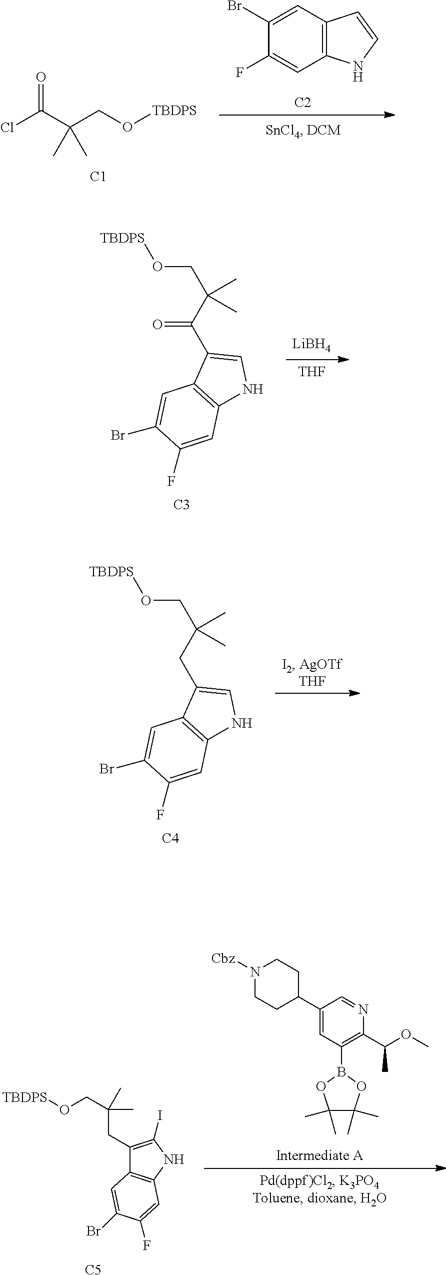

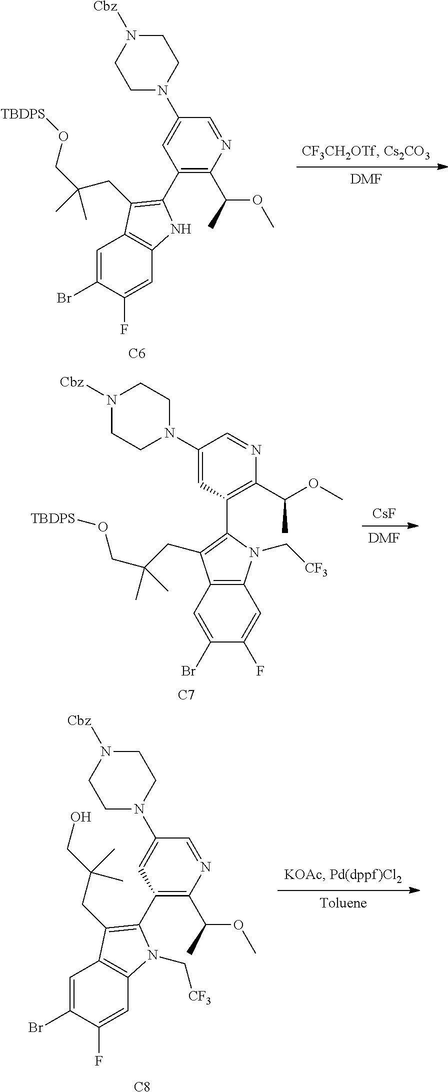

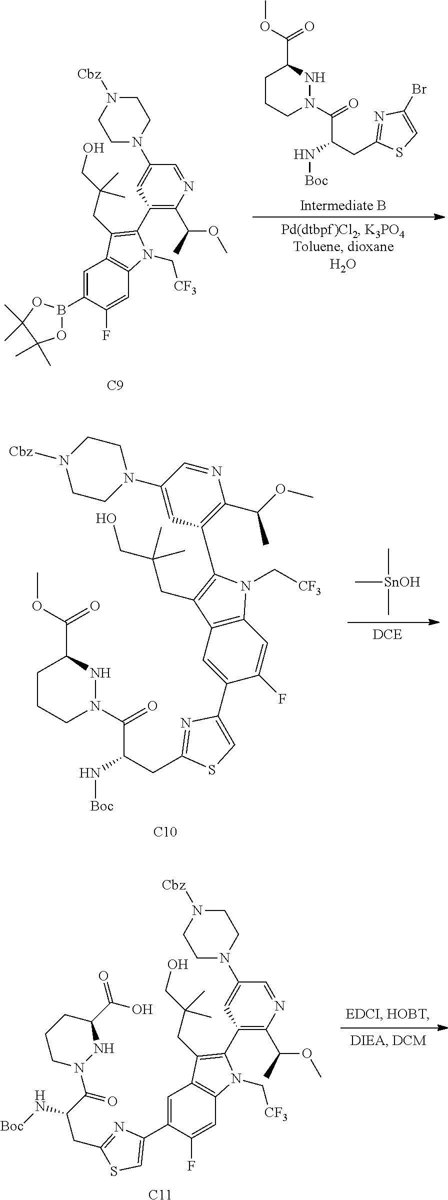

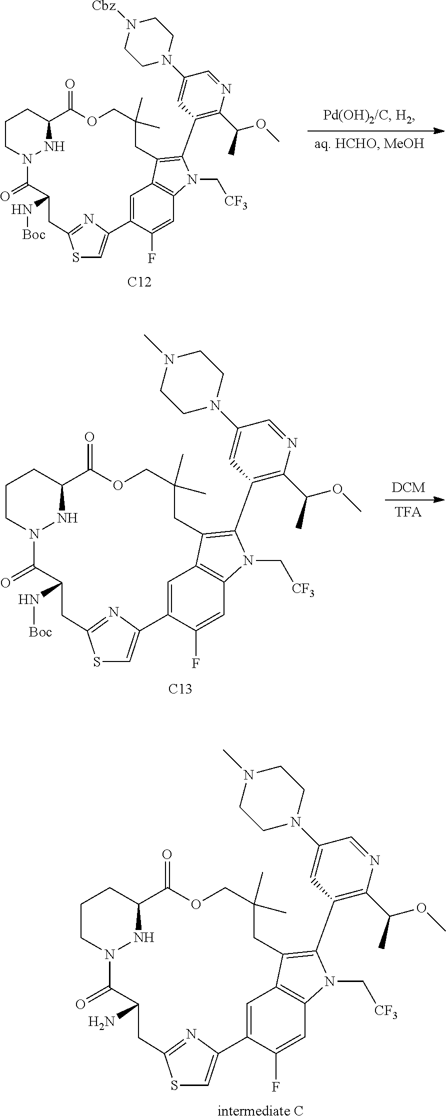

### Step 1: Preparation of 1-(5-bromo-6-fluoro-1H-indol-3-yl)-3-((tert-butyldiphenylsilyl) oxy)-2,2-dimethylpropan-1-one (compound C3)

To a mixture of 3-((tert-butyldiphenylsilyl)oxy)-2,2-dimethylpropanoyl chloride (compound C1, 35.0 g, 116.8 mmol) in DCM (400 mL) at 0° C. was added a solution of SnCl₄ (97.2 mL, 121.5 mmol) slowly. After being stirred at −40° C. for 0.5 hour, 5-bromo-6-fluoro-1H-indole (compound C2, 25.0 g, 116.8 mmol) in DCM (200 mL) was added dropwise to the mixture which was stirred at −40° C. for 15 min. After the reaction was completed, it was quenched with sat·NaHCO₃ aq. (800 mL), and the reaction mixture was extracted with EtOAc (900 mL, twice). The combined organic layer was washed with brine (700 mL), dried over Na₂SO₄, filtered and concentrated in vacuo. The residue was triturated with the solution (100 mL, Petroleum ether:Ethyl acetate=8:1) and filtered. The filter cake was dried in vacuo to afford 1-(5-bromo-6-fluoro-1H-indol-3-yl)-3-((tertbutyldiphenylsilyl)oxy)-2,2-dimethylpropan-1-one (compound C3, 50.0 g) as a yellow solid. MS calc'd 552.1 (MH⁺), measured 552.1 (MH⁺).

### Step 2: Preparation of [3-(5-bromo-6-fluoro-1H-indol-3-yl)-2,2-dimethyl-propoxy]-tert-butyl-diphenyl-silane (compound C4)

To a mixture of 1-(5-bromo-6-fluoro-1H-indol-3-yl)-3-((tertbutyldiphenylsilyl)oxy)-2,2-dimethylpropan-1-one (compound C3, 50.0 g, 90.49 mmol) in THF (600 mL) was added LiBH₄ (48.4 mL, 193.49 mmol, 4 M in THF) dropwise at 0° C. The mixture was stirred at 70° C. for 24 hrs under nitrogen atmosphere. After the reaction was completed, it was quenched by addition of water (600 mL) at 0° C. slowly and the reaction mixture was extracted with EtOAc (600 mL, twice). The combined organic layer was washed with brine (600 mL), dried over Na₂SO₄, filtered and concentrated in vacuo. The residue was purified by silica column chromatography (EtOAc in PE=20%˜33%) to afford [3-(5-bromo-6-fluoro-1H-indol-3-yl)-2,2-dimethyl-propoxy]-tert-butyl-diphenyl-silane (compound C4, 46.0 g) as a white solid. MS calc'd 538.1 (MH⁺), measured 538.2 (MH⁺).

### Step 3: Preparation of [3-(5-bromo-6-fluoro-2-iodo-1H-indol-3-yl)-2,2-dimethyl-propoxy]-tert-butyl-diphenyl-silane (compound C5)

To a mixture of [3-(5-bromo-6-fluoro-1H-indol-3-yl)-2,2-dimethyl-propoxy]-tert-butyl-diphenyl-silane (compound C4, 35.4 g, 65.73 mmol) and iodine (18.4 g, 72.3 mmol) in THF (400 mL) was added silver trifluoromethanesulfonate (20.3 g, 78.88 mmol) at 0° C. The mixture was stirred at 0° C. for 10 min. After the reaction was completed, it was quenched by sat. Na₂SO₃ aq. (400 mL) and EtOAc (400 mL) and the reaction mixture was filtered. The organic layer was washed with brine (100 mL), dried over Na₂SO₄, filtered and concentrated in vacuo. The residue was purified by silica column chromatography (EtOAc in PE=0%˜2.5%) to afford [3-(5-bromo-6-fluoro-2-iodo-1H-indol-3-yl)-2,2-dimethyl-propoxy]-tert-butyl-diphenyl-silane (compound C5, 43.0 g) as a yellow solid. MS calc'd 664.0 (MH⁺), measured 664.1 (MH⁺).

### Step 4: Preparation of benzyl 4-[5-[5-bromo-3-[3-[tert-butyl(diphenyl)silyl]oxy-2,2-dimethyl-propyl]-6-fluoro-1H-indol-2-yl]-6-[(1S)-1-methoxyethyl]-3-pyridyl]piperazine-1-carboxylate (compound C6)

To a mixture of [3-(5-bromo-6-fluoro-2-iodo-1H-indol-3-yl)-2,2-dimethyl-propoxy]-tert-butyl-diphenyl-silane (compound C5, 16.7 g, 25.13 mmol) and benzyl 4-[6-[(1S)-1-methoxyethyl]-5-(4,4,5,5-tetramethyl-1,3,2-dioxaborolan-2-yl)-3-pyridyl]piperazine-1-carboxylate (Intermediate A, 16.7 g, 34.69 mmol) in a mixed solution of 1,4-dioxane (270 mL)/Toluene (90 mL)/water (90 mL) were added potassium phosphate (15.7 g, 73.92 mmol) and Pd(dppf)Cl₂ (920 mg, 1.26 mmol). The mixture was stirred at 70° C. for 12 hrs under nitrogen atmosphere. After the reaction was completed, the mixture was filtered and concentrated in vacuo. The residue was purified by silica column chromatography (EtOAc in PE=20%˜50%) to afford 4-[5-[5-bromo-3-[3-[tert-butyl(diphenyl)silyl]oxy-2,2-dimethyl-propyl]-6-fluoro-1H-indol-2-yl]-6-[(1S)-1-methoxyethyl]-3-pyridyl]piperazine-1-carboxylate (compound C6, 19.5 g) as a white solid. MS calc'd 891.3 (MH⁺), measured 891.3 (MH⁺).

### Step 5: Preparation of benzyl 4-[(5M)-5-[5-bromo-3-[3-[tert-butyl(diphenyl)silyl]oxy-2,2-dimethyl-propyl]-6-fluoro-1-(2,2,2-trifluoroethyl)indol-2-yl]-6-[(1S)-1-methoxyethyl]-3-pyridyl]piperazine-1-carboxylate (compound C7)

To a solution of 4-[5-[5-bromo-3-[3-[tert-butyl(diphenyl)silyl]oxy-2,2-dimethyl-propyl]-6-fluoro-1H-indol-2-yl]-6-[(1S)-1-methoxyethyl]-3-pyridyl]piperazine-1-carboxylate (compound C6, 14.5 g, 16.26 mmol) and Cs₂CO₃ (15.9 g, 48.77 mmol) in DMF (200 mL) was added 2,2,2-trifluoroethyl trifluoromethanesulfonate (37.7 g, 162.56 mmol) dropwise at 0° C., and the mixture was stirred at 20° C. for 12 hrs. After the reaction was completed, EtOAc (70 mL) and water (100 mL) were added and the layers were separated. The aqueous phase was extracted with EtOAc (70 mL, twice). Combined organic layer was washed with brine (100 mL, four times), dried over Na₂SO₄, filtered, and concentrated under vacuum to give a residue. The residue was purified by silica column chromatography to afford benzyl 4-[(5M)-5-[5-bromo-3-[3-[tert-butyl(diphenyl)silyl]oxy-2,2-dimethyl-propyl]-6-fluoro-1-(2,2,2-trifluoroethyl)indol-2-yl]-6-[(1S)-1-methoxyethyl]-3-pyridyl]piperazine-1-carboxylate (compound C7, 8.0 g, PEAK 1, faster eluted) as yellow oil. MS calc'd 973.3 (MH⁺), measured 973.2 (MH⁺).

### Step 6: Preparation of benzyl 4-[(5M)-5-[5-bromo-6-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (compound C8)

To a solution of benzyl 4-[(5M)-5-[5-bromo-3-[3-[tert-butyl(diphenyl)silyl]oxy-2,2-dimethyl-propyl]-6-fluoro-1-(2,2,2-trifluoroethyl)indol-2-yl]-6-[(1S)-1-methoxyethyl]-3-pyridyl]piperazine-1-carboxylate (compound C7, 10.5 g, 10.78 mmol) in DMF (130 mL) was added cesium fluoride (8.2 g, 53.9 mmol) and the mixture was stirred at 60° C. for 24 hrs. After the reaction was completed, EtOAc (100 mL) and water (100 mL) were added and the layers were separated. The aqueous phase was extracted with EtOAc (100 mL, twice). The combined organic layer was washed with brine (80 mL, three times), dried over Na₂SO₄, filtered, and concentrated under vacuum to give a residue. The residue was purified by silica column chromatography (EtOAc in PE=25%˜66%) to afford benzyl 4-[(5M)-5-[5-bromo-6-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (compound C8, 6.5 g) as a yellow solid. MS calc'd 735.2 (MH⁺), measured 735.1 (MH⁺).

### Step 7: Preparation of benzyl 4-[(5M)-5-[6-fluoro-3-(3-hydroxy-2,2-dimethyl-propyl)-5-(4,4,5,5-tetramethyl-1,3,2-dioxaborolan-2-yl)-1-(2,2,2-trifluoroethyl)indol-2-yl]-6-[(1S)-1-methoxyethyl]-3-pyridyl]piperazine-1-carboxylate (compound C9)

To a solution of benzyl 4-[(5M)-5-[5-bromo-6-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (compound C8, 5.4 g), bis(pinacolato)diboron (2.8 g, 11.01 mmol) and potassium acetate (1.2 mL, 18.35 mmol) in toluene (70 mL) was added Pd(dppf)Cl₂ (537.1 mg, 0.73 mmol). The mixture was degassed and purged with nitrogen atmosphere for three times and the mixture was stirred at 90° C. for 12 hrs. After the reaction was completed, the mixture was cooled to room temperature. The reaction mixture was filtered and the filtrate was concentrated in vacuo to give a residue. The residue was purified by silica column chromatography (EtOAc in PE=25%˜66%) to afford benzyl 4-[(5M)-5-[6-fluoro-3-(3-hydroxy-2,2-dimethyl-propyl)-5-(4,4,5,5-tetramethyl-1,3,2-dioxaborolan-2-yl)-1-(2,2,2-trifluoroethyl)indol-2-yl]-6-[(1S)-1-methoxyethyl]-3-pyridyl]piperazine-1-carboxylate (compound C9, 5.2 g) as yellow oil. MS calc'd 783.3 (MH⁺), measured 783.3 (MH⁺).

### Step 8: Preparation of methyl (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)-propanoyl]hexahydropyridazine-3-carboxylate (compound C10)

To a mixture of methyl (3S)-1-[(2S)-3-(4-bromothiazol-2-yl)-2-(tert-butoxycarbonylamino)propanoyl]hexahydropyridazine-3-carboxylate (intermediate B, 2.7 g, 5.69 mmol), benzyl 4-[(5M)-5-[6-fluoro-3-(3-hydroxy-2,2-dimethyl-propyl)-5-(4,4,5,5-tetramethyl-1,3,2-dioxaborolan-2-yl)-1-(2,2,2-trifluoroethyl)indol-2-yl]-6-[(1S)-1-methoxyethyl]-3-pyridyl]piperazine-1-carboxylate (compound C9, 4.9 g, 6.32 mmol) in toluene (60 mL)/1,4-dioxane (20 mL)/water (20 mL) were added K₃PO₄ (3.4 g, 15.81 mmol) and Pd(dtbpf)Cl₂ (412.2 mg, 0.63 mmol) under nitrogen atmosphere. The mixture was stirred at 70° C. for 12 hrs. After the reaction was completed, the mixture was concentrated in vacuo to give a residue. The residue was purified by silica column (EtOAc in PE=10%˜75%) to afford methyl (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)-propanoyl]hexahydropyridazine-3-carboxylate (compound C10, 3.6 g) as a brown solid. MS calc'd 1053.4 (MH⁺), measured 1053.3 (MH⁺).

### Step 9: Preparation of (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)propanoyl]hexahy-dropyridazine-3-carboxylic acid (compound C11)

To a solution of methyl (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)-propanoyl]-hexahydropyridazine-3-carboxylate (compound C10, 3.6 g, 3.42 mmol) in DCE (50 mL) was added trimethylstannanol (2.4 g, 13.67 mmol) and the mixture was stirred at 60° C. for 12 hrs. After the reaction was completed, EtOAc (80 mL) and water (60 mL) were added and the layers were separated. The aqueous phase was extracted with EtOAc (80 mL, twice). The combined organic layer was washed with brine (100 mL), dried over Na₂SO₄, filtered, and concentrated under vacuum to give (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)propanoyl]hexahy-dropyridazine-3-carboxylic acid (compound C11, 4.3 g) as a brown solid. MS calc'd 1039.4 (MH⁺), measured 1039.2 (MH⁺).

### Step 10: Preparation of benzyl 4-[5-[(7S,13S)-7-(tert-butoxycarbonylamino)-24-fluoro-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-(20M)-20-yl]-6-[(1S)-1-methoxyethyl]-3-pyridyl]piperazine-1-carboxylate (compound C12)

To a mixture of (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)propanoyl]hexahy-dropyridazine-3-carboxylic acid (compound C11, 4.3 g, 4.14 mmol) in DCM (430 mL) was added DIEA (14.4 mL, 82.76 mmol), EDCI (11.9 g, 62.07 mmol) and 1-hydroxybenzotriazole (1.4 g, 10.35 mmol) at 0° C. The mixture was stirred at 15° C. for 12 hrs. After the reaction was completed, the mixture was concentrated in vacuo, then diluted with water (80 mL), extracted with EtOAc (80 mL, twice). The combined organic layer was washed with brine (80 mL), dried over Na₂SO₄, filtered and concentrated in vacuo. The residue was purified by silica column chromatography (EtOAc in PE=25%˜66%) to afford benzyl 4-[5-[(7S,13S)-7-(tert-butoxycarbonylamino)-24-fluoro-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-(20M)-20-yl]-6-[(1S)-1-methoxyethyl]-3-pyridyl]piperazine-1-carboxylate (compound C12, 3.1 g) as yellow gum. MS calc'd 1021.4 (MH⁺), measured 1021.2 (MH⁺).

### Step 11: Preparation of tert-butyl N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound C13)

To a mixture of benzyl 4-[5-[(7S,13S)-7-(tert-butoxycarbonylamino)-24-fluoro-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-(20M)-20-yl]-6-[(1S)-1-methoxyethyl]-3-pyridyl]piperazine-1-carboxylate (compound C12, 3.1 g, 3.04 mmol) and formaldehyde aqueous (775.0 mg, 9.55 mmol) in methanol (150 mL) was added Pd(OH)₂ on activated carbon (2.79 g, 3.97 mmol). The mixture was degassed and purged with H₂ three times. The mixture was hydrogenated at 30° C. for 18 hrs. After the reaction was completed, the mixture was filtered and the filtrate was concentrated in vacuo to afford tert-butyl N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound C13, 2.6 g) as a brown solid. MS calc'd 901.3 (MH⁺), measured 901.3 (MH⁺).

### Step 12: Preparation of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate C)

To a mixture of tert-butyl N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound C13, 2.6 g, 2.89 mmol) in DCM (18 mL) was added TFA (14.0 mL, 181.72 mmol). The mixture was stirred at 15° C. for 0.5 h. After the reaction was completed, the mixture was concentrated in vacuo and diluted with sat. NaHCO₃ (30 mL), extracted with EtOAc (30 mL, three times). The combined organic layer was washed with brine (50 mL), dried over Na₂SO₄, filtered and concentrated in vacuo to afford (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate C, 2.0 g) as a yellow solid, which was used directly in the next step. MS calc'd 801.3 (MH⁺), measured 801.2 (MH⁺)

### Intermediate D

### (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

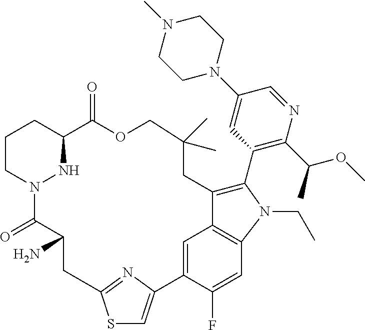

The title compound was prepared in analogy to the preparation of Intermediate C by using iodoethane instead of 2,2,2-trifluoroethyl trifluoromethanesulfonate.

### Intermediate E

### (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

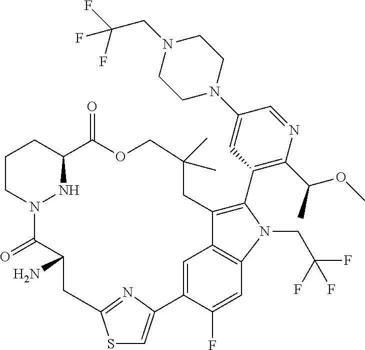

The compound was prepared according to the following scheme:

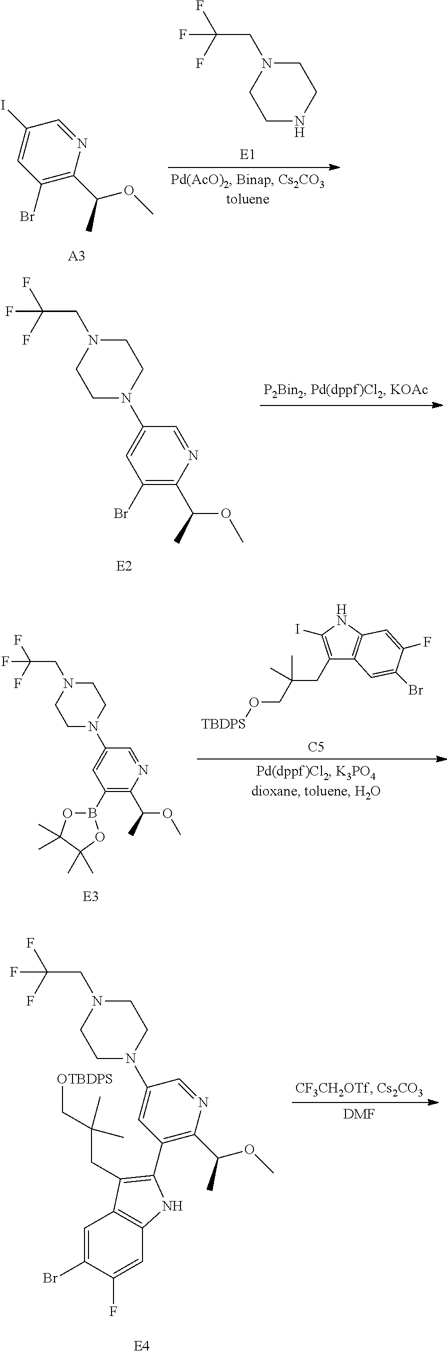

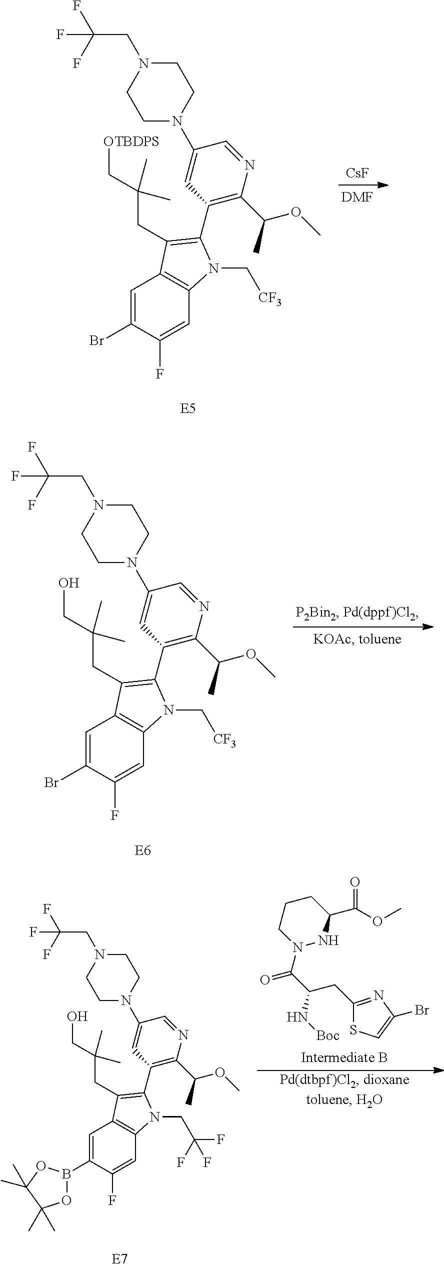

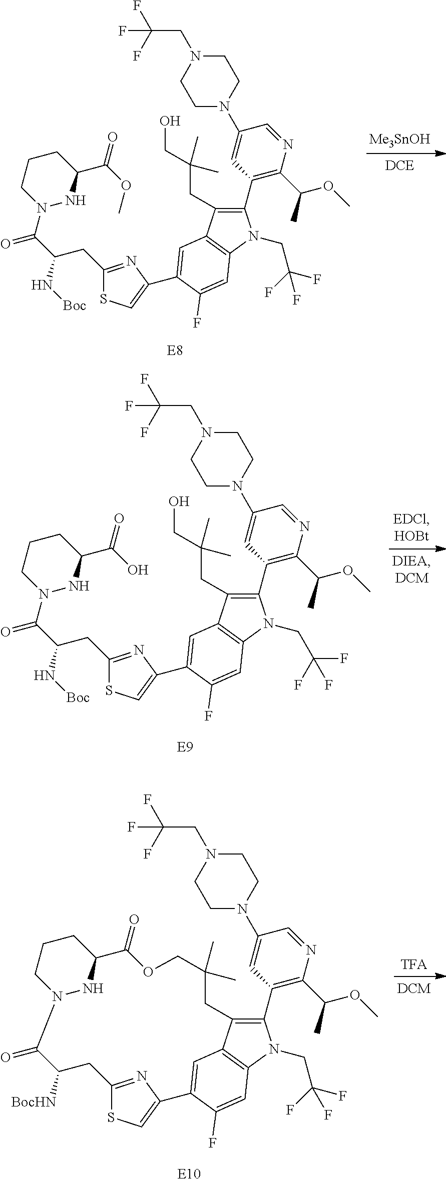

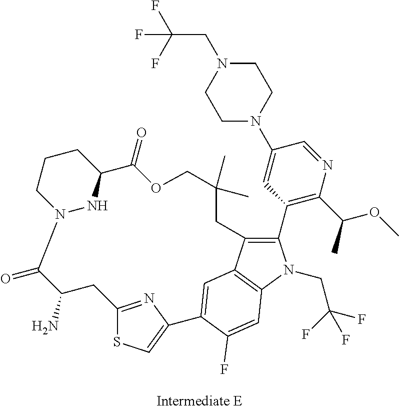

### Step 1: Preparation of 1-[5-bromo-6-[(1S)-1-methoxyethyl]-3-pyridyl]-4-(2,2,2-trifluoroethyl)piperazine (compound E2)

To a mixture of 3-bromo-5-iodo-2-[(1S)-1-methoxyethyl]pyridine (compound A3, 2.03 g, 5.95 mmol) and 1-(2,2,2-trifluoroethyl)piperazine (compound E1, 1.0 g, 5.95 mmol) in toluene (15 mL) were added Cs₂CO₃ (4.85 g, 14.88 mmol), (R)-binap (92.6 mg, 0.15 mmol) and Pd(OAc)₂ (66.8 mg, 0.3 mmol). The reaction mixture was degassed and purged with nitrogen for 3 times and the mixture was stirred at 100° C. for 12 hrs under nitrogen atmosphere. After being cooled to room temperature, the reaction mixture was filtered and the filtrate was concentrated in vacuo to give a residue. The residue was purified by column chromatography to 1-[5-bromo-6-[(1S)-1-methoxyethyl]-3-pyridyl]-4-(2,2,2-trifluoroethyl)piperazine (compound E2, 2.0 g) as yellow oil. MS calc'd 382.2 (MH⁺), measured 382.1 (MH⁺)

### Step 2: 1-[6-[(1S)-1-methoxyethyl]-5-(4,4,5,5-tetramethyl-1,3,2-dioxaborolan-2-yl)-3-pyridyl]-4-(2,2,2-trifluoroethyl)piperazine (compound E3)

To a solution of 1-[5-bromo-6-[(1S)-1-methoxyethyl]-3-pyridyl]-4-(2,2,2-trifluoroethyl)piperazine (compound E2, 3.2 g, 8.37 mmol), bis(pinacolato)diboron (3.19 g, 12.56 mmol) and KOAc (2.1 g, 20.93 mmol) in toluene (50 mL) was added Pd(dppf)Cl₂ (306.3 mg, 0.42 mmol). The mixture was degassed and purged with nitrogen for 3 times and the mixture was stirred at 90° C. for 12 hrs under nitrogen atmosphere. After being cooled to the room temperature, the reaction mixture was filtered, the filtrate was concentrated in vacuo to give a residue, which was purified by reversed phase column to afford 1-[6-[(1S)-1-methoxyethyl]-5-(4,4,5,5-tetramethyl-1,3,2-dioxaborolan-2-yl)-3-pyridyl]-4-(2,2,2-trifluoroethyl)piperazine (compound E3, 1.9 g) as a yellow gum. MS calc'd 430.2 (MH⁺), measured 348.4 (M-C₆H₁₀+H⁺).

### Step 3: Preparation of [3-[5-bromo-6-fluoro-2-[2-[(1S)-1-methoxyethyl]-5-[4-(2,2,2-trifluoroethyl)piperazin-1-yl]-3-pyridyl]-1H-indol-3-yl]-2,2-dimethyl-propoxy]-tert-butyl-diphenyl-silane (compound E4)

To a solution of 1-[6-[(1S)-1-methoxyethyl]-5-(4,4,5,5-tetramethyl-1,3,2-dioxaborolan-2-yl)-3-pyridyl]-4-(2,2,2-trifluoroethyl)piperazine (compound E3, 1.9 g, 4.41 mmol), [3-(5-bromo-6-fluoro-2-iodo-1H-indol-3-yl)-2,2-dimethyl-propoxy]-tert-butyl-diphenyl-silane (compound C5, 2.1 g, 3.15 mmol) in 1,4-dioxane (24 mL), water (8 mL) and toluene (8 mL) was added K₃PO₄ (2.1 g, 9.5 mmol) and Pd(dppf)Cl₂ (231 mg, 0.37 mmol). The mixture was degassed by bubbling nitrogen for 2 min, and the reaction mixture was stirred at 70° C. for 12 hrs. After being cooled to room temperature, the reaction mixture was filtered. The filtrate was concentrated in vacuo to give a residue. The residue was purified by column chromatography (EtOAc in PE: 30%-60%) to afford [3-[5-bromo-6-fluoro-2-[2-[(1S)-1-methoxyethyl]-5-[4-(2,2,2-trifluoroethyl)piperazin-1-yl]-3-pyridyl]-1H-indol-3-yl]-2,2-dimethyl-propoxy]-tert-butyl-diphenyl-silane (compound E4, 960.0 mg) as a yellow gum. MS calc'd 839.3 (MH⁺), measured 839.3 (MH⁺)

### Step 4: Preparation of [3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propoxy]-tert-butyl-diphenyl-silane (compound E5)

To a solution of [3-[5-bromo-6-fluoro-(2M)-2-[2-[(1S)-1-methoxyethyl]-5-[4-(2,2,2-trifluoroethyl)piperazin-1-yl]-3-pyridyl]-1H-indol-3-yl]-2,2-dimethyl-propoxy]-tert-butyl-diphenyl-silane (compound E4, 1 g, 1.14 mmol) in DMF (35 mL) was added Cs₂CO₃ (1.1 g, 3.44 mmol) and 2,2,2-trifluoroethyl trifluoromethanesulfonate (2.7 g, 11.63 mmol) at 0° C. After being stirred at 20° C. for 15 hrs, the reaction mixture was poured into water (100 mL), and extracted with EtOAc (50 mL, three times). The combined organic was washed with brine (50 mL, three times), dried over Na₂SO₄, filtered and concentrated under vacuum to give a residue which was purified by column chromatography (EtOAc in PE: 30%-40%) to afford [3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propoxy]-tert-butyl-diphenyl-silane (compound E5, 640.0 mg, faster eluted) as a white solid. MS calc'd 921.3 (MH⁺), measured 921.4 (MH⁺).

### Step 5: Preparation of 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E6)

To a solution of [3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propoxy]-tert-butyl-diphenyl-silane (compound E5, 640.0 mg, 0.69 mmol) in DMF (7 mL) was added cesium fluoride (421.8 mg, 2.78 mmol). The mixture was stirred at 60° C. for 16 hrs. After being cooled to room temperature, the reaction mixture was filtered and the filtrate was concentrated in vacuo to give a residue. The residue was purified by column chromatography (EtOAc in PE: 30%-60%) to afford 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E6, 360.0 mg) as yellow oil. MS calc'd 683.2 (MH⁺), measured 683.1 (MH⁺).

### Step 6: Preparation of 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E7)

To a solution of 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E6, 360.0 mg, 0.53 mmol), bis(pinacolato)diboron (200.6 mg, 0.79 mmol) in toluene (6 mL) was added potassium acetate (0.08 mL, 1.32 mmol) and Pd(dppf)Cl₂ (40 mg, 0.1 mmol). The reaction mixture was degassed by bubbling nitrogen for 5 min then stirred at 80° C. for 15 hrs. After being cooled to room temperature, the reaction mixture was filtered and the filtrate was concentrated in vacuo to give a residue. The residue was purified by column chromatography (EtOAc in PE: 30%-50%) to afford 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E7, 300.0 mg) as yellow gum. MS calc'd 731.4 (MH⁺), measured 731.4 (MH⁺).

### Step 7: Preparation of methyl (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylate (compound E8)

To a mixture of 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E7, 0.3 g, 0.41 mmol) and methyl (3S)-1-[(2S)-3-(4-bromothiazol-2-yl)-2-(tert-butoxycarbonylamino)propanoyl]hexahydropyridazine-3-carboxylate (intermediate B, 196.7 mg, 0.41 mmol) in toluene (3 mL), 1,4-dioxane (1 mL) and water (1 mL) were added K₃PO₄ (221.3 mg, 1.04 mmol) and Pd(dtbpf)Cl₂ (27.05 mg, 0.04 mmol). The mixture was stirred at 70° C. for 12 hrs under nitrogen atmosphere. After being cooled to room temperature, the reaction mixture was filtered and the filtrate was concentrated in vacuo to give a residue. The residue was purified by column chromatography (EtOAc in PE: 60%-80%) to afford methyl (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylate (compound E8, 200.0 mg) as yellow gum. MS calc'd 1001.4 (MH⁺), measured 1001.4 (MH⁺).

### Step 8: Preparation of (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylic acid (compound E9)

To a mixture of methyl (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylate (compound E8, 200.0 mg, 0.2 mmol) in DCE (5 mL) was added Me₃SnOH (200.0 mg, 1.11 mmol). The mixture was stirred at 60° C. for 12 hrs. The reaction mixture was concentrated under vacuum to give a residue. EtOAc (10 mL) and water (10 mL) were added to the residue and the layers were separated. The aqueous phase was extracted with EtOAc (15 mL, twice). The combined organic layer was washed with brine (20 mL), dried over Na₂SO₄, filtered, and concentrated under vacuum to afford (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylic acid (compound E9, 188.0 mg) as a brown solid. MS calc'd 987.4 (MH⁺), measured 987.4 (MH⁺).

### Step 9: Preparation of tert-butyl N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound E10)

To a mixture of (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylic acid (compound E9, 188.0 mg, 0.19 mmol) in DCM (20 mL) were added DIEA (0.7 mL, 3.81 mmol), EDCI (550.0 mg, 2.87 mmol) and HOBt (65.0 mg, 0.48 mmol) at 0° C. After being stirred at 20° C. for 12 hrs, the reaction mixture was poured into water (20 mL) and extracted with EtOAc (20 mL, three times). The combined organic layer was washed with brine (30 mL), dried over Na₂SO₄, filtered and concentrated under vacuum to give a residue which was purified by column chromatography (EtOAc in PE: 50%-70%) to afford tert-butyl N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound E10, 110.0 mg) as a yellow solid. MS calc'd 969.4 (MH⁺), measured 969.5 (MH⁺).

### Step 10: Preparation of (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E)

To a solution of tert-butyl N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound E10, 110.0 mg, 0.11 mmol) in DCM (1 mL) was added TFA (1.0 mL, 12.98 mmol). The mixture was stirred at 20° C. for 1 h. After the reaction was completed, the reaction mixture was concentrated under vacuum to give a residue. Sat. NaHCO₃ aq. (20 mL) was added and the mixture was extracted with EtOAc (15 mL, three times). The combined organic layer was washed with brine (20 mL), dried over anhydrous sodium sulfate, filtered and concentrated in vacuo to afford (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E, 98.0 mg) as a yellow solid. MS calc'd 869.4 (MH⁺), measured 869.2 (MH⁺).

### Intermediate F

### (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

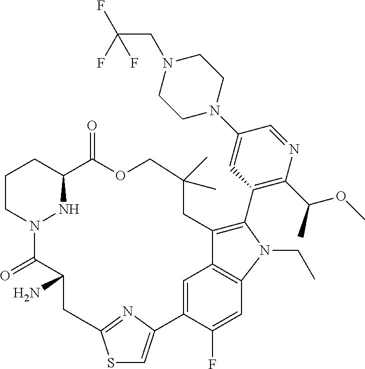

The title compound was prepared in analogy to the preparation of Intermediate E by using iodoethane instead of 2,2,2-trifluoroethyl trifluoromethanesulfonate.

### Intermediate G

### (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione

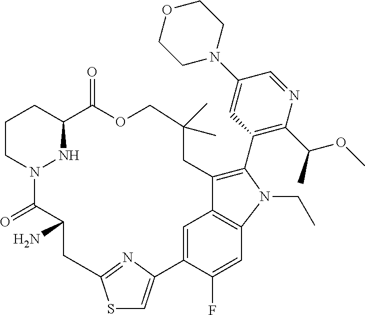

The compound was prepared according to the following scheme:

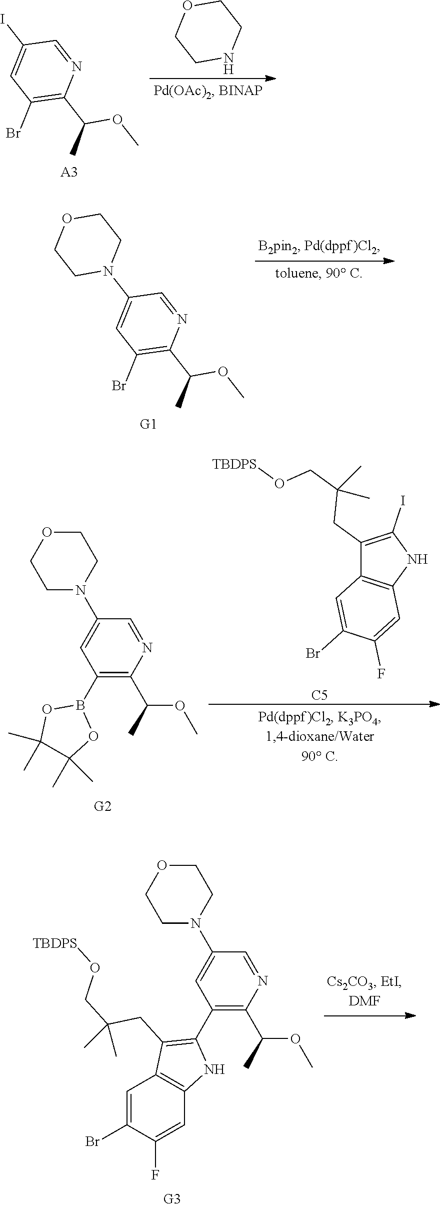

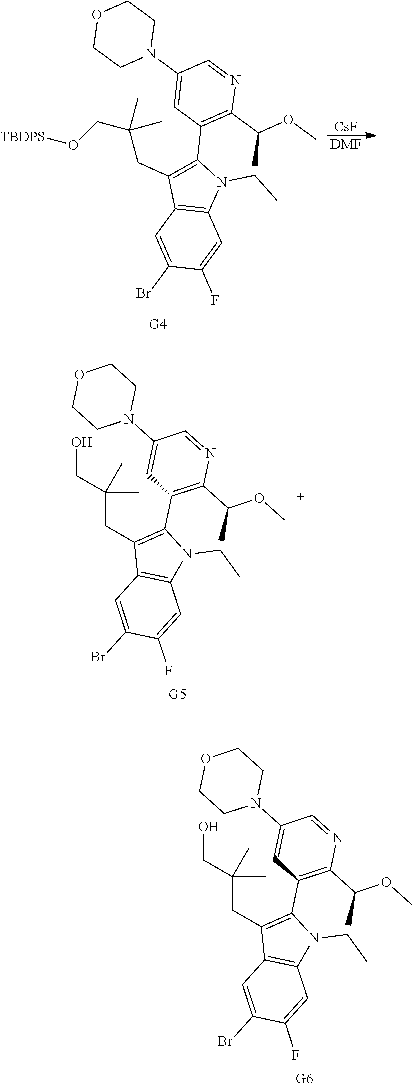

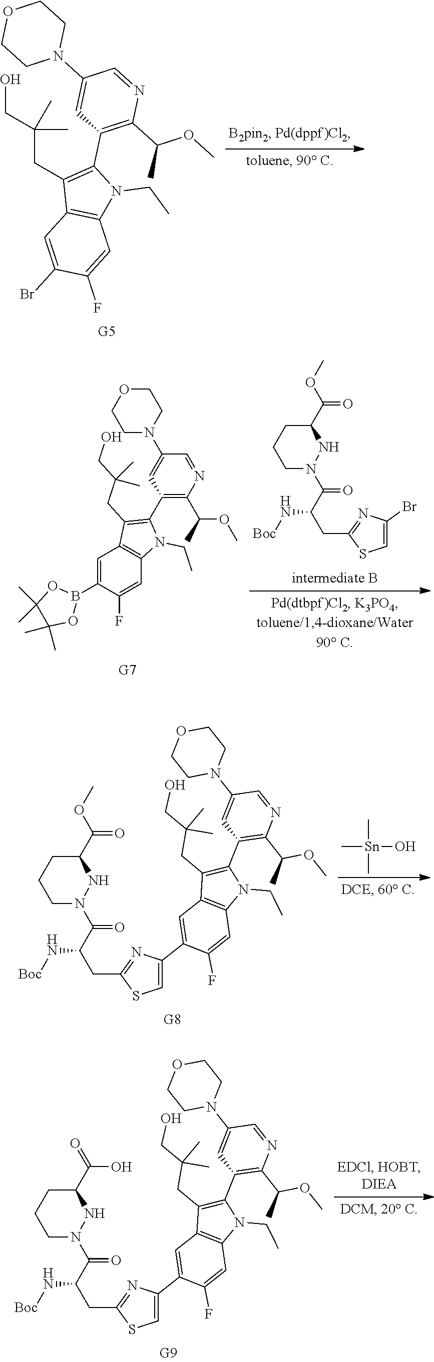

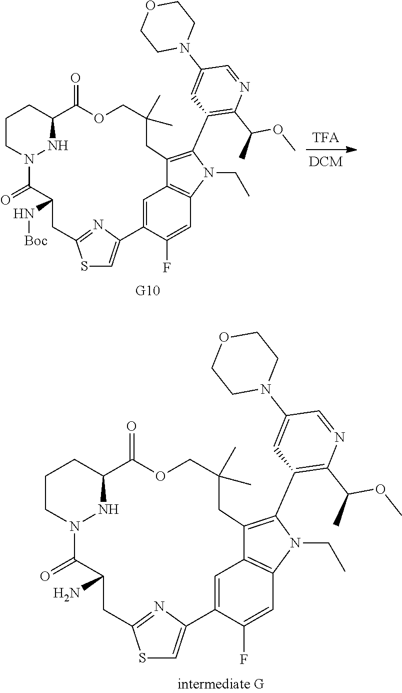

### Step 1: Preparation of 4-[5-bromo-6-[(1S)-1-methoxyethyl]-3-pyridyl]morpholine (compound G1)

To a mixture of 3-bromo-5-iodo-2-[(1S)-1-methoxyethyl]pyridine (compound A3, 30 g, 87.73 mmol) and morpholine (7.6 g, 87.73 mmol) in toluene (450 mL) were added Cs₂CO₃ (57.2 g, 175.45 mmol), (R)-binap (2.7 g, 4.39 mmol) and Pd(OAc)₂ (0.98 g, 4.39 mmol). The reaction mixture was degassed and purged with nitrogen for 3 times and the mixture was stirred at 90° C. for 12 hrs under nitrogen atmosphere. After being cooled to room temperature, the reaction mixture was filtered and the filtrate was concentrated in vacuo to give a residue. The residue was purifed by column chromatography to afford 4-[5-bromo-6-[(1S)-1-methoxyethyl]-3-pyridyl]morpholine (compound G1, 21 g) as yellow oil. MS calc'd 301.1 (MH⁺), measured 301.1 (MH⁺).

### Step 2: Preparation of 4-[6-[(1S)-1-methoxyethyl]-5-(4,4,5,5-tetramethyl-1,3,2-dioxaborolan-2-yl)-3-pyridyl]morpholine (compound G2)

To a solution of 4-[5-bromo-6-[(1S)-1-methoxyethyl]-3-pyridyl]morpholine (compound G1, 21 g, 63.3 mmol), bis(pinacolato)diboron (24.0 g, 94.63 mmol) and KOAc (13.6 g, 138.79 mmol) in toluene (500 mL) was added Pd(dppf)Cl₂ (4.4 g, 6.31 mmol). The mixture was degassed and purged with nitrogen for 3 times and the mixture was stirred at 90° C. for 12 hrs under nitrogen atmosphere. After being cooled to the room temperature, the reaction mixture was filtered, the filtrate was concentrated in vacuo to give crude product 4-[6-[(1S)-1-methoxyethyl]-5-(4,4,5,5-tetramethyl-1,3,2-dioxaborolan-2-yl)-3-pyridyl]morpholine (compound G2, 45 g) as a yellow gum, which was used to the next step. MS calc'd 349.2 (MH⁺), measured 349.2 (MH⁺).

### Step 3: Preparation of [3-[5-bromo-6-fluoro-2-[2-[(1S)-1-methoxyethyl]-5-morpholino-3-pyridyl]-1H-indol-3-yl]-2,2-dimethyl-propoxy]-tert-butyl-diphenyl-silane (compound G3)

To a solution of 4-[6-[(1S)-1-methoxyethyl]-5-(4,4,5,5-tetramethyl-1,3,2-dioxaborolan-2-yl)-3-pyridyl]morpholine (compound G2, 40.6 g, 46.65 mmol), [3-(5-bromo-6-fluoro-2-iodo-1H-indol-3-yl)-2,2-dimethyl-propoxy]-tert-butyl-diphenyl-silane (compound C5, 31 g, 46.65 mmol) in 1,4-dioxane (420 mL) and water (80 mL) was added K₃PO₄ (29.7 g, 2.33 mmol) and Pd(dppf)Cl₂ (1.7 g, 0.29 mmol). The mixture was degassed by bubbling nitrogen for 2 min, and the reaction mixture was stirred at 90° C. for 18 hrs. After being cooled to room temperature, the reaction mixture was extracted with EA (200 mL, three times). The combined organic layer was washed with brine (200 mL), dried over Na₂SO₄, filtered and the filtrate was concentrated in vacuo to give a residue. The residue was purified by column chromatography to afford [3-[5-bromo-6-fluoro-2-[2-[(1S)-1-methoxyethyl]-5-morpholino-3-pyridyl]-1H-indol-3-yl]-2,2-dimethyl-propoxy]-tert-butyl-diphenyl-silane (compound G3, 17.2 g) as yellow oil. MS calc'd 758.3 (MH⁺), measured 758.3 (MH⁺).

### Step 4: Preparation of [3-[5-bromo-1-ethyl-6-fluoro-2-[2-[(1S)-1-methoxyethyl]-5-morpholino-3-pyridyl]indol-3-yl]-2,2-dimethyl-propoxy]-tert-butyl-diphenyl-silane (compound G4)

To a solution of [3-[5-bromo-6-fluoro-2-[2-[(1S)-1-methoxyethyl]-5-morpholino-3-pyridyl]-1H-indol-3-yl]-2,2-dimethyl-propoxy]-tert-butyl-diphenyl-silane (compound G3, 15 g, 19.77 mmol) in DMF (300 mL) was added Cs₂CO₃ (19.3 g, 59.3 mmol) and iodoethane (6.16 g, 39.53 mmol) at 0° C. After being stirred at 20° C. for 16 hrs, the reaction mixture was poured into water (200 mL), and extracted with EtOAc (200 mL, three times). The combined organic layer was washed with brine (10 mL, three times), dried over Na₂SO₄, filtered and concentrated under vacuum to give a residue. The residue was purified by column chromatography to afford [3-[5-bromo-1-ethyl-6-fluoro-2-[2-[(1S)-1-methoxyethyl]-5-morpholino-3-pyridyl]indol-3-yl]-2,2-dimethyl-propoxy]-tert-butyl-diphenyl-silane (compound G4, 14.7 g) as yellow oil. MS calc'd 786.3 (MH⁺), measured 786.4 (MH⁺).

### Step 5: Preparation of 3-[5-bromo-1-ethyl-6-fluoro-(2M)-2-[2-[(1S)-1-methoxyethyl]-5-morpholino-3-pyridyl]indol-3-yl]-2,2-dimethyl-propan-1-ol (compound G5) and 3-[5-bromo-1-ethyl-6-fluoro-(2P)-2-[2-[(1S)-1-methoxyethyl]-5-morpholino-3-pyridyl]indol-3-yl]-2,2-dimethyl-propan-1-ol (compound G6)

To a solution of [3-[5-bromo-1-ethyl-6-fluoro-2-[2-[(1S)-1-methoxyethyl]-5-morpholino-3-pyridyl]indol-3-yl]-2,2-dimethyl-propoxy]-tert-butyl-diphenyl-silane (compound G4, 14.7 g, 18.68 mmol) in DMF (160 mL) was added cesium fluoride (14.2 g, 93.41 mmol). The mixture was stirred at 60° C. for 48 hrs. After being cooled to room temperature, the reaction mixture were added with EtOAc (300 mL) and water (300 mL) and the layers were separated. The aqueous phase was extracted with EtOAc (200 mL, three times). The combined organic layer was washed with brine (200 mL, four times), dried over Na₂SO₄, filtered, and concentrated under vacuum to give a residue. The residue was purified by column chromatography to afford 3-[5-bromo-1-ethyl-6-fluoro-(2M)-2-[2-[(1S)-1-methoxyethyl]-5-morpholino-3-pyridyl]indol-3-yl]-2,2-dimethyl-propan-1-ol (compound G5, 6 g, faster eluted) as colorless foam and 3-[5-bromo-1-ethyl-6-fluoro-(2P)-2-[2-[(1S)-1-methoxyethyl]-5-morpholino-3-pyridyl]indol-3-yl]-2,2-dimethyl-propan-1-ol (compound G6, 4.5 g, slower eluted) as colorless foam. Compound G5: MS calc'd 548.2 (MH⁺), measured 548.2 (MH⁺). ¹H NMR (400 MHz, Methanol-d₄) δ=8.41 (d, J=2.4 Hz, 1H), 7.92 (d, J=6.8 Hz, 1H), 7.37-7.33 (m, 2H), 4.58 (s, 1H), 4.05-3.98 (m, 2H), 3.87-3.82 (m, 5H), 3.27-3.23 (m, 4H), 3.15-3.13 (m, 1H), 3.00 (s, 3H), 2.75-2.71 (m, 1H), 2.24-2.22 (m, 1H), 1.42 (d, J=6.4 Hz, 3H), 1.22 (t, J=7.2 Hz, 3H), 0.76 (s, 3H), 0.76 (s, 3H).

### X-Ray Crystallographic Analysis of Compound G5

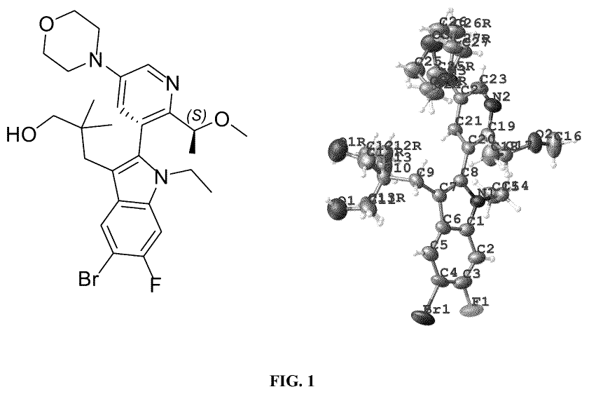

Absolute configuration structure of compound G5 was confirmed by X-ray crystallographic analysis of its single crystal. (FIG. 1).

### Step 6: Preparation of 3-[1-ethyl-6-fluoro-(2M)-2-[2-[(1S)-1-methoxyethyl]-5-morpholino-3-pyridyl]-5-(4,4,5,5-tetramethyl-1,3,2-dioxaborolan-2-yl)indol-3-yl]-2,2-dimethyl-propan-1-ol (compound G7)

To a solution of 3-[5-bromo-1-ethyl-6-fluoro-(2M)-2-[2-[(1S)-1-methoxyethyl]-5-morpholino-3-pyridyl]indol-3-yl]-2,2-dimethyl-propan-1-ol (compound G5, 6 g, 10.94 mmol), bis(pinacolato)diboron (4.2 g, 16.41 mmol) in toluene (60 mL) was added potassium acetate (2.7 g, 27.35 mmol) and Pd(dppf)Cl₂ (0.8 g, 1.09 mmol). The reaction mixture was degassed by bubbling nitrogen for 5 min then stirred at 90° C. for 15 hrs. After being cooled to room temperature, the reaction mixture was filtered and the filtrate was concentrated in vacuo to give a residue. The residue was purified by column chromatography to afford 3-[1-ethyl-6-fluoro-(2M)-2-[2-[(1S)-1-methoxyethyl]-5-morpholino-3-pyridyl]-5-(4,4,5,5-tetramethyl-1,3,2-dioxaborolan-2-yl)indol-3-yl]-2,2-dimethyl-propan-1-ol (compound G7, 4.5 g) as colorless gum. MS calc'd 596.4 (MH⁺), measured 596.4 (MH⁺).

### Step 7: Preparation of methyl (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylate (compound G8)

To a mixture of 3-[1-ethyl-6-fluoro-(2M)-2-[2-[(1S)-1-methoxyethyl]-5-morpholino-3-pyridyl]-5-(4,4,5,5-tetramethyl-1,3,2-dioxaborolan-2-yl)indol-3-yl]-2,2-dimethyl-propan-1-ol (compound G7, 4.5 g, 7.56 mmol) and methyl (3S)-1-[(2S)-3-(4-bromothiazol-2-yl)-2-(tert-butoxycarbonylamino)propanoyl]hexahydropyridazine-3-carboxylate (intermediate B, 3.6 g, 7.56 mmol) in toluene (45 mL), 1,4-dioxane (15 mL) and water (15 mL) were added K₃PO₄ (4.0 g, 18.89 mmol) and Pd(dtbpf)Cl₂ (492.5 mg, 0.75 mmol). The mixture was stirred at 70° C. for 12 hrs under nitrogen atmosphere. After being cooled to room temperature, the reaction mixture was filtered and the filtrate was concentrated in vacuo to give a residue. The residue was purified by column chromatography to afford methyl (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylate (compound G8, 3.8 g) as colorless gum. MS calc'd 866.4 (MH⁺), measured 866.4 (MH⁺).

### Step 8: Preparation of (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylic acid (compound G9)

To a mixture of methyl (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylate (compound G8, 3.8 g, 4.39 mmol) in DCE (76 mL) was added Me₃SnOH (3.2 g, 17.55 mmol). The mixture was stirred at 60° C. for 48 hrs. The reaction mixture was concentrated under vacuum to give a residue. EtOAc (200 mL) and water (100 mL) were added to the residue and the layers were separated. The aqueous phase was extracted with EtOAc (150 mL, twice). The combined organic layer was washed with brine (200 mL), dried over Na₂SO₄, filtered, and concentrated under vacuum to afford (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylic acid (compound G9, 3.7 g) as a brown solid. MS calc'd 852.4 (MH⁺), measured 852.4 (MH⁺).

### Step 9: Preparation of tert-butyl N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound G10)

To a mixture of (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylic acid (compound G9, 2.5 g, 2.93 mmol) in DCM (250 mL) were added DIEA (7.58 mL, 58.68 mmol), EDCI (8.4 g, 44.01 mmol) and HOBt (991.2 mg, 0.91 mmol) at 0° C. After being stirred at 20° C. for 12 hrs, the reaction mixture was poured into water (100 mL) and extracted with EtOAc (100 mL, three times). The combined organic layer was washed with brine (30 mL), dried over Na₂SO₄, filtered and concentrated under vacuum to give a residue which was purified by column chromatography to afford tert-butyl N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound G10, 1.2 g) as yellow oil. MS calc'd 834.4 (MH⁺), measured 834.4 (MH⁺).

### Step 10: Preparation of (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate G)

To a solution of tert-butyl N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound G10, 1.2 g, 1.44 mmol) in DCM (12 mL) was added TFA (6.0 mL). The mixture was stirred at 20° C. for 3 hrs. After the reaction was completed, the reaction mixture was concentrated under vacuum to give a residue. Sat. NaHCO₃ aq. (60 mL) was added and the mixture was extracted with EtOAc (80 mL, three times). The combined organic layer was washed with brine (100 mL), dried over anhydrous sodium sulfate, filtered and concentrated in vacuo to afford (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate G, 1 g) as a yellow solid. MS calc'd 734.3 (MH⁺), measured 734.3 (MH⁺).

### Intermediate H

### (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

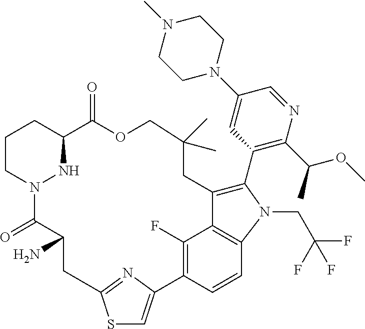

The title compound was prepared in analogy to the preparation of Intermediate C by using 5-bromo-4-fluoro-1H-indole instead of 5-bromo-6-fluoro-1H-indole (compound C2).

### Intermediate I

### cis-tert-butyl 3-ethynylcyclobutanecarboxylate

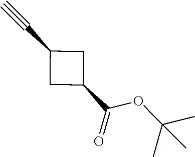

The compound was prepared according to the following scheme:

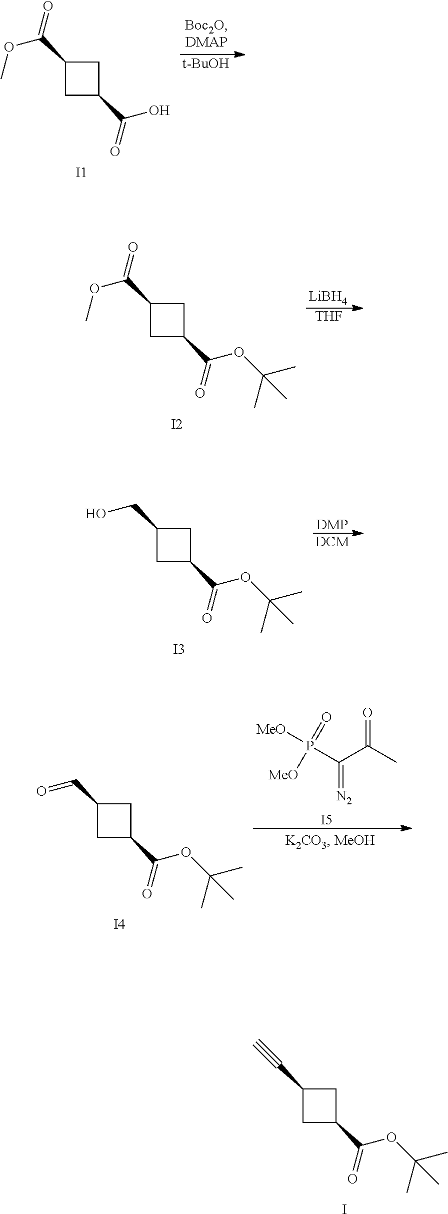

### Step 1: Preparation of cis-O₁-tert-butyl 03-methyl cyclobutane-1,3-dicarboxylate (compound I2)

To a mixture of cis-3-methoxycarbonylcyclobutanecarboxylic acid (compound I1, 25.0 g, 158.08 mmol) and DMAP (38.6 g, 316.16 mmol) in tert-butanol (450 mL) was dropwise added Boc₂O (37.9 g, 173.89 mmol) in tert-butanol (50 mL). After being stirred at 25° C. for 0.5 h, the reaction mixture was diluted with water (150 mL), extracted with EtOAc (150 mL, three times). The combined organic layer was washed with brine (300 mL), dried over Na₂SO₄, filtered and concentrated in vacuo. The resulting residue was purified by silica column (EtOAc in PE=5%˜10%) to afford cis-O₁-tert-butyl O₃-methyl cyclobutane-1,3-dicarboxylate (compound I2, 29.0 g) as colorless liquid. MS calc'd 215.1 (MH⁺), measured 215.1 (MH⁺).

### Step 2: Preparation of cis-tert-butyl 3-(hydroxymethyl)-cyclobutanecarboxylate (compound I3)

To a mixture of cis-O₁-tert-butyl O₃-methyl cyclobutane-1,3-dicarboxylate (compound I2, 29.0 g, 140.02 mmol) in THF (290 mL) was added lithium borohydride (9.2 g, 420.05 mmol) at 0° C. under nitrogen atmosphere. After being stirred at 25° C. for 2 hrs, the reaction mixture were added with EtOAc (800 mL) and water (200 mL) and the layers were separated. The aqueous phase was extracted with EtOAc (300 mL, three times). The combined organic layer was washed with brine (500 mL), dried over Na₂SO₄, filtered, and concentrated under vacuum to give a residue. The residue was purified by silica column (EtOAc in PE=10%˜30%) to afford cis-tert-butyl 3-(hydroxymethyl)-cyclobutanecarboxylate (compound I3, 19 g) as colorless liquid. MS calc'd 186.1 (MH⁺), measured 186.1 (MH⁺).

### Step 3: Preparation of cis-tert-butyl 3-formylcyclobutanecarboxylate (compound I4)

To a mixture of cis-tert-butyl 3-(hydroxymethyl)-cyclobutanecarboxylate (compound I3, 19.0 g, 102.01 mmol) in DCM (230 mL) was added DMAP (51.9 g, 122.42 mmol) at 0° C. After being stirred at 25° C. for 1 h, the reaction mixture was filtered. The filtrate was concentrated in vacuo and purified by silica column (EtOAc in PE=5%˜20%) to afford cis-tert-butyl 3-formylcyclobutanecarboxylate (compound I4, 15.0 g) as colorless oil. MS calc'd 184.1 (MH⁺), measured 184.1 (MH⁺).

### Step 4: Preparation of cis-tert-butyl 3-ethynylcyclobutanecarboxylate (Intermediate I)

To a mixture of cis-tert-butyl 3-formylcyclobutanecarboxylate (compound I4, 15.0 g, 81.42 mmol) and potassium carbonate (22.5 g, 162.84 mmol) in Methanol (200 mL) was added dimethyl (1-diazo-2-oxopropyl)-phosphonate (compound I5, 23.5 g, 122.13 mmol) at 0° C. The mixture was stirred at 25° C. for 3 h. The reaction mixture was concentrated in vacuo to remove solvent and diluted with water (100 mL) and extracted with ethyl acetate (100 mL, twice). The combined organic layer was washed by brine (100 mL), dried over anhydrous sodium sulfate, filtered and concentrated under vacuum to give a residue. The resulting residue was purified by silica gel chromatography to afford cis-tert-butyl 3-ethynylcyclobutanecarboxylate (Intermediate I, 7 g) as colorless oil. MS calc'd 180.1 (MH⁺), measured 180.1 (MH⁺).

### Intermediate J

### (7S,13S)-7-amino-21-ethyl-24-fluoro-(20M)-20-[2-[(1S)-1-methoxyethyl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

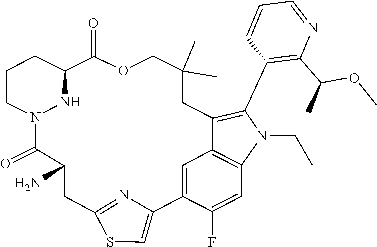

The title compound was prepared in analogy to the preparation of Intermediate G by using 3-bromo-2-[(1S)-1-methoxyethyl]pyridine (compound A1) instead of 4-[5-bromo-6-[(1S)-1-methoxyethyl]-3-pyridyl]morpholine (compound G1).

### Intermediate K

### (7S,13S)-7-amino-24-fluoro-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

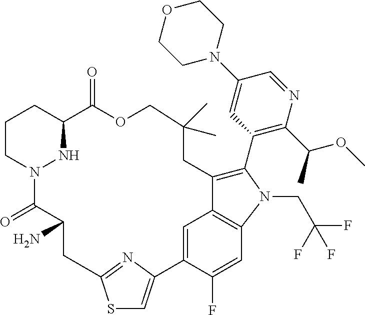

The title compound was prepared in analogy to the preparation of Intermediate G by using 2,2,2-trifluoroethyl trifluoromethanesulfonate instead of iodoethane.

### Example 1

### trans-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide

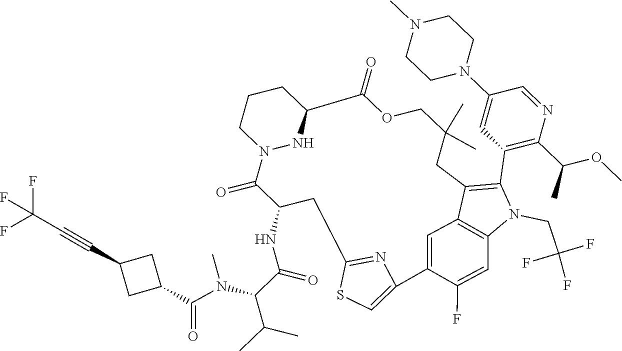

The compound was prepared according to the following scheme:

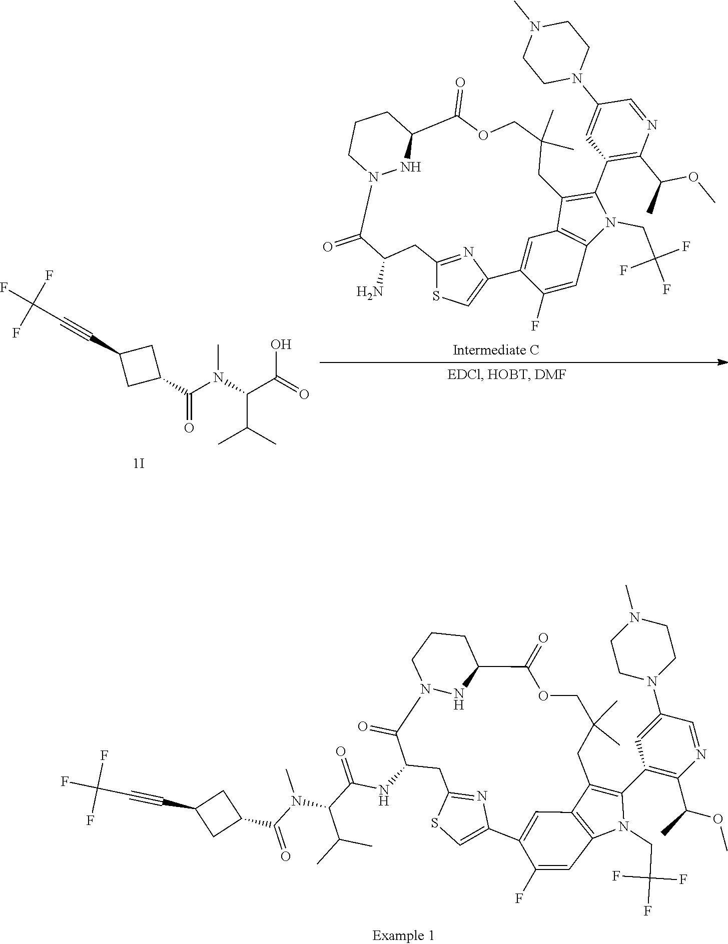

### Step 1: Preparation of trans-N-[(1S)-2-[[(7S,13S)-24-fluoro-(20M)-20-[2-[(1S)-1-methoxyethyl]-5-(4-methylpiperazin-1-yl)-3-pyridyl-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl-2-methyl-propyl]-N-methyl-3-(3,3,3-trifluoroprop-1-ynyl)cyclobutanecarboxamide

To a solution of trans-(2S)-3-methyl-2-[methyl-[3-(3,3,3-trifluoroprop-1-ynyl)cyclobutanecarbonyl]amino]butanoic acid (compound 1I, 70.0 mg, 0.23 mmol), (7S,13S)-7-amino-24-fluoro-(20M)-20-[2-[(1S)-1-methoxy ethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C, 100.0 mg, 0.12 mmol), EDCI (50.0 mg, 0.26 mmol) and DIEA (0.1 mL, 0.62 mmol) in DMF (2 mL) was added HOBT (34.0 mg, 0.25 mmol) at 0° C. After being stirred at 16° C. for 1 h, the reaction mixture was poured into water (20 mL), and extracted with EtOAc (20 mL, three times). The combined organic layer was washed with brine (30 mL), dried over Na₂SO₄, filtered and concentrated under vacuum to give a residue. The resulting residue was purified by prep-HPLC to afford N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide (Example 1, 34.8 mg) as an off-white solid. MS calc'd 1088.5 (MH⁺), measured 1088.5 (MH⁺). ¹H NMR (400 MHz, METHANOL-d₄) 6=8.68 (d, J=7.2 Hz, 1H), 8.50 (d, J=3.2 Hz, 1H), 7.69 (d, J=2.4 Hz, 1H), 7.54-7.43 (m, 2H), 5.72-5.65 (m, 1H), 5.26-5.10 (m, 1H), 4.82-4.75 (m, 1H), 4.46-4.38 (m, 1H), 4.27-4.18 (m, 2H), 4.12-3.86 (m, 2H), 3.82-3.56 (m, 5H), 3.54-3.41 (m, 2H), 3.37-3.33 (m, 4H), 3.30-3.23 (m, 2H), 3.21-3.06 (m, 2H), 3.02-2.91 (m, 6H), 2.87-2.68 (m, 3H), 2.65-2.16 (m, 6H), 2.05-1.55 (m, 4H), 1.45 (d, J=6.0 Hz, 3H), 1.35-1.25 (m, 1H), 1.03-0.94 (m, 5H), 0.91-0.82 (m, 3H), 0.44 (s, 3H) ppm.

The compound 1I was prepared according to the following scheme:

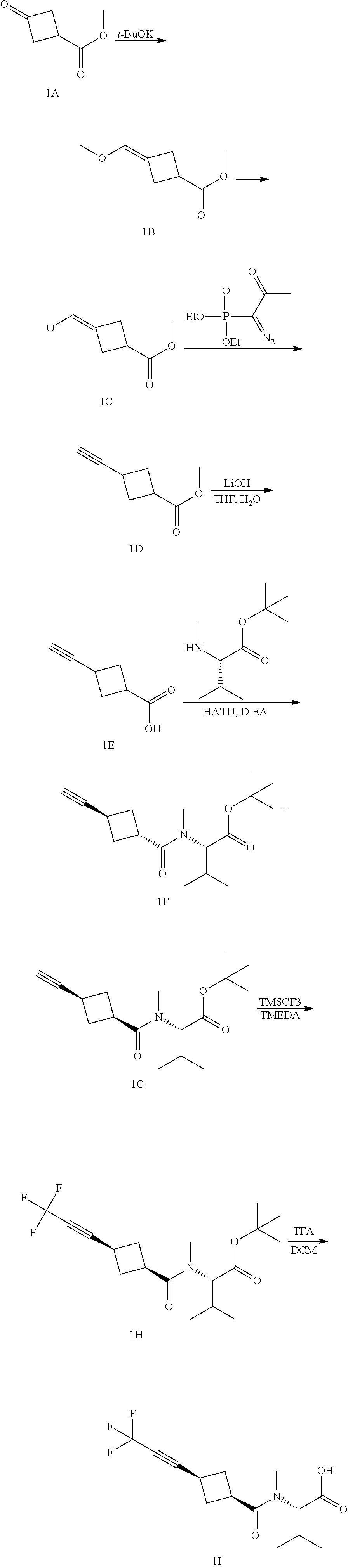

### Step 1: Preparation of methyl 3-(methoxymethylene)cyclobutanecarboxylate (compound 1B)

To a solution of (methoxymethyl)triphenylphosphonium chloride (267.5 g, 780.46 mmol) in THF (1.6 L) was added potassium tert-butoxide (87.6 g, 780.46 mmol) slowly at 0° C. and warmed to 20° C. Methyl 3-oxocyclobutanecarboxylate (compound 1A, 50.0 g, 390.23 mmol) was added to the reaction mixture after 1.5 hrs. After being stirred at 70° C. for 3 hrs, the reaction mixture was concentrated under vacuum to give the residue. A mixed solution of PE in EtOAc (10:1, 1.1 L) was added to the residue. After being stirred at 20° C. for 0.5 h, the suspension was filtered and the filtrate was concentrated under vacuum to give a residue. The residue was purified by column chromatography (EtOAc in PE: 0%-10%) to afford methyl 3-(methoxymethylene)cyclobutanecarboxylate (compound 1B, 18.0 g) as yellow oil. ¹H NMR (400 MHz, CHLOROFORM-d) δ: 5.85-5.79 (m, 1H), 3.70 (s, 3H), 3.60 (s, 3H), 3.29-3.09 (m, 1H), 3.01-2.90 (m, 2H), 2.89-2.71 (m, 2H) ppm.

### Step 2: Preparation of methyl 3-formylcyclobutanecarboxylate (compound 1C)

To a solution of methyl 3-(methoxymethylene)cyclobutanecarboxylate (compound 1B, 26.0 g, 166.47 mmol) in DCM (300 mL) and water (30 mL) was added TFA (26.0 mL). The reaction mixture was stirred at 20° C. for 3 hrs. After the reaction was completed, the reaction mixture was added with H₂O (600 mL) then extracted with DCM (100 mL, three times). The organic layer was washed with brine (500 mL), dried over Na₂SO₄, filtered and concentrated under vacuum to give methyl 3-formylcyclobutanecarboxylate (compound 1C, 18.0 g, 126.63 mmol) as yellow oil. ¹H NMR (400 MHz, CHLOROFORM-d) δ: 9.84-9.52 (m, 1H), 3.75-3.63 (m, 3H), 3.32-3.20 (m, 1H), 3.18-3.07 (m, 1H), 2.67-2.38 (m, 4H) ppm.

### Step 3: Preparation of methyl 3-ethynylcyclobutanecarboxylate (compound 1D)

To a solution of methyl 3-formylcyclobutanecarboxylate (compound 1C, 10.0 g, 70.35 mmol) in methanol (120 mL) was cooled to 0° C. and then dimethyl (1-diazo-2-oxopropyl)phosphonate (21.0 g, 109.31 mmol) and potassium carbonate (20.0 g, 144.71 mmol) were added to the reaction mixture. After being stirred at 20° C. for 3 hrs, the reaction mixture was added with H₂O (150 mL) and then extracted with PE (60 mL, twice). The combined organic layer was washed with brine (80 mL), dried over Na₂SO₄, filtered and concentrated under vacuum to give a residue. The residue was purified by column chromatography (EtOAc in PE: 0% to 25%) to give methyl 3-ethynylcyclobutanecarboxylate (compound 1D, 6.0 g) as colorless oil. ¹H NMR (400 MHz, CHLOROFORM-d) δ: 3.75-3.62 (m, 3H), 3.42-3.21 (m, 1H), 3.07-2.89 (m, 1H), 2.65-2.33 (m, 4H), 2.23-2.17 (m, 1H) ppm.

### Step 4: Preparation of 3-ethynylcyclobutanecarboxylic acid (compound 1E)

To a solution of methyl 3-ethynylcyclobutanecarboxylate (compound 1D, 6.0 g, 43.43 mmol) in THF (10 mL) and water (30 mL) was added lithium hydroxide (3.6 g, 86.86 mmol) at 0° C. and then the solution was stirred at 20° C. for 3 hrs. After the reaction was completed, the reaction mixture was concentrated under vacuum to remove THF then added with H₂O (60 mL) and extracted with MTBE (30 mL). The MTBE phase was discarded and the pH of the aqueous phase was acidified to pH=5 with HCl aq. (1 N, 60 mL) and it was extracted with EtOAc (60 mL, three times). The combined organic layer was washed with brine (80 mL), dried over Na₂SO₄, filtered and concentrated under vacuum to afford 3-ethynylcyclobutanecarboxylic acid (compound 1E, 3.8 g) as colorless oil. ¹H NMR (400 MHz, CHLOROFORM-d) δ: 12.14-9.87 (m, 1H), 3.36-3.15 (m, 1H), 3.10-2.95 (m, 1H), 2.68-2.53 (m, 2H), 2.51-2.35 (m, 2H), 2.22 (dd, J=15.2, 2.4 Hz, 1H) ppm.

### Step 5: Preparation of trans-tert-butyl (2S)-2-[(3-ethynylcyclobutanecarbonyl)-methyl-amino]-3-methyl-butanoate (compound 1F) and cis-tert-butyl (2S)-2-[(3-ethynylcyclobutanecarbonyl)-methyl-amino]-3-methyl-butanoate (compound 1G)

To a solution of 3-ethynylcyclobutanecarboxylic acid (compound 1E, 3.8 g, 30.61 mmol) in DMF (50 mL) was added DIEA (19.0 mL, 114.96 mmol), HATU (14.3 g, 37.48 mmol). After being stirred at 0° C. for 10 min, tert-butyl (2S)-3-methyl-2-(methylamino)butanoate (5.7 g, 30.44 mmol) was added to the reaction mixture. The reaction mixture was stirred at 0° C. for another 1 h. After the reaction was completed, the reaction mixture was added with H₂O (120 mL) then extracted with EtOAc (40 mL, three times). The combined organic layer was washed with brine (60 mL), dried over Na₂SO₄, filtered and concentrated under vacuum to give a residue. The residue was purified by column chromatography (EtOAc in PE: 9%-16%) and prep-HPLC (column: Welch Ultimate XB-CN 250×50×10 μm; mobile phase: Hexane-EtOH (0.1% FA); B %: 1%-20%, 15 min) to afford cis-tert-butyl (2S)-2-[(3-ethynylcyclobutanecarbonyl)-methyl-amino]-3-methyl-butanoate (compound 1G, faster eluted, 3 g) as yellow oil and trans-tert-butyl (2S)-2-[(3-ethynylcyclobutanecarbonyl)-methyl-amino]-3-methyl-butanoate (compound 1F, slower eluted, 2 g) as yellow oil.

cis-tert-butyl (2S)-2-[(3-ethynylcyclobutanecarbonyl)-methyl-amino]-3-methyl-butanoate (compound 1G, Peak 1). MS calc'd 294.2 (MH⁺), measured 294.1 (MH⁺). ¹H NMR (400 MHz, CHLOROFORM-d) δ=4.80 (d, J=10.4 Hz, 0.5 H), 3.58 (d, J=10.8 Hz, 0.5 H), 3.27-3.11 (m, 1H), 3.01-2.91 (m, 1H), 2.87 (d, J=6.8 Hz, 3H), 2.59-2.40 (m, 4H), 2.25-2.12 (m, 2H), 1.45 (s, 9H), 1.00 (dd, J=14.4, 6.4 Hz, 3H), 0.84 (dd, J=6.8, 1.2 Hz, 3H) ppm. Stereochemistry of compound 1G was confirmed by 2D-NMR.

trans-tert-butyl (2S)-2-[(3-ethynylcyclobutanecarbonyl)-methyl-amino]-3-methyl-butanoate (compound 1F, Peak 2). MS calc'd 294.2 (MH⁺), measured 294.1 (MH⁺). ¹H NMR (400 MHz, CHLOROFORM-d) 6=4.80 (d, J=10.4 Hz, 0.5 H), 3.69-3.46 (m, 1.5 H), 3.11-3.01 (m, 1H), 2.88 (d, J=3.2 Hz, 3H), 2.73-2.60 (m, 2H), 2.40-2.27 (m, 2H), 2.25-2.11 (m, 2H), 1.45 (d, J=2.8 Hz, 9H), 1.01 (dd, J=12.0, 6.8 Hz, 3H), 0.84 (dd, J=6.8, 1.6 Hz, 3H) ppm. Stereochemistry of compound 1F was confirmed by 2D-NMR.

### Step 6: Preparation of trans-tert-butyl (2S)-3-methyl-2-[methyl-[3-(3,3,3-trifluoroprop-1-ynyl)cyclobutanecarbonyl]amino]butanoate (compound 1H)

A suspension of CuI (408.9 mg, 2.15 mmol), K₂CO₃ (593.5 mg, 4.29 mmol) and TMEDA (249.5 mg, 2.15 mmol) in DMF (10 mL) was stirred at 25° C. under argon atmosphere for 20 min. TMSCF₃ (407.1 mg, 2.86 mmol) was added to the reaction and the reaction mixture was stirred for 10 min under argon atmosphere. A solution of TMSCF₃ (407.09 mg, 2.86 mmol) and trans-tert-butyl (2S)-2-[(3-ethynylcyclobutanecarbonyl)-methyl-amino]-3-methyl-butanoate (compound 1F, 420.0 mg, 1.43 mmol) in DMF (10 mL) was added into the reaction. The reaction mixture was stirred at 0° C. for 30 min and allowed to warm to 25° C. After being stirred at 25° C. for another 12 hrs, the reaction mixture was added with H₂O (30 mL) then extracted with EtOAc (10 mL, three times). The combined organic layer was washed with brine (50 mL) and dried over Na₂SO₄, filtered and concentrated under vacuum to give a residue. The residue was purified by reversed-phase chromatography and prep-HPLC to afford trans-tert-butyl (2S)-3-methyl-2-[methyl-[3-(3,3,3-trifluoroprop-1-ynyl)cyclobutanecarbonyl]amino]butanoate (compound 1H, 80.0 mg) as yellow oil. MS calc'd 362.2 (MH⁺), measured 362.1 (MH⁺). ¹H NMR (400 MHz, CHLOROFORM-d) δ=4.80 (d, J=10.0 Hz, 0.5 H), 3.62-3.46 (m, 1.5 H), 3.28-3.13 (m, 1H), 2.89 (d, J=4.4 Hz, 3H), 2.82-2.67 (m, 2H), 2.48-2.38 (m, 2H), 2.29-2.15 (m, 1H), 1.46 (d, J=2.8 Hz, 9H), 1.05-0.98 (m, 3H), 0.85 (d, J=6.8 Hz, 3H) ppm.

### Step 7: Preparation of trans-(2S)-3-methyl-2-[methyl-[3-(3,3,3-trifluoroprop-1-ynyl)cyclobutanecarbonyl]amino]butanoic acid (compound 1I)

To a solution of trans-tert-butyl (2S)-3-methyl-2-[methyl-[3-(3,3,3-trifluoroprop-1-ynyl)cyclobutanecarbonyl]amino]butanoate (compound 1H, 80.0 mg, 0.22 mmol) in DCM (1 mL) was added TFA (1.0 mL) and the mixture was stirred at 20° C. for 1 h. After the reaction was completed, the reaction mixture was concentrated under vacuum to afford trans-(2S)-3-methyl-2-[methyl-[3-(3,3,3-trifluoroprop-1-ynyl)cyclobutanecarbonyl]amino]butanoic acid (compound 1I, 80.0 mg) as yellow oil, which was used directly in the next step. MS calc'd 306.0 (MH⁺), measured 306.0 (MH⁺).

### Example 2

### N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide

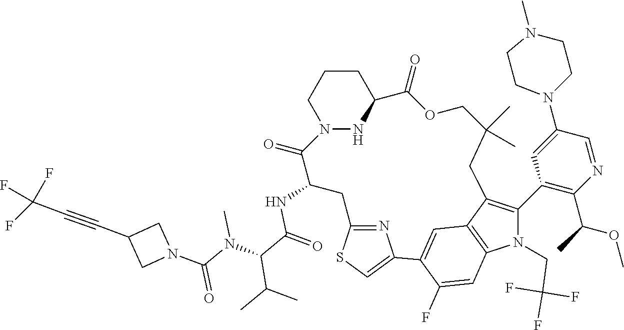

The title compound was prepared in analogy to the preparation of Example 1 by using (2S)-3-methyl-2-[methyl-[3-(3,3,3-trifluoroprop-1-ynyl)azetidine-1-carbonyl]amino]butanoic acid (compound 2E) instead of trans-(2S)-3-methyl-2-[methyl-[3-(3,3,3-trifluoroprop-1-ynyl)cyclobutanecarbonyl]amino]butanoic acid (compound 1I). Example 2 (78.9 mg) was obtained as a yellow solid. MS calc'd 1089.5 (MH⁺), measured 1089.7 (MH⁺). ¹H NMR (400 MHz, Methanol-d₄) δ=8.68 (d, J=7.2 Hz, 1H), 8.50 (d, J=2.8 Hz, 1H), 7.70 (d, J=2.0 Hz, 1H), 7.51 (d, J=2.4 Hz, 1H), 7.48 (d, J=12.8 Hz, 1H), 5.71 (t, J=8.8 Hz, 1H), 5.23-5.13 (m, 1H), 4.47-4.38 (m, 2H), 4.37-4.11 (m, 6H), 4.10-3.93 (m, 3H), 3.81-3.77 (m, 1H), 3.76-3.59 (m, 4H), 3.47 (d, J=14.8 Hz, 1H), 3.35 (s, 3H), 3.29-3.23 (m, 2H), 3.19-3.10 (m, 2H), 2.99 (s, 3H), 2.87 (s, 3H), 2.84-2.79 (m, 1H), 2.57 (d, J=14.4 Hz, 1H), 2.25-2.16 (m, 2H), 2.01-1.92 (m, 1H), 1.87-1.77 (m, 1H), 1.70-1.58 (m, 1H), 1.45 (d, J=6.0 Hz, 3H), 1.39-1.25 (m, 1H), 1.00-0.85 (m, 10H), 0.44 (s, 3H) ppm.

The compound 2E was prepared according to the following scheme:

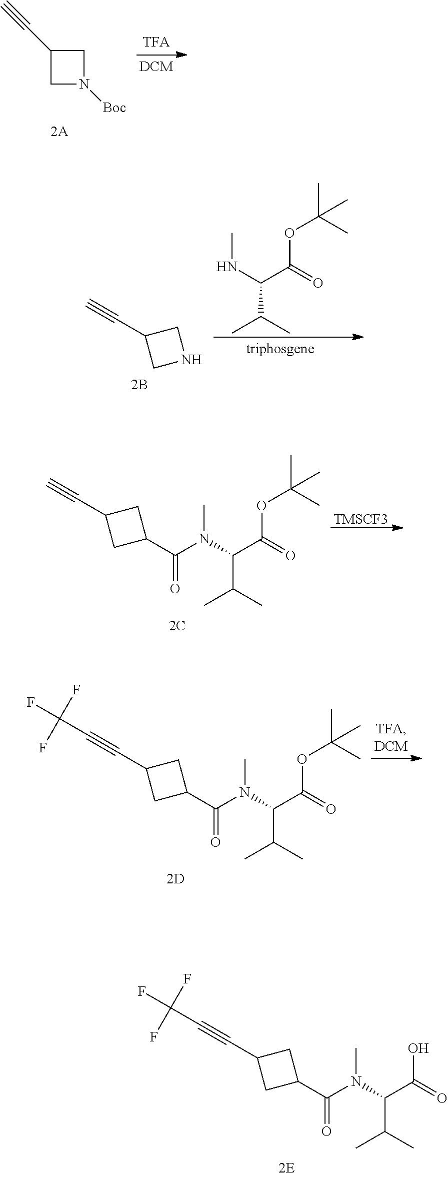

### Step 1: Preparation of 3-ethynylazetidine (compound 2B)

To a solution of tert-butyl 3-ethynylazetidine-1-carboxylate (compound 2A, 3.5 g, 19.31 mmol) in DCM (36 mL) was added TFA (17.7 g, 155.76 mmol). The reaction mixture was stirred at 20° C. for 1 h. After the reaction was completed, the reaction mixture was evaporated, co-evaporated three times with DCM (20 mL) to afford 3-ethynylazetidine (compound 2B, 3.5 g, crude, TFA salt) as yellow oil which was used directly in the next step without purification.

### Step 2: Preparation of tert-butyl (2S)-2-[(3-ethynylazetidine-1-carbonyl)-methyl-amino]-3-methyl-butanoate (compound 2C)

To a mixture of tert-butyl (2S)-3-methyl-2-(methylamino)butanoate (3.7 g, 19.76 mmol) in DCM (50 mL) was added DIEA (8.5 mL, 48.8 mmol) and triphosgene (2.1 g, 7.08 mmol). After being stirred at 0° C. for 10 min, a mixture of 3-ethynylazetidine;2,2,2-trifluoroacetic acid (compound 2B, 3.5 g, 17.94 mmol) and DIEA (13.0 mL, 74.64 mmol) in DCM (50 mL) was added to the reaction. The resulting mixture was stirred at 20° C. for another 1 h. After the reaction was completed, the reaction mixture was added with sat. NaHCO₃ aq. (500 mL) and then extracted with EtOAc (100 mL, twice). The combined organic layer was washed with brine (600 mL), dried over Na₂SO₄, filtered and concentrated under vacuum to give a residue. The residue was purified by column chromatography (EtOAc in PE: 11%-25%) to afford tert-butyl (2S)-2-[(3-ethynylazetidine-1-carbonyl)-methyl-amino]-3-methyl-butanoate (compound 2C, 3.2 g) as yellow oil. MS calc'd 239.2 (M-C₄H₉+H⁺), measured 239.0 (M-C₄H₉+H⁺).

### Step 3: Preparation of tert-butyl (2S)-3-methyl-2-[methyl-[3-(3,3,3-trifluoroprop-1-ynyl)azetidine-1-carbonyl]amino]butanoate (compound 2D)

A mixture of CuI (1.5 g, 8.15 mmol), potassium carbonate (2.3 g, 16.34 mmol) and TMEDA (947.3 mg, 8.15 mmol) in DMF (30 mL) was stirred at 20° C. for 20 min under argon atmosphere. TMSCF₃ (1.5 g, 10.87 mmol) was added to the reaction mixture and then stirred at 20° C. for 20 min. A mixture of tert-butyl (2S)-2-[(3-ethynylazetidine-1-carbonyl)-methyl-amino]-3-methyl-butanoate (compound 2C, 1.6 g, 5.43 mmol) and TMSCF₃ (1.5 g, 10.87 mmol) in DMF (30 mL) was added into the reaction mixture. After being stirred at 20° C. for another 12 hrs under argon atmosphere, the reaction mixture was added with H₂O (100 mL) then extracted with EtOAc (30 mL, three times). The combined organic layer was washed with brine (150 mL), dried over Na₂SO₄, filtered and concentrated under vacuum to give a residue. The residue was purified by column chromatography (EtOAc in PE: 11% to 25%) and concentrated under vacuum to give a residue. The residue was purified again by reversed-phase HPLC to afford tert-butyl (2S)-3-methyl-2-[methyl-[3-(3,3,3-trifluoroprop-1-ynyl)azetidine-1-carbonyl]amino]butanoate (compound 2D, 600.0 mg) as yellow oil. MS calc'd 307.2 (M-C₄H₉+H⁺), measured 307.1 (M-C₄H₉+H⁺).

### Step 4: Preparation of (2S)-3-methyl-2-[methyl-[3-(3,3,3-trifluoroprop-1-ynyl)azetidine-1-carbonyl]amino]butanoic acid (compound 2E)

To a solution of tert-butyl (2S)-3-methyl-2-[methyl-[3-(3,3,3-trifluoroprop-1-ynyl)azetidine-1-carbonyl]amino]butanoate (compound 2D, 300.0 mg, 0.83 mmol) in DCM (2 mL) was added TFA (2.6 g, 23.36 mmol). The reaction mixture was stirred at 20° C. for 1 h. After the reaction was completed, the reaction mixture was concentrated under vacuum to give a residue. The residue was co-evaporated with DCM (6 mL, three times) to afford (2S)-3-methyl-2-[methyl-[3-(3,3,3-trifluoroprop-1-ynyl)azetidine-1-carbonyl]amino]butanoic acid (compound 2E, 250.0 mg) as yellow oil which was used directly in the next step without purification. MS calc'd 307.1 (MH⁺), measured 307.0 (MH⁺).

### Example 3

### cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide

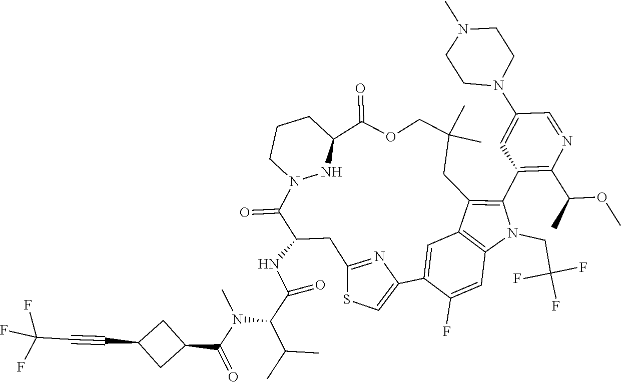

The title compound was prepared in analogy to the preparation of Example 1 by using cis-(2S)-3-methyl-2-[methyl-[3-(3,3,3-trifluoroprop-1-ynyl)cyclobutanecarbonyl]amino]butanoic acid (compound 3B) instead of trans-(2S)-3-methyl-2-[methyl-[3-(3,3,3-trifluoroprop-1-ynyl)cyclobutanecarbonyl]amino]butanoic acid (compound 1I). Example 3 (202.7 mg) was obtained as a yellow solid. MS calc'd 1088.5 (MH⁺), measured 1088.5 (MH⁺). ¹H NMR (400 MHz, Methanol-d₄) δ=8.68 (d, J=7.6 Hz, 1H), 8.50 (d, J=2.8 Hz 1H), 8.67 (d, J=2.4 Hz 1H), 7.52-7.43 (m, 2H), 5.74-5.61 (m, 1H), 5.21-5.12 (m, 1H), 4.94-4.86 (m, 2H), 4.83-4.81 (m, 1H), 4.78 (d, J=11.2 Hz, 1H), 4.46-4.39 (m, 1H), 4.26-4.19 (m, 2H), 4.11-3.90 (m, 2H), 3.84-3.64 (m, 4H), 3.62-3.43 (m, 4H), 3.38-3.33 (m, 4H), 3.18-3.08 (m, 2H), 3.00 (s, 3H), 2.98-2.93 (m, 3H), 2.84-2.75 (m, 1H), 2.74-2.63 (m, 2H), 2.61-2.44 (m, 3H), 2.27-2.16 (m, 2H), 2.01-1.93 (m, 1H), 1.86-1.76 (m, 1H), 1.70-1.59 (m, 1H), 1.45 (d, J=6.0 Hz, 3H), 1.10-1.00 (m, 1H), 1.00-0.95 (m, 5H), 0.90-0.83 (m, 3H), 0.44 (s, 3H) ppm.

Compound 3B was prepared in analogy to the preparation of compound 1I by using cis-tert-butyl (2S)-2-[(3-ethynylcyclobutanecarbonyl)-methyl-amino]-3-methyl-butanoate (compound 1G) instead of trans-tert-butyl (2S)-2-[(3-ethynylcyclobutanecarbonyl)-methyl-amino]-3-methyl-butanoate (compound 1F).

### Example 4

### N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)benzamide

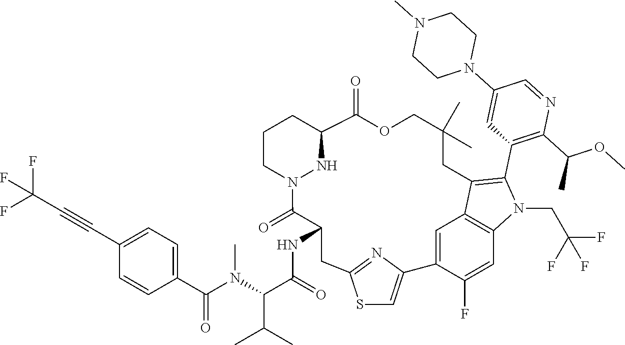

The compound was prepared according to the following scheme:

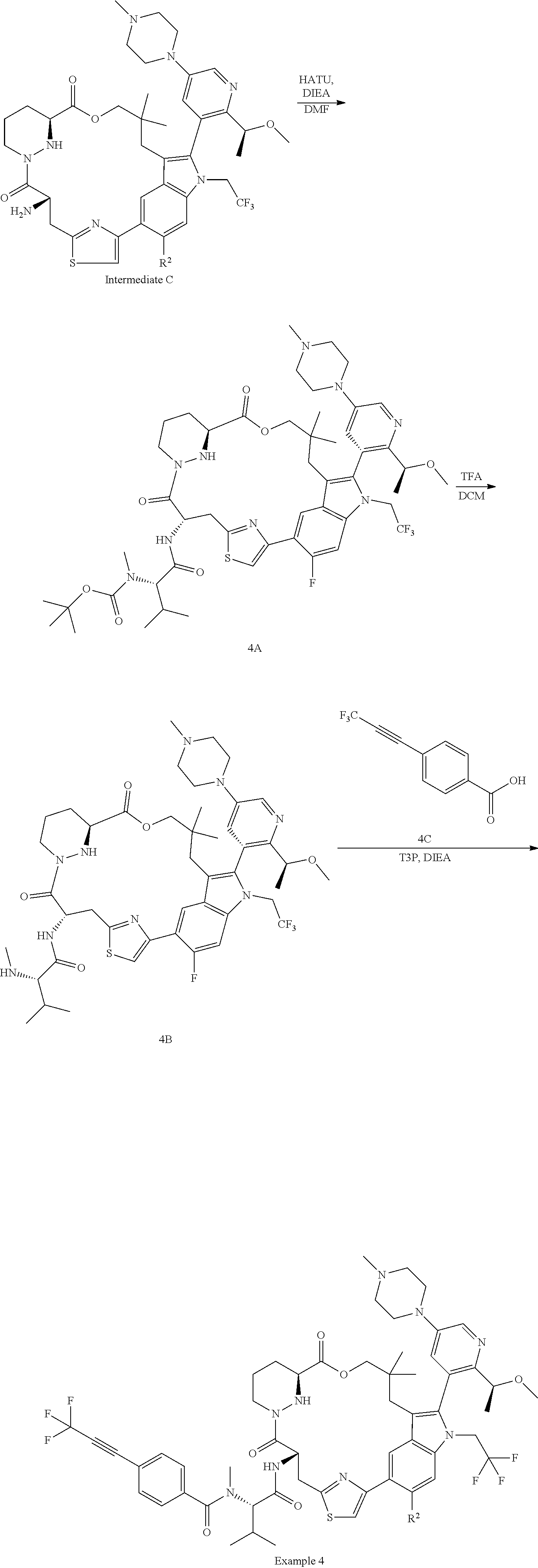

### Step 1: Preparation of tert-butyl N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-carbamate (compound 4A)

To a mixture of BOC-N-ME-VAL-OH (93.8 mg, 0.41 mmol) and DIEA (0.2 mL, 0.94 mmol) in DMF (1 mL) was added HATU (154.3 mg, 0.41 mmol) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C, 250.0 mg, 0.31 mmol). After being stirred at 15° C. for 1 h, the reaction mixture was purified by reversed phase chromatography to afford tert-butyl N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-carbamate (compound 4A, 300.0 mg) as a yellow solid. MS calc'd 1014.4 (MH⁺), measured 1014.3 (MH⁺).

### Step 2: Preparation of (2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-(methylamino)butanamide (compound 4B)

To a mixture of tert-butyl N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-carbamate (compound 4A, 300.0 mg, 0.3 mmol) in DCM (6 mL) was added TFA (4.0 mL). After being stirred at 15° C. for 0.5 h, the reaction mixture was concentrated in vacuo to get a residue. The resulting residue was diluted with sat. NaHCO₃ aq. (30 mL), extracted with EtOAc (20 mL, three times). The combined organic layer was washed with brine (30 mL), dried over Na₂SO₄, filtered and concentrated in vacuo to afford (2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-(methylamino)butanamide (compound 4B, 270.0 mg) as yellow oil, which was used directly in the next step. MS calc'd 914.4 (MH⁺), measured 914.3 (MH⁺).

### Step 3: Preparation of N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)benzamide (Example 4)

To a solution of (2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-(methylamino)butanamide (compound 4B, 210 mg, 0.23 mmol) and 4-(3,3,3-trifluoroprop-1-ynyl)benzoic acid (compound 4C, 49.2 mg, 0.23 mmol) in DMF (0.5 mL) was added DIEA (0.1 mL, 0.38 mmol) and T₃P (146.2 mg, 0.23 mmol). After being stirred at 20° C. for 0.5 hour, the reaction mixture was added into water (40 mL) and extracted with EtOAc (30 mL, three times). The combined organic layer was washed by brine (30 mL, three times), dried over anhydrous sodium sulfate, filtered and concentrated under vacuum to give a residue, which was purified by prep-HPLC to afford N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)benzamide (Example 4, 9.8 mg) as an off-white solid. MS calc'd 1110.4 (MH⁺), measured 1110.6 (MH⁺). ¹H NMR (400 MHz, Methanol-d₄) δ=8.69 (d, J=9.2 Hz, 1H), 8.47-8.43 (m, 1H), 7.79 (d, J=8.4 Hz, 2H), 7.73-7.69 (m, 1H), 7.58 (d, J=8.0 Hz, 2H), 7.49-7.44 (m, 1H), 7.39-7.33 (m, 1H), 5.78-5.71 (m, 1H), 5.19-5.10 (m, 1H), 4.60-4.41 (m, 1H), 4.32-4.15 (m, 2H), 3.80-3.70 (m, 1H), 3.53-3.40 (m, 5H), 3.23-3.08 (m, 4H), 2.97 (s, 3H), 2.95-2.90 (m, 3H), 2.84-2.71 (m, 2H), 2.62-2.52 (m, 4H), 2.46-2.14 (m, 3H), 2.08-1.53 (m, 4H), 1.43 (d, J=6.0 Hz, 3H), 1.29 (s, 3H), 1.06 (t, J=6.4 Hz, 5H), 0.97 (s, 3H), 0.48-0.41 (m, 3H) ppm.

The compound 4B was prepared according to the following scheme:

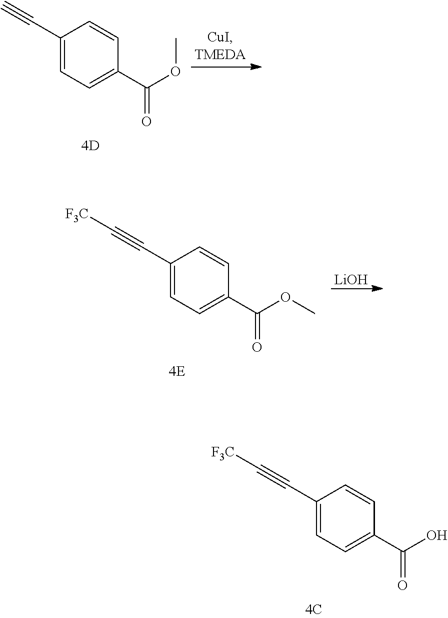

### Step 1: Preparation of methyl 4-(3,3,3-trifluoroprop-1-ynyl)benzoate (compound 8B)

A suspension of copper(I) iodide (1.8 g, 9.37 mmol), potassium carbonate (2.6 g, 18.77 mmol), TMEDA (1.1 g, 9.37 mmol) in DMF (10 mL) was stirred at 20° C. under argon atmosphere. After being stirred for 20 min, the reaction mixture was added with TMSCF₃ (1.8 g, 12.49 mmol). A mixture of methyl 4-ethynylbenzoate (compound 4D, 1.0 g, 6.24 mmol) and TMSCF₃ (1.8 g, 12.49 mmol) in DMF (10 mL) was added slowly to the reaction mixture and then it was stirred at 20° C. for another 12 hrs under argon atmosphere. After the reaction was completed, the reaction mixture was added with H₂O (200 mL) and extracted with EtOAc (80 mL, three times). The combined organic layer was washed with brine (80 mL), dried over Na₂SO₄, filtered and concentrated under vacuum to give a residue. The resulting residue was purified by column chromatography (SiO₂, EtOAc in PE: 0%-95%) to afford methyl 4-(3,3,3-trifluoroprop-1-ynyl)benzoate (compound 4D, 233.0 mg) as a colorless solid. ¹H NMR (400 MHz, CHLOROFORM-d) δ=8.08 (d, J=8.4 Hz, 2H), 7.64 (d, J=8.4 Hz, 2H), 3.95 (s, 3H) ppm.

### Step 2: Preparation of 4-(3,3,3-trifluoroprop-1-ynyl)benzoic acid (compound 4C)

To a solution of methyl 4-(3,3,3-trifluoroprop-1-ynyl)benzoate (compound 4D, 180.0 mg, 0.79 mmol) in THF (1 mL), water (1 mL) was added lithium hydroxide monohydrate (69.5 mg, 1.66 mmol). The mixture was stirred at 20° C. for 1 hour. After the reaction was completed, the pH of the reaction mixture was adjusted to 6 with 1M HCl aqueous solution and it was extracted with EtOAc (10 mL, three times). The combined organic layer was concentrated under vacuum to afford 4-(3,3,3-trifluoroprop-1-ynyl)benzoic acid (compound 4C, 168.0 mg) as a white solid, which was used in the next step directly.

### Example 5

### cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(2-pyrimidin-2-ylethynyl)cyclobutanecarboxamide

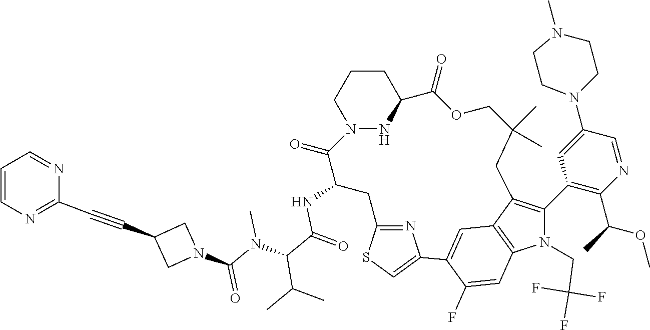

The title compound was prepared in analogy to the preparation of Example 1 by using (2S)-3-methyl-2-[methyl-[cis-3-(2-pyrimidin-2-ylethynyl)cyclobutanecarbonyl]amino]butanoic acid (compound 5B) instead of trans-(2S)-3-methyl-2-[methyl-[3-(3,3,3-trifluoroprop-1-ynyl)cyclobutanecarbonyl]amino]butanoic acid (compound 1I). Example 4 (1.3 mg) was obtained as a yellow solid. MS calc'd 1098.5 (MH⁺), measured 1098.5 (MH⁺). ¹H NMR (400 MHz, CHLOROFORM-d) δ=8.74-8.64 (m, 2H), 8.52 (d, J=2.8 Hz, 1H), 7.72-7.61 (m, 1H), 7.55-7.46 (m, 1H), 7.25-7.20 (m, 1H), 7.18-7.09 (m, 2H), 5.74-5.62 (m, 1H), 5.13-4.88 (m, 1H), 4.79-4.68 (m, 1H), 4.63-4.41 (m, 2H), 4.35-4.16 (m, 2H), 4.13-3.93 (m, 1H), 3.89-3.77 (m, 2H), 3.77-3.69 (m, 2H), 3.66-3.53 (m, 3H), 3.52-3.46 (m, 1H), 3.39-3.33 (m, 3H), 3.32-3.21 (m, 2H), 3.20-3.07 (m, 3H), 2.92-2.87 (m, 3H), 2.87-2.82 (m, 2H), 2.80 (s, 1H), 2.73-2.61 (m, 3H), 2.43-2.37 (m, 1H), 2.36-2.29 (m, 1H), 2.26-2.20 (m, 1H), 2.14 (br s, 1H), 2.05-2.00 (m, 1H), 1.98-1.89 (m, 4H), 1.60-1.57 (m, 1H), 1.49-1.45 (m, 3H), 1.38-1.35 (m, 1H), 1.28-1.25 (m, 3H), 1.17 (br dd, J=6.9, 19.4 Hz, 2H), 1.06 (br d, J=6.5 Hz, 2H), 1.11-0.76 (m, 5H) ppm.

The compound 5B was prepared according to the following scheme:

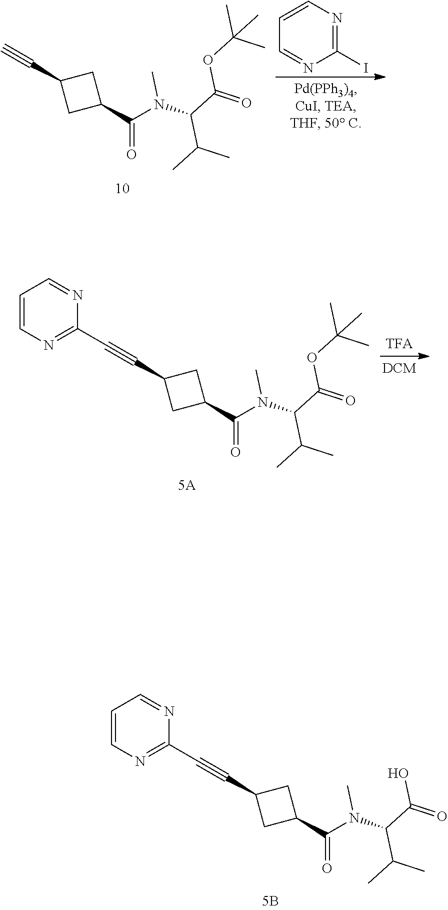

### Step 1: Preparation of tert-butyl (2S)-3-methyl-2-[methyl-[cis 3-(2-pyrimidin-2-ylethynyl)cyclobutanecarbonyl]amino]butanoate (compound 5A)

To a solution of cis-tert-butyl (2S)-2-[(3-ethynylcyclobutanecarbonyl)-methyl-amino]-3-methyl-butanoate (compound 1G, 100.0 mg, 0.34 mmol) in THF (1 mL) were added triethylamine (0.2 mL, 1.02 mmol), 2-iodopyrimidine (70.2 mg, 0.34 mmol), tetrakis(triphenylphosphine)palladium(0) (39.4 mg, 0.03 mmol) and CuI (6.5 mg, 0.03 mmol). The reaction mixture was degassed and purged with nitrogen for three times and stirred at 50° C. for 1 h. After the reaction was completed, the reaction mixture was added with H₂O (60 mL) then extracted with EtOAc (40 mL, twice). The combined organic layer was washed with brine (60 mL), dried over Na₂SO₄, filtered and concentrated under vacuum to give a residue. The residue was purified by prep-HPLC to afford tert-butyl (2S)-3-methyl-2-[methyl-[cis 3-(2-pyrimidin-2-ylethynyl)cyclobutanecarbonyl]amino]butanoate (compound 5A, 60 mg) as yellow oil. MS calc'd 372 (MH⁺), measured 372 (MH⁺).

### Step 2: Preparation of (2S)-3-methyl-2-[methyl-[cis-3-(2-pyrimidin-2-ylethynyl)cyclobutanecarbonyl]amino]butanoic acid (compound 5B)

To a solution of tert-butyl (2S)-3-methyl-2-[methyl-[cis 3-(2-pyrimidin-2-ylethynyl)cyclobutanecarbonyl]amino]butanoate (compound 5A, 60.0 mg, 0.16 mmol) in DCM (1 mL) was added TFA (0.2 mL). After being stirred at 20° C. for 1 h, the reaction mixture was concentrated in vacuo to give (2S)-3-methyl-2-[methyl-[cis-3-(2-pyrimidin-2-ylethynyl)cyclobutanecarbonyl]amino]butanoic acid (compound 5B, 50 mg). The crude was used in the next step. MS calc'd 316 (MH⁺), measured 316 (MH⁺).

### Example 6

### cis-N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide

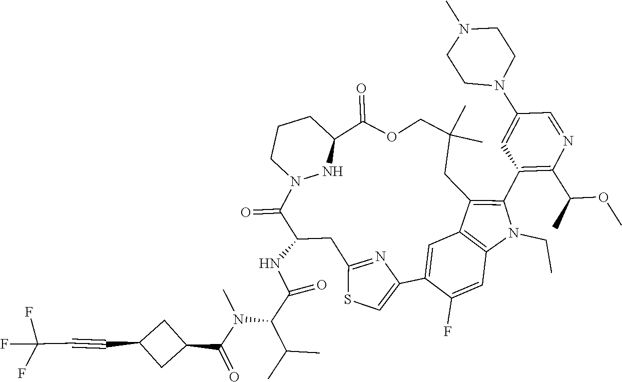

The title compound was prepared in analogy to the preparation of Example 1 by using cis-(2S)-3-methyl-2-[methyl-[3-(3,3,3-trifluoroprop-1-ynyl)cyclobutanecarbonyl]amino]butanoic acid (compound 3B) and (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate D) instead of trans-(2S)-3-methyl-2-[methyl-[3-(3,3,3-trifluoroprop-1-ynyl)cyclobutanecarbonyl]amino]butanoic acid (compound 1I) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 6 (32.2 mg) was obtained as a yellow solid. MS calc'd 1034.5 (MH⁺), measured 1034.5 (MH⁺). ¹H NMR (400 MHz, Methanol-d₄) δ=8.66 (d, J=7.6 Hz, 1H), 8.48 (d, J=2.8 Hz, 1H), 7.62 (d, J=2.4 Hz, 1H), 7.52 (d, J=2.8 Hz, 1H), 7.33 (d, J=12.8 Hz, 1H), 5.78-5.67 (m, 1H), 4.78-4.74 (m, 2H), 4.48-4.36 (m, 1H), 4.33-4.26 (m, 1H), 4.24-3.96 (m, 4H), 3.81-3.64 (m, 3H), 3.56-3.42 (m, 3H), 3.34 (s, 4H), 3.29-3.24 (m, 3H), 3.12-2.89 (m, 8H), 2.89-2.80 (m, 1H), 2.80-2.31 (m, 6H), 2.31-2.12 (m, 2H), 1.98-1.87 (m, 1H), 1.87-1.75 (m, 1H), 1.75-1.54 (m, 1H), 1.43 (d, J=6.0 Hz, 3H), 1.08-0.88 (m, 9H), 0.85 (d, J=6.4 Hz, 3H), 0.58-0.41 (m, 3H) ppm.

### Example 7

### cis-N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide

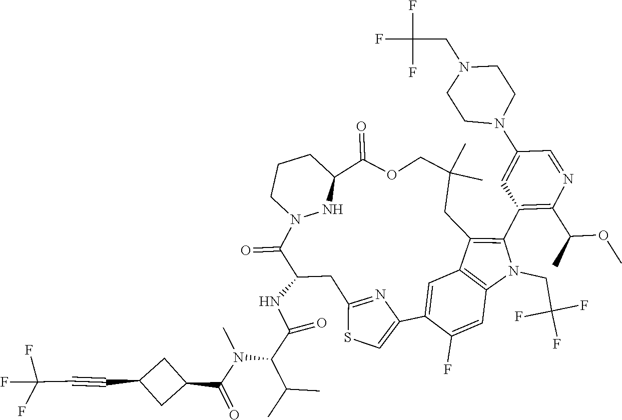

The title compound was prepared in analogy to the preparation of Example 1 by using cis-(2S)-3-methyl-2-[methyl-[3-(3,3,3-trifluoroprop-1-ynyl)cyclobutanecarbonyl]amino]butanoic acid (compound 3B) and (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E) instead of trans-(2S)-3-methyl-2-[methyl-[3-(3,3,3-trifluoroprop-1-ynyl)cyclobutanecarbonyl]amino]butanoic acid (compound 1I) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 7 (30.8 mg) was obtained as a white solid. MS calc'd 1156.5 (MH⁺), measured 1157.1 (MH). ¹H NMR (400 MHz, Methanol-d₄) (=8.71 (d, J=7.6 Hz, 1H), 8.41 (d, J=2.8 Hz, 1H), 7.67 (d, J=2.4 Hz, 1H), 7.58 (s, 1H), 7.47 (d, J=12.4 Hz, 1H), 5.70 (t, J=8.8 Hz, 1H), 5.24-5.06 (m, 2H), 4.80-4.74 (m, 2H), 4.48-4.35 (m, 1H), 4.30-4.15 (m, 2H), 3.80-3.69 (m, 2H), 3.62-3.47 (m, 2H), 3.47-3.39 (m, 5H), 3.38-3.35 (m, 3H), 3.22-3.11 (m, 3H), 2.98-2.82 (m, 8H), 2.72-2.60 (m, 3H), 2.51-2.46 (m, 1H), 2.28-2.16 (m, 2H), 2.01-1.91 (m, 1H), 1.89-1.74 (m, 1H), 1.69-1.58 (m, 1H), 1.46 (d, J=6.0 Hz, 3H), 1.10-1.01 (m, 1H), 1.01-0.83 (m, 9H), 0.49 (s, 3H) ppm.

### Example 8

### cis-N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide

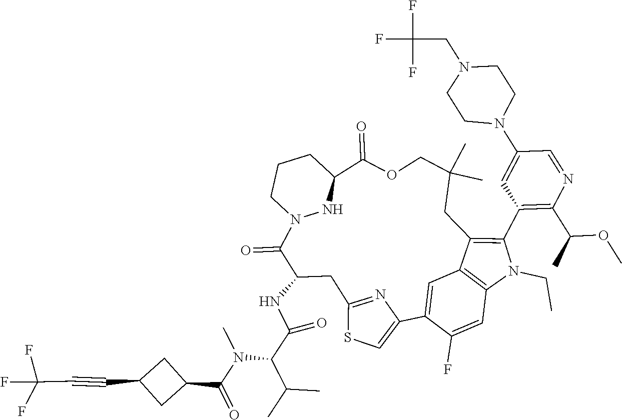

The title compound was prepared in analogy to the preparation of Example 1 by using cis-(2S)-3-methyl-2-[methyl-[3-(3,3,3-trifluoroprop-1-ynyl)cyclobutanecarbonyl]amino]butanoic acid (compound 3B) and (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate F) instead of trans-(2S)-3-methyl-2-[methyl-[3-(3,3,3-trifluoroprop-1-ynyl)cyclobutanecarbonyl]amino]butanoic acid (compound 1I) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 8 (23.3 mg) was obtained as a white solid. MS calc'd 1102.5 (MH⁺), measured 1102.7 (MH⁺). ¹H NMR (400 MHz, Methanol-d₄) δ=8.65 (d, J=7.2 Hz, 1H), 8.40 (d, J=2.8 Hz, 1H), 7.61 (d, J=2.4 Hz, 1H), 7.33-7.27 (m, 2H), 5.90-5.68 (m, 1H), 4.89 (s, 1H), 4.83-4.82 (m, 1H), 4.77 (d, J=11.2 Hz, 1H), 4.47-4.37 (m, 1H), 4.26-4.12 (m, 4H), 3.82-3.42 (m, 5H), 3.36-3.33 (m, 4H), 3.27 (s, 1H), 3.17-3.11 (m, 2H), 3.02 (d, J=14.4 Hz, 1H), 2.97-2.91 (m, 3H), 2.90-2.81 (m, 5H), 2.74-2.60 (m, 3H), 2.60-2.36 (m, 2H), 2.29-2.12 (m, 2H), 1.99-1.91 (m, 1H), 1.88-1.75 (m, 1H), 1.69-1.57 (m, 1H), 1.42 (d, J=6.0 Hz, 3H), 1.17-0.88 (m, 10H), 0.85 (d, J=6.4 Hz, 3H), 0.50 (s, 3H) ppm.

### Example 9

### trans-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)cyclohexanecarboxamide

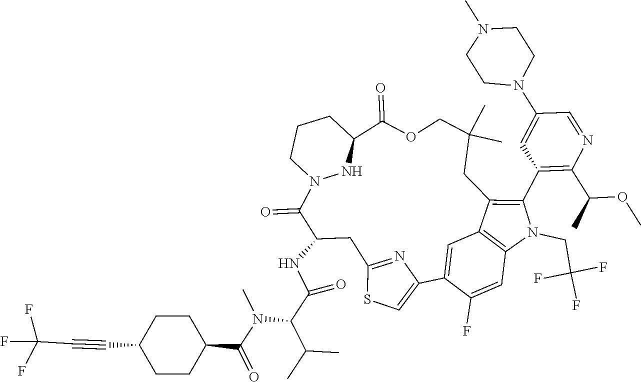

The title compound was prepared in analogy to the preparation of Example 4 by using trans 4-(3,3,3-trifluoroprop-1-ynyl)cyclohexanecarboxylic acid (compound 9g) instead of 4-(3,3,3-trifluoroprop-1-ynyl)benzoic acid (compound 4C). Example 9 (30.8 mg) was obtained as a white solid. MS calc'd 1116.5 (MH⁺), measured 1116.7 (MH⁺). ¹H NMR (400 MHz, Methanol-d₄) δ=8.68 (d, J=7.2 Hz, 1H), 8.53-8.48 (m, 1H), 7.70 (d, J=2.4 Hz, 1H), 7.51-7.45 (m, 2H), 5.68 (t, J=8.4 Hz, 1H), 5.21-5.14 (m, 1H), 4.80 (d, J=10.8 Hz, 1H), 4.46-4.37 (m, 1H), 4.25-4.18 (m, 2H), 4.15-3.87 (m, 2H), 3.81-3.76 (m, 1H), 3.73-3.64 (m, 2H), 3.45 (d, J=14.8 Hz, 1H), 3.35 (s, 3H), 3.27-3.23 (m, 1H), 3.16 (s, 1H), 3.09 (s, 3H), 3.00 (s, 3H), 2.88-2.76 (m, 2H), 2.61-2.53 (m, 2H), 2.26-1.84 (m, 12H), 1.66-1.56 (m, 4H), 1.50-1.43 (m, 6H), 1.02-0.83 (m, 9H), 0.44 (s, 3H) ppm.

The compound 9g was prepared according to the following scheme:

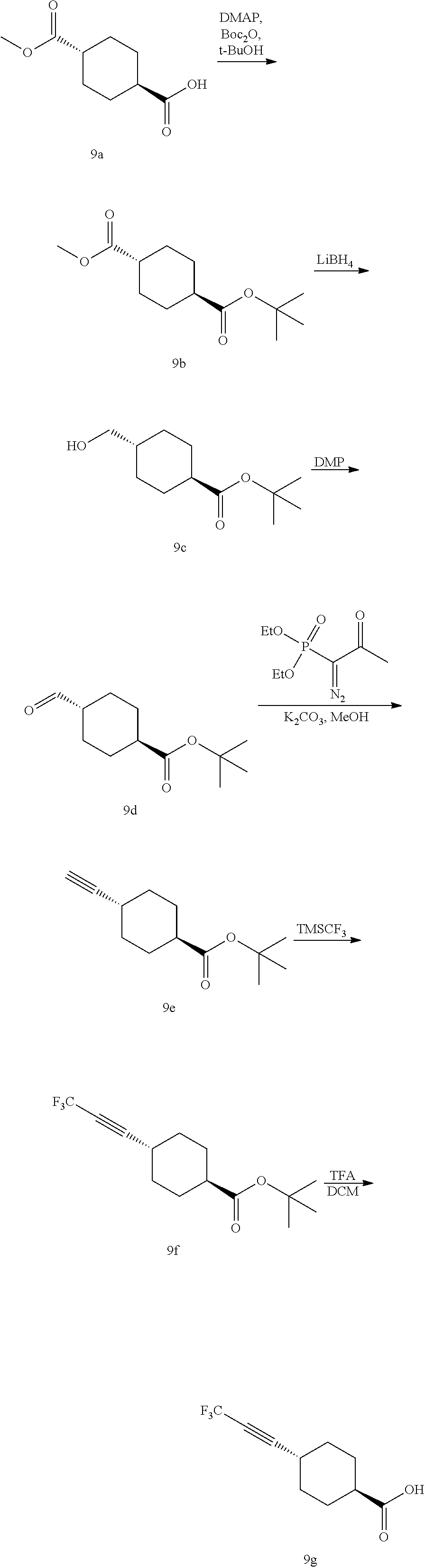

### Step 1: Preparation of trans 4-tert-butyl 1-methyl cyclohexane-1,4-dicarboxylate (compound 9b)

To a solution of trans 4-methoxycarbonylcyclohexanecarboxylic acid (compound 9a, 5.0 g, 26.85 mmol) in tert-butanol (100 mL) was added 4-dimethylaminopyridine (6.6 g, 53.7 mmol), then di-t-butyldicarbonate (6.5 g, 29.54 mmol) was slowly added to the reaction mixture at 20° C. The reaction mixture was stirred at 20° C. for 2 hrs. After the reaction was completed, the reaction mixture was added with H₂O (500 mL), and extracted with EtOAc (80 mL, three times). The combined organic layer was washed with brine (400 mL), dried over Na₂SO₄, filtered and concentrated under vacuum to give a residue. The residue was purified by column chromatography (EtOAc in PE: 0%-10%) to afford trans 4-tert-butyl 1-methyl cyclohexane-1,4-dicarboxylate (compound 9b, 6.5 g) as a white solid. ¹H NMR (400 MHz, CHLOROFORM-d) δ=3.67 (s, 3H), 2.32-2.23 (m, 1H), 2.21-2.12 (m, 1H), 2.05-1.96 (m, 4H), 1.44-1.42 (m, 13H) ppm.

### Step 2: Preparation of trans tert-butyl 4-(hydroxymethyl)cyclohexanecarboxylate (compound 9c)

To a solution of trans 4-tert-butyl 1-methyl cyclohexane-1,4-dicarboxylate (compound 9b, 5.0 g, 20.63 mmol) in THF (100 mL) was added LiBH₄ (1.4 g, 64.28 mmol) slowly at 0° C. The reaction mixture was stirred at 20° C. for another 12 hrs. After the reaction was completed, it was quenched with H₂O (500 mL) slowly at 0° C., and the reaction mixture was extracted with EtOAc (100 mL, three times). The combined organic layer was washed with brine (600 mL), dried over Na₂SO₄, filtered and concentrated under vacuum to give a residue. The residue was purified by column chromatography (EtOAc in PE: 10%-30%) to afford trans tert-butyl 4-(hydroxymethyl)cyclohexanecarboxylate (compound 9c, 3.8 g) as yellow oil. ¹H NMR (400 MHz, CHLOROFORM-d) δ: 3.47 (d, J=6.0 Hz, 2H), 2.18-2.08 (m, 1H), 2.03-1.94 (m, 2H), 1.91-1.79 (m, 2H), 1.58-1.26 (m, 14H) ppm.

### Step 3: Preparation of trans tert-butyl 4-formylcyclohexanecarboxylate (compound 9d)

To a solution of trans tert-butyl 4-(hydroxymethyl)cyclohexanecarboxylate (compound 9c, 3.8 g, 17.73 mmol) in DCM (100 mL) was added DMP (11.28 g, 26.6 mmol) slowly at 0° C. The reaction mixture was stirred at 20° C. for 1 h. After the reaction was completed, the reaction mixture was concentrated under vacuum to remove DCM directly to give a residue. The residue was purified by column chromatography (EtOAc in PE=10% to 30%) and concentrated under vacuum to give trans tert-butyl 4-formylcyclohexanecarboxylate (compound 9d, 2.5 g) as yellow oil. ¹H NMR (400 MHz, CHLOROFORM-d) δ=9.63 (d, J=1.2 Hz, 1H), 2.33-1.93 (m, 6H), 1.59-1.20 (m, 13H) ppm.

### Step 4: Preparation of trans tert-butyl 4-ethynylcyclohexanecarboxylate (compound 9e)

To a solution of trans tert-butyl 4-formylcyclohexanecarboxylate (compound 9d, 2.5 g, 11.78 mmol) in methanol (50 mL) was added potassium carbonate (3.5 g, 25.32 mmol). The reaction mixture was cooled to 0° C., and it was added with dimethyl (1-diazo-2-oxopropyl)phosphonate (3.5 g, 18.22 mmol) slowly. The reaction mixture was stirred at 20° C. for another 3 hrs. After the reaction was completed, the reaction mixture was added with H₂O (200 mL) and then extracted with PE (60 mL, twice). The combined organic layer was washed with brine (800 mL), dried over Na₂SO₄, filtered and concentrated under vacuum to give a residue. The residue was purified by column chromatography (EtOAc in PE: 0%-16%) to afford trans tert-butyl 4-ethynylcyclohexanecarboxylate (compound 9e, 1.5 g) as a white solid. ¹H NMR (400 MHz, CHLOROFORM-d) δ=2.29-1.94 (m, 7H), 1.49-1.34 (s, 13H) ppm.

### Step 5: Preparation of trans tert-butyl 4-(3,3,3-trifluoroprop-1-ynyl)cyclohexanecarboxylate (compound 9f)

To a solution of TMEDA (450 mg, 3.87 mmol) in DMF (15 mL) was added CuI (740 mg, 3.6 mmol) and potassium carbonate (1 g, 7.24 mmol) at 25° C. After being stirred vigorously at 25° C. for 20 min, the reaction mixture was added with TMSCF₃ (700 mg, 4.92 mmol) under argon atmosphere, then stirred at 25° C. for another 20 min. The reaction mixture was cooled to 0° C. and it was added with a mixture of trans tert-butyl 4-ethynylcyclohexanecarboxylate (compound 9e, 500.0 mg, 2.4 mmol) and TMSCF₃ (700 mg, 4.92 mmol) in DMF (10 mL). The reaction mixture was stirred at 0° C. for 30 min and it was allowed to warm to 25° C. for 12 hrs. After the reaction was completed, the reaction mixture was poured into water (60 mL) and extracted with ethyl acetate (50 mL, three times). The combined organic layer was washed by brine (80 mL, three times), dried over anhydrous sodium sulfate, filtered and concentrated under vacuum to give a residue, which was purified by column chromatography (EtOAc in PE: 0%-10%) to afford trans tert-butyl 4-(3,3,3-trifluoroprop-1-ynyl)cyclohexanecarboxylate (compound 9f, 500 mg, 1.48 mmol) as a white solid. ¹H NMR (400 MHz, CHLOROFORM-d) δ=2.17-1.94 (m, 6H), 1.48-1.34 (m, 13H) ppm.

### Step 6: Preparation of trans 4-(3,3,3-trifluoroprop-1-ynyl)cyclohexanecarboxylic acid (compound 9g)

To a solution of trans tert-butyl 4-(3,3,3-trifluoroprop-1-ynyl)cyclohexanecarboxylate (compound 9f, 100.0 mg, 0.36 mmol) in DCM (1 mL) was added with TFA (1.0 mL). The reaction mixture was stirred at 20° C. for 0.5 h. After the reaction was completed, the reaction mixture was concentrated under vacuum to give a residue. The residue was co-evaporated with DCM (5 mL) three times to afford trans 4-(3,3,3-trifluoroprop-1-ynyl)cyclohexanecarboxylic acid (compound 9g, 50.0 mg) as white solid, which was used directly in the next step without purification.

### Example 10

### cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)cyclohexanecarboxamide

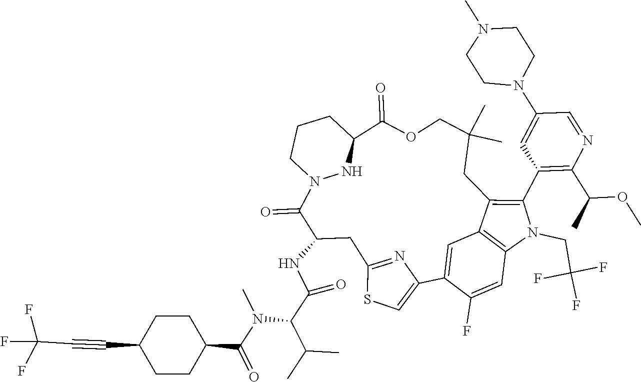

The title compound was prepared in analogy to the preparation of Example 4 by using cis 4-(3,3,3-trifluoroprop-1-ynyl)cyclohexanecarboxylic acid (compound 10g) instead of 4-(3,3,3-trifluoroprop-1-ynyl)benzoic acid (compound 4C). Example 10 (39.7 mg) was obtained as a white solid. MS calc'd 1116.5 (MH⁺), measured 1116.4 (MH⁺). ¹H NMR (400 MHz, Methanol-d₄) δ=8.74-8.65 (m, 1H), 8.52-8.49 (m, 1H), 7.72-7.64 (m, 1H), 7.53-7.42 (m, 2H), 5.75-5.61 (m, 1H), 5.26-5.12 (m, 1H), 4.99-4.92 (m, 1H), 4.50-4.35 (m, 1H), 4.28-4.17 (m, 2H), 4.17-3.88 (m, 2H), 3.82-3.74 (m, 1H), 3.73-3.51 (m, 3H), 3.48-3.43 (m, 1H), 3.37-3.34 (m, 3H), 3.29-3.25 (m, 1H), 3.17-3.11 (m, 1H), 3.09-3.03 (m, 3H), 3.01-2.97 (m, 3H), 2.90-2.78 (m, 2H), 2.57 (d, J=14.8 Hz, 1H), 2.45-2.31 (m, 1H), 2.29-2.11 (m, 2H), 2.05-1.89 (m, 4H), 1.88-1.71 (m, 10H), 1.70-1.57 (m, 2H), 1.45 (d, J=6.0 Hz, 3H), 1.14-0.91 (m, 6H), 0.86 (d, J=6.8 Hz, 3H), 0.44 (s, 3H) ppm.

The compound 10g was prepared in analogy to the preparation of compound 9g by using cis 4-methoxycarbonylcyclohexanecarboxylic acid (compound 10a) instead of trans 4-methoxycarbonylcyclohexanecarboxylic acid (compound 9a).

### Example 11

### cis-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide

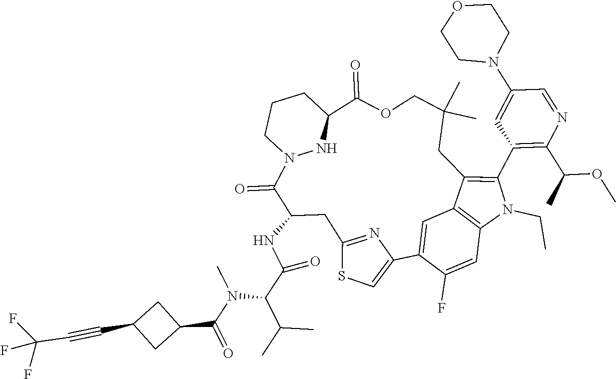

The title compound was prepared in analogy to the preparation of Example 1 by using cis-(2S)-3-methyl-2-[methyl-[3-(3,3,3-trifluoroprop-1-ynyl)cyclobutanecarbonyl]amino]butanoic acid (compound 3B) and (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate G) instead of trans-(2S)-3-methyl-2-[methyl-[3-(3,3,3-trifluoroprop-1-ynyl)cyclobutanecarbonyl]amino]butanoic acid (compound 1I) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 11 (39.6 mg) was obtained as a yellow solid. MS calc'd 1021.5 (MH⁺), measured 1021.5 (MH⁺). ¹H NMR (400 MHz, Methanol-d₄) δ=8.71 (d, J=7.6 Hz, 1H), 8.39 (d, J=2.8 Hz, 1H), 7.88-7.82 (m, 1H), 7.62 (d, J=2.4 Hz, 1H), 7.40-7.32 (m, 1H), 5.84-5.72 (m, 1H), 4.84-4.72 (m, 1H), 4.47-4.34 (m, 2H), 4.32-4.22 (m, 1H), 4.20-4.12 (m, 1H), 4.10-3.98 (m, 1H), 3.87 (t, J=4.8 Hz, 4H), 3.81-3.69 (m, 2H), 3.53-3.36 (m, 9H), 3.29-3.18 (m, 2H), 3.08-2.97 (m, 1H), 2.95-2.87 (m, 3H), 2.83-2.58 (m, 4H), 2.55-2.36 (m, 2H), 2.27-2.12 (m, 2H), 2.00-1.91 (m, 1H), 1.86-1.74 (m, 1H), 1.71-1.56 (m, 1H), 1.46 (d, J=6.4 Hz, 3H), 1.07-0.94 (m, 9H), 0.89-0.82 (m, 3H), 0.68-0.49 (m, 3H) ppm.

### Example 12

### cis-N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide

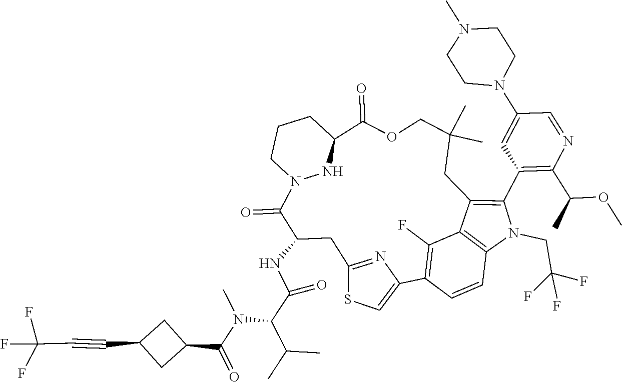

The title compound was prepared in analogy to the preparation of Example 1 by using cis-(2S)-3-methyl-2-[methyl-[3-(3,3,3-trifluoroprop-1-ynyl)cyclobutanecarbonyl]amino]butanoic acid (compound 3B) and (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of trans-(2S)-3-methyl-2-[methyl-[3-(3,3,3-trifluoroprop-1-ynyl)cyclobutanecarbonyl]amino]butanoic acid (compound 1I) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 12 (7.7 mg) was obtained as a white solid. MS calc'd 1088.5 (MH⁺), measured 1088.5 (MH⁺). ¹H NMR (400 MHz, Methanol-d₄) δ=8.54-8.50 (m, 1H), 7.54-7.42 (m, 4H), 5.84-5.96 (m, 1H), 5.20-5.10 (m, 1H), 4.68-4.60 (m, 1H), 4.39 (d, J=12 Hz, 1H), 4.12-3.96 (m, 2H), 3.75-3.60 (m, 3H), 3.57-3.46 (m, 2H), 3.45-3.38 (m, 1H), 3.29-3.23 (m, 5H), 3.22-3.12 (m, 5H), 3.03-3.07 (m, 1H), 3.00 (s, 3H), 2.98-2.95 (m, 3H), 2.93-2.75 (m, 2H), 2.74-2.53 (m, 4H), 2.50-2.34 (m, 2H), 2.25-2.16 (m, 1H), 1.71-1.59 (m, 1H), 1.47-1.43 (m, 3H), 1.38-1.14 (m, 3H), 0.98-0.92 (m, 3H), 0.85-0.78 (m, 6H), 0.76-0.68 (m, 1H), 0.65-0.55 (m, 2H) ppm.

### Example 13

### cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-[2-[5-(trifluoromethyl)pyrimidin-2-yl]ethynyl]cyclobutanecarboxamide

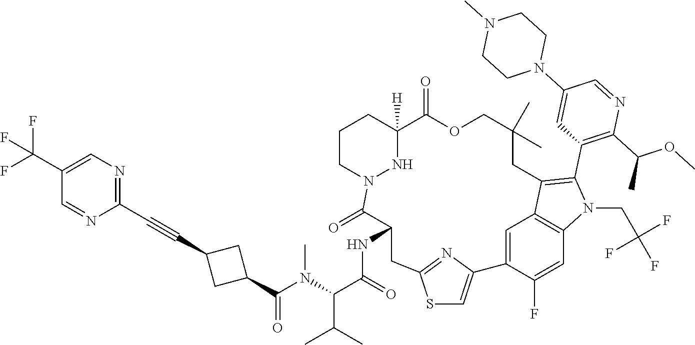

The title compound was prepared in analogy to the preparation of Example 4 by using cis-3-[2-[5-(trifluoromethyl)pyrimidin-2-yl]ethynyl]cyclobutanecarboxylic acid (compound 13B) instead of 4-(3,3,3-trifluoroprop-1-ynyl)benzoic acid (compound 4C). Example 13 (10.8 mg) was obtained as a white solid. MS calc'd 1166.5 (MH⁺), measured 1166.4 (MH⁺). ¹H NMR (400 MHz, Methanol-d₄) δ=9.07 (s, 2H), 8.66 (d, J=7.3 Hz, 1H), 8.49 (d, J=2.9 Hz, 1H), 7.67 (s, 1H), 7.46-7.38 (m, 2H), 5.65-5.61 (m, 1H), 5.15 (br d, J=7.8 Hz, 1H), 4.68-4.56 (m, 1H), 4.46-4.37 (m, 1H), 4.26-4.19 (m, 2H), 3.80-3.67 (m, 3H), 3.61 (q, J=7.2 Hz, 5H), 3.48-3.39 (m, 6H), 3.15 (br d, J=14.7 Hz, 1H), 2.96 (d, J=7.3 Hz, 6H), 2.83-2.76 (m, 2H), 2.70-2.66 (m, 1H), 2.60-2.52 (m, 2H), 2.22 (dt, J=3.2, 7.5 Hz, 2H), 1.99-1.93 (m, 1H), 1.81 (br s, 1H), 1.68-1.60 (m, 1H), 1.45-1.41 (m, 3H), 1.32-1.27 (m, 1H), 1.18 (t, J=6.8 Hz, 4H), 1.01-0.93 (m, 6H), 0.87 (d, J=6.4 Hz, 3H), 0.45-0.40 (s, 3H) ppm.

The compound 13B was prepared in analogy to the preparation of Compound 5B by using cis-tert-butyl 3-ethynylcyclobutanecarboxylate (Intermediate I) and 2-iodo-5-(trifluoromethyl)-pyrimidine instead of cis-tert-butyl (2S)-2-[(3-ethynylcyclobutanecarbonyl)-methyl-amino]-3-methyl-butanoate (compound 1G) and 2-iodopyrimidine.

### Example 14

### N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide

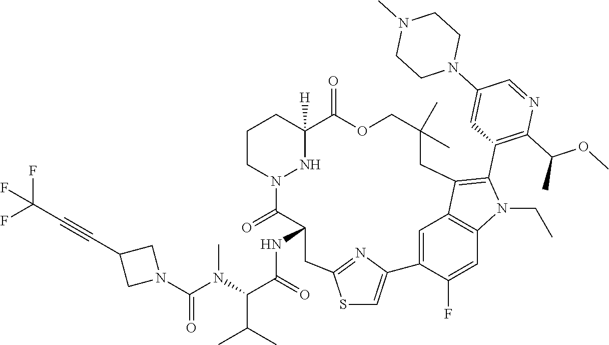

The title compound was prepared in analogy to the preparation of Example 1 by using (2S)-3-methyl-2-[methyl-[3-(3,3,3-trifluoroprop-1-ynyl)azetidine-1-carbonyl]amino]butanoic acid (compound 2E) and (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate D) instead of trans-(2S)-3-methyl-2-[methyl-[3-(3,3,3-trifluoroprop-1-ynyl)cyclobutanecarbonyl]amino]butanoic acid (compound 1I) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 14 (41.2 mg) was obtained as a yellow solid. MS calc'd 1035.5 (MH⁺), measured 1035.5 (MH⁺). ¹H NMR (400 MHz, Methanol-d₄) δ=8.66 (d, J=7.6 Hz, 1H), 8.48 (d, J=2.8 Hz, 1H), 7.65 (d, J=2.4 Hz, 1H), 7.50 (d, J=2.8 Hz, 1H), 7.33 (d, J=12.8 Hz, 1H), 5.79-5.74 (m, 1H), 4.46-4.13 (m, 12H), 4.08-4.03 (m, 2H), 3.77-3.63 (m, 6H), 3.49-3.46 (m, 1H), 3.45-3.43 (m, 1H), 3.09-3.01 (m, 2H), 3.00 (s, 3H), 2.95 (d, J=2.4 Hz, 2H), 2.85 (s, 3H), 2.81-2.74 (m, 1H), 2.64-2.57 (m, 1H), 2.32-2.14 (m, 3H), 1.99-1.92 (m, 1H), 1.86-1.76 (m, 1H), 1.68-1.60 (m, 1H), 1.43 (d, J=6.4 Hz, 3H), 0.98-0.92 (m, 12H), 0.50 (s, 3H).

### Example 15

### N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide

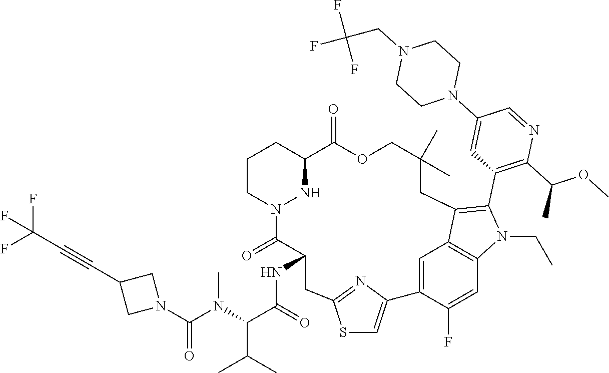

The title compound was prepared in analogy to the preparation of Example 1 by using (2S)-3-methyl-2-[methyl-[3-(3,3,3-trifluoroprop-1-ynyl)azetidine-1-carbonyl]amino]butanoic acid (compound 2E) and (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate F) instead of trans-(2S)-3-methyl-2-[methyl-[3-(3,3,3-trifluoroprop-1-ynyl)cyclobutanecarbonyl]amino]butanoic acid (compound 1I) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 15 (43.2 mg) was obtained as a yellow solid. MS calc'd 1103.5 (MH⁺), measured 1103.5 (MH⁺). ¹H NMR (400 MHz, Methanol-d₄) δ=8.70 (d, J=7.2 Hz, 1H), 8.39-8.36 (m, 1H), 7.80-7.76 (m, 1H), 7.68-7.62 (m, 1H), 7.38-7.32 (m, 1H), 5.85-5.78 (m, 1H), 4.40-4.14 (m, 8H), 4.10-4.02 (m, 2H), 3.78-3.68 (m, 3H), 3.49-3.45 (m, 4H), 3.45-3.42 (s, 1H), 3.19-3.14 (m, 2H), 2.94-2.85 (m, 6H), 2.83 (s, 4H), 2.75-2.69 (m, 1H), 2.22-2.14 (m, 2H), 2.01-1.92 (m, 1H), 1.86-1.75 (m, 1H), 1.69-1.57 (m, 1H), 1.45 (d, J=6.4 Hz, 3H), 1.34-1.23 (m, 1H), 1.05-0.99 (m, 3H), 0.97-0.90 (m, 11H), 0.58 (s, 3H).

### Example 16

### cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-[2-[4-(trifluoromethyl)pyrimidin-2-yl]ethynyl]cyclobutanecarboxamide

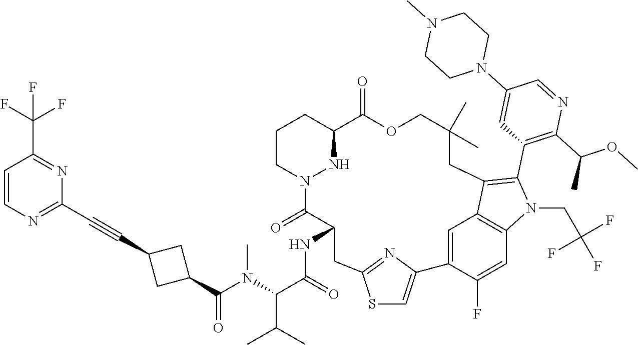

The title compound was prepared in analogy to the preparation of Example 4 by using cis-3-[2-[4-(trifluoromethyl)pyrimidin-2-yl]ethynyl]cyclobutanecarboxylic acid (compound 16B) instead of 4-(3,3,3-trifluoroprop-1-ynyl)benzoic acid (compound 4C). Example 16 (98.2 mg) was obtained as a white solid. MS calc'd 1166.5 (MH⁺), measured 1166.4 (MH⁺). ¹H NMR (400 MHz, Methanol-d₄) δ=9.06-8.99 (m, 1H), 8.71-8.63 (m, 1H), 8.47-8.39 (m, 1H), 7.82-7.73 (m, 1H), 7.71-7.64 (m, 1H), 7.46-7.26 (m, 2H), 5.77-5.60 (m, 1H), 5.20-5.02 (m, 3H), 4.48-4.09 (m, 2H), 3.82-3.74 (m, 1H), 3.72-3.66 (m, 1H), 3.60-3.53 (m, 1H), 3.52-3.46 (m, 1H), 3.46-3.42 (m, 1H), 3.41-3.38 (m, 1H), 3.38-3.34 (m, 4H), 3.17-3.08 (m, 1H), 3.03-2.92 (m, 3H), 2.91-2.84 (m, 1H), 2.83-2.77 (m, 1H), 2.76-2.69 (m, 2H), 2.66-2.60 (m, 5H), 2.60-2.54 (m, 2H), 2.36 (s, 3H), 2.29-2.15 (m, 2H), 2.01-1.90 (m, 1H), 1.86-1.70 (m, 1H), 1.69-1.53 (m, 1H), 1.46-1.38 (m, 3H), 1.32-1.25 (m, 1H), 1.20-1.02 (m, 1H), 1.00-0.94 (m, 5H), 0.92-0.82 (m, 4H), 0.81-0.69 (m, 1H), 0.40 (s, 3H).

The compound 16B was prepared in analogy to the preparation of Compound 5B by using cis-tert-butyl 3-ethynylcyclobutanecarboxylate (Intermediate I) and 2-bromo-4-(trifluoromethyl)pyrimidine instead of cis-tert-butyl (2S)-2-[(3-ethynylcyclobutanecarbonyl)-methyl-amino]-3-methyl-butanoate (compound 1G) and 2-iodopyrimidine.

### Example 18

### N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide

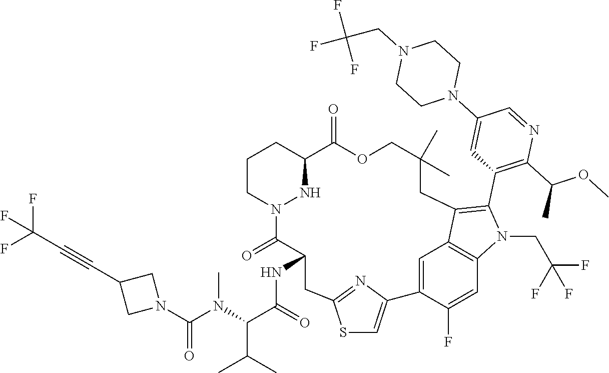

The title compound was prepared in analogy to the preparation of Example 1 by using (2S)-3-methyl-2-[methyl-[3-(3,3,3-trifluoroprop-1-ynyl)azetidine-1-carbonyl]amino]butanoic acid (compound 2E) and (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E) instead of trans-(2S)-3-methyl-2-[methyl-[3-(3,3,3-trifluoroprop-1-ynyl)cyclobutanecarbonyl]amino]butanoic acid (compound 1I) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 18 (24.8 mg) was obtained as a yellow solid. MS calc'd 1157.5 (MH⁺), measured 1157.5 (MH⁺). ¹H NMR (400 MHz, Methanol-d₄) δ=8.71 (d, J=7.2 Hz, 1H), 8.41 (d, J=2.8 Hz, 1H), 7.73-7.64 (m, 2H), 7.48 (d, J=12.4 Hz, 1H), 5.79-5.72 (m, 1H), 5.23-5.16 (m, 1H), 4.44-4.28 (m, 4H), 4.25-3.99 (m, 4H), 3.83-3.73 (m, 1H), 3.72-3.69 (m, 1H), 3.50-3.42 (m, 5H), 3.40-3.36 (s, 3H), 3.25-3.14 (m, 3H), 2.91-2.82 (m, 9H), 2.66 (d, J=14.4 Hz, 1H), 2.24-2.16 (m, 2H), 1.99-1.91 (m, 1H), 1.91-1.68 (m, 1H), 1.68-1.55 (m, 1H), 1.48-1.45 (m, 3H), 0.99-0.91 (m, 11H), 0.51 (s, 3H).

### Example 19

### (2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-isopropyl-4-oxo-4-[3-(3,3,3-trifluoroprop-1-ynyl)azetidin-1-yl]butanamide

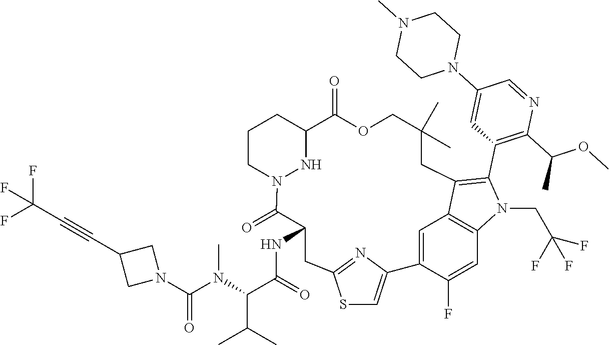

The title compound was prepared in analogy to the preparation of Example 4 by using (2S)-4-tert-butoxy-2-isopropyl-4-oxo-butanoic acid and 3-(3,3,3-trifluoroprop-1-ynyl)azetidine (compound 19B) instead of BOC-N-ME-VAL-OH and 4-(3,3,3-trifluoroprop-1-ynyl)benzoic acid (compound 4C). Example 19 (30.6 mg) was obtained as a white solid. MS calc'd 1074.4 (MH⁺), measured 1074.4 (MH⁺). ¹H NMR (400 MHz, Methanol-d₄) δ=8.67 (d, J=7.6 Hz, 1H), 8.50 (d, J=2.8 Hz 1H), 7.76-7.68 (m, 1H), 7.55 (s, 1H), 7.50-7.44 (m, 1H), 5.78-5.66 (m, 1H), 5.22-5.13 (m, 1H), 4.98-4.89 (m, 2H), 4.57-4.48 (m, 1H), 4.46-4.39 (m, 1H), 4.35-4.27 (m, 1H), 4.26-4.16 (m, 3H), 4.14-4.04 (m, 1H), 4.04-3.99 (m, 1H), 3.98-3.92 (m, 1H), 3.82-3.76 (m, 1H), 3.74-3.62 (m, 3H), 3.59-3.52 (m, 1H), 3.50-3.39 (m, 2H), 3.38-3.33 (m, 4H), 3.21-3.09 (m, 2H), 3.00 (s, 3H), 2.86-2.70 (m, 2H), 2.64-2.44 (m, 2H), 2.26-2.14 (m, 2H), 2.00-1.88 (m, 2H), 1.86-1.73 (m, 1H), 1.70-1.58 (m, 1H), 1.45 (d, J=6.0 Hz, 3H), 1.26-1.06 (m, 1H), 1.03-0.92 (m, 9H), 0.53-0.41 (m, 3H).

The compound 19B was prepared in analogy to the preparation of compound 9g by using tert-butyl 3-ethynylazetidine-1-carboxylate (compound 2A) instead of trans tert-butyl 4-ethynylcyclohexanecarboxylate (compound 9e).

### Example 20

### N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide

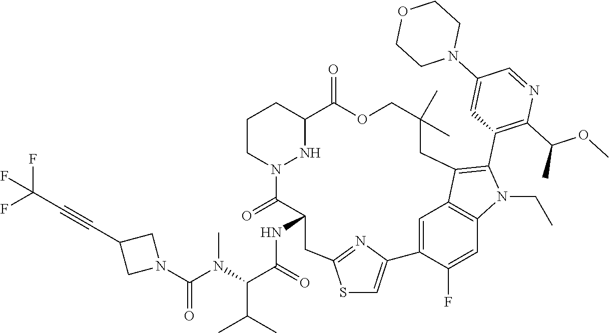

The title compound was prepared in analogy to the preparation of Example 1 by using (2S)-3-methyl-2-[methyl-[3-(3,3,3-trifluoroprop-1-ynyl)azetidine-1-carbonyl]amino]butanoic acid (compound 2E) and (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate G) instead of trans-(2S)-3-methyl-2-[methyl-[3-(3,3,3-trifluoroprop-1-ynyl)cyclobutanecarbonyl]amino]butanoic acid (compound 1I) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 20 (8.8 mg) was obtained as a yellow solid. MS calc'd 1022.5 (MH⁺), measured 1022.5 (MH⁺). ¹H NMR (400 MHz, DMSO-d₆) δ=8.55 (d, J=7.6 Hz, 1H), 8.50-8.39 (m, 2H), 7.67 (d, J=2.4 Hz, 1H), 7.56 (d, J=12.8 Hz, 1H), 7.30 (d, J=1.6 Hz, 1H), 5.53-5.41 (m, 1H), 5.14 (d, J=12.0 Hz, 1H), 4.31-4.21 (m, 4H), 4.21-4.05 (m, 5H), 4.05-3.98 (m, 2H), 3.81-3.70 (m, 6H), 3.57 (s, 2H), 3.26-3.23 (m, 3H), 3.20 (s, 3H), 2.93-2.84 (m, 1H), 2.78-2.68 (m, 4H), 2.11-2.03 (m, 2H), 1.88-1.67 (m, 2H), 1.58-1.45 (m, 1H), 1.33 (d, J=6.0 Hz, 3H), 1.23 (s, 1H), 0.95-0.83 (m, 10H), 0.79 (d, J=6.8 Hz, 3H), 0.37 (s, 3H).

### Example 21

### cis-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-[2-[4-(trifluoromethyl)pyrimidin-2-yl]ethynyl]cyclobutanecarboxamide

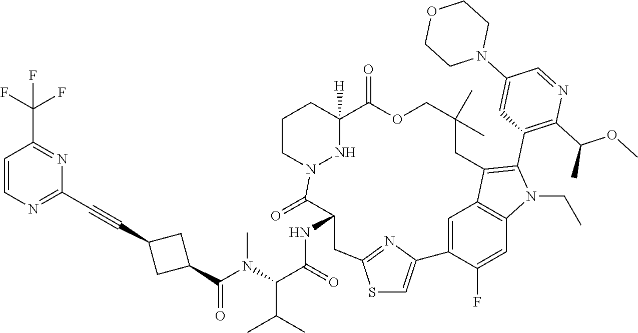

The title compound was prepared in analogy to the preparation of Example 4 by using cis-3-[2-[4-(trifluoromethyl)pyrimidin-2-yl]ethynyl]cyclobutanecarboxylic acid (compound 16B) and (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate G) instead of 4-(3,3,3-trifluoroprop-1-ynyl)benzoic acid (compound 4C) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 21 (22.8 mg) was obtained as a white solid. MS calc'd 1099.5 (MH⁺), measured 1099.5 (MH⁺). ¹H NMR (400 MHz, Methanol-d₄) δ=9.03 (d, J=5.2 Hz, 1H), 8.66-8.61 (m, 1H), 8.40 (d, J=2.8 Hz, 1H), 7.78 (d, J=5.0 Hz, 1H), 7.62 (d, J=2.4 Hz, 1H), 7.32 (d, J=2.8 Hz, 1H), 7.25 (d, J=12.8 Hz, 1H), 5.71 (br d, J=9.0 Hz, 1H), 4.92-4.88 (m, 1H), 4.80-4.76 (m, 1H), 4.47-4.38 (m, 1H), 4.27-4.22 (m, 1H), 4.21-4.16 (m, 2H), 3.88-3.84 (m, 4H), 3.78-3.69 (m, 2H), 3.56-3.48 (m, 1H), 3.46-3.36 (m, 2H), 3.28-3.26 (m, 3H), 3.05-2.92 (m, 5H), 2.86-2.52 (m, 8H), 2.25-2.17 (m, 2H), 1.99-1.92 (m, 1H), 1.88-1.74 (m, 1H), 1.68-1.57 (m, 1H), 1.42 (d, J=6.4 Hz, 3H), 1.34-1.28 (m, 1H), 1.12-1.02 (m, 1H), 0.99-0.84 (m, 12H), 0.53-0.45 (m, 3H).

### Example 22

### cis-N-[(1S)-1-[[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide

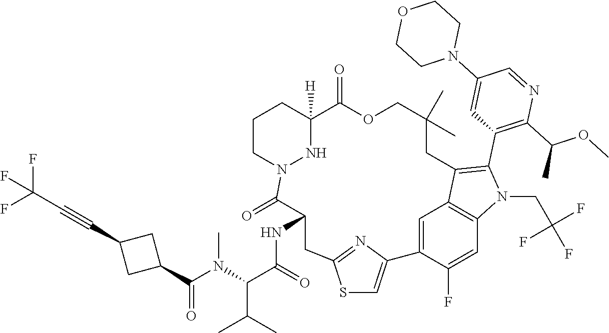

The title compound was prepared in analogy to the preparation of Example 1 by using (2S)-3-methyl-2-[methyl-[3-(3,3,3-trifluoroprop-1-ynyl)azetidine-1-carbonyl]amino]butanoic acid (compound 2E) and (7S,13S)-7-amino-24-fluoro-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate K) instead of trans-(2S)-3-methyl-2-[methyl-[3-(3,3,3-trifluoroprop-1-ynyl)cyclobutanecarbonyl]amino]butanoic acid (compound 1I) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C). Example 22 (41.5 mg) was obtained as a white solid. MS calc'd 1075.4 (MH⁺), measured 1075.4 (MH⁺). ¹H NMR (400 MHz, METHANOL-d₄) δ=8.80-8.63 (d, J=7.2 Hz, 1H), 8.44-8.36 (d, J=2.8 Hz, 1H), 7.71-7.61 (m, 2H), 7.53-7.42 (d, J=12.8 Hz, 1H), 5.76-5.63 (m, 1H), 5.27-5.11 (m, 2H), 4.80-4.72 (m, 1H), 4.49-4.36 (m, 1H), 4.34-4.13 (m, 2H), 3.94-3.84 (m, 4H), 3.83-3.76 (m, 1H), 3.75-3.67 (m, 1H), 3.58-3.43 (m, 2H), 3.39-3.35 (m, 6H), 3.29-3.23 (m, 1H), 3.17-3.09 (m, 1H), 2.98-2.90 (m, 3H), 2.88-2.77 (m, 1H), 2.75-2.60 (m, 3H), 2.56-2.43 (m, 2H), 2.30-2.15 (m, 2H), 2.04-1.91 (m, 1H), 1.90-1.74 (m, 1H), 1.72-1.56 (m, 1H), 1.51-1.43 (d, J=6.0 Hz, 3H), 1.41-1.34 (d, J=6.4 Hz, 1H), 1.22-1.14 (d, J=6.4 Hz, 1H), 1.09-1.03 (d, J=6.4 Hz, 1H), 1.01-0.95 (m, 5H), 0.92-0.80 (m, 3H), 0.55-0.45 (m, 3H).

### BIOLOGICAL EXAMPLE

Compound A168 (page 81 of Table. 1) from WO2021091982 was cited as reference compound for this invention.

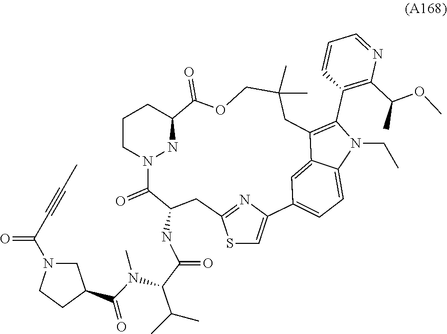

### Example 23

### GSH Reaction Rates

Glutathione (GSH) is a tripeptide found in most of the tissues, especially in high concentrations in the liver, and plays critical roles in protecting cells from oxidative damage and the toxicity of xenobiotic electrophiles, and maintaining redox homeostasis. More specifically, glutathione conjugation helps contribute to detoxification by binding electrophiles that could otherwise bind to proteins or nucleic acids, resulting in cellular damage and genetic mutations.

Many potentially toxic electrophilic xenobiotics and some endogenous compounds are detoxified by conversion to the corresponding glutathione S-conjugate, which consumes inherent GSH and then diminishes detoxification effects. Some drugs and halogenated workplace/environmental contaminants are bioactivated by this mechanism.

On the other hand, conjugation between glutathione and drug molecule in extrahepatic organs as well as in the liver typically leads to the poor PK properties (notably high clearance) of the molecule, and increases its possibility for off-target reactivity (potential liability for various toxicity). Therefore Strategy to minimize the GSH metabolism is very critical. Short T_{1/2} in inherent GSH reaction indicated high GSH reaction rate. Thus T_{1/2} in inherent GSH reaction assay was determined for the screening of candidates.

Reference compounds and compounds of this invention potentially could form conjugation with GSH either through halogenated moieties substitution reaction or direct Michael addition reaction. This test was therefore performed to check GSH reactivity of listed compounds.

For inherent GSH reactivity determination, compounds at 1 μM were incubated at 37° C. with and without 5 mM GSH for 0, 0.5, 1, 2, 4 and 6 h in 100 mM potassium phosphate buffer at pH 7.4. At the end of the designated time points, samples were quenched with acetonitrile containing 10 mM N-ethylmaleimide and an internal standard. Quenched samples were centrifuged, and supernatants were analyzed by LC-MS/MS for compound quantification. If % depletion after 6 hour incubation is less than 20%, compounds were reported as stable; if % depletion is greater than 20%, half-life values are reported.

TABLE 1
GSH Reaction Rates of Examples and
Compounds of present invention

| | | Remaining |
|---|---|---|
| | T½(hour) | at 6 hr in |
| Example | in GSH | GSH (%) |
| A168 | 28.9 | 77 |
| Example 3 | stable | 100 |
| Example 8 | stable | 90 |
| Example 11 | stable | 94 |

Above result clearly shows that reference compounds (A168) formed conjugation with GSH causing its depletion over 6 hours while compounds of current invention maintained the stability with much less or no conjugation with GSH.

### Example 24

### Single Dose Pharmacokinetics (SDPK) Study in Female BALB/c Mice

The purpose of this study was to determine the pharmacokinetics of selected compounds following single intravenous bolus or oral gavage administration in female BALB/c mice. Briefly, two groups of female BALB/c mice (available from Zhejiang Vital River Laboratory Animal Technology Co., Ltd. or Shanghai Lingchang Biotechnology Co., Ltd) (N=3/group) were treated with a single dose of compound intravenously at 3 mg/kg (IV) or orally at 30 mg/kg (PO). Blood samples were collected at 5 min (only for IV), 15 min, 30 min, 1 h, 2 h, 4 h, 7 h and 24 h post-dose. Blood samples were placed on ice until centrifugation to obtain plasma samples. The concentration of compound in plasma samples was determined using LC-MS/MS method. The pharmacokinetic parameters were calculated by non-compartmental analysis.

TABLE 2
Results of SDPK

| | |
|---|---|
| 3 mg/kg, iv | 30 mg/kg, po |

| | CL | Cₘₐₓ | AUC₀₋ₗₐₛₜ |
|---|---|---|---|
| compound | (mL/min/kg) | (ng/mL) | (h*ng/mL) |
| A168 | 77 | 794 | 1583 |
| Example 11 | 14.2 | 1510 | 2176 |

From Table 2, it can be seen that Example 11 has good pharmacokinetic properties in mouse model. Especially Example 11 has the almost 2 folds of Cₘₐₓ, 1.5 folds AUC₀₋ₗₐₛₜ and much lower clearance than A168, which make Example 11 more suitable for treating cancers with KRAS mutation as an orally therapeutic active ingredient in clinic.

### Example 25

### Human Hepatocyte Stability Assay

The hepatocyte stability assay measures the rate of disappearance of a compound from incubations with cryopreserved suspension hepatocytes from human. Positive controls, including Midazolam, Raloxifene and Dextromethorphan, are included in every experiment. Incubations consist of 1 μM tested compound and suspension of human hepatocytes (1×10⁶ cells/mL) in supplemented Williams' E Medium with 10% FBS and 0.5% Penicillin-streptomycin. The hepatocyte suspension was incubated with intermittent shaking 900 rpm at 37° C., in a 5% CO₂ incubator. The reaction was stopped by adding methanol containing internal standard (2 μM Tolbutamide) at 2, 10, 20, 40, 60 and 120 minutes after compound addition, depletion of the parent compound was monitored by LC-MS/MS analysis. For human data, CL_hep (mL/min/kg) >16.24 is high clearance, CL_hep (mL/min/kg)<6.96 is low clearance. 16.24<CL_hep (mL/min/kg) >6.96 is medium clearance.

TABLE 3
Human hepatocytes stability of Examples
and Compounds of present invention

| | CL_hep (Human) | Clearance |
|---|---|---|
| Example | (ml/min/kg) | Category |
| A168 | 8.8 | medium |
| Example 3 | 3.6 | low |
| Example 8 | 2.9 | low |
| Example 11 | 3.3 | low |

Achieving low clearance is advantageous to improve in vivo performance of the compound, such as dose reduction, exposure enhancement, and half-life prolongation. Above result clearly shows that reference compounds (A168) showed medium clearance while compounds of current invention maintained the low clearance in human hepatocytes stability assay.

### Example 26

### Cell Viability Assay

The purpose of this cellular assay was to determine the effects of test compounds on the proliferation of human cancer cell lines NCI-H358 (ATCC-CRL5807) cells, AGS (ATCC-CRL-1739) cells, SW620 (ATCC-CCL-227) over a 3-day treatment period by quantifying the amount of NADPH present at endpoint using Cell Counting Kit-8.

Cells were seeded at 5,000 cells/well (NCI-H358), 2,000 cells/well (AGS) 2,000 cells/well (SW620) in 96-well assay plates (Corning-3699) and incubated overnight. On the day of the assay, diluted compounds were then added in a final concentration of 0.5% DMSO. After 72 hrs incubation, a tenth of the volume of cell counting kit 8 (Dnjindo-CK04) was added into each well. Read the signal (OD450 minus OD650) using EnVision after 2 hrs incubation. IC₅₀ was determined by fitting a 4-parameter sigmoidal concentration response model.

TABLE 4
Activity of Examples and Compounds of present
invention in KRAS Cell viability assay

| | G12C | G12D | G12V |
|---|---|---|---|
| Example | IC50 (μM) | IC50 (μM) | IC50 (μM) |
| A168 | 0.011 | 2.192 | 0.121 |
| Example 1 | 0.027 | >10 | 0.040 |
| Example 2 | 0.002 | 0.020 | 0.010 |
| Example 3 | 0.003 | 3.573 | 2.178 |
| Example 4 | 0.123 | 0.781 | 0.041 |
| Example 5 | 0.007 | 0.536 | 0.230 |
| Example 6 | <0.001 | 2.836 | 4.734 |
| Example 7 | 0.023 | >5 | >5 |
| Example 8 | 0.019 | >5 | >5 |
| Example 9 | 0.436 | >5 | >5 |
| Example 10 | 0.078 | 0.876 | >5 |
| Example 11 | 0.002 | >10 | 1.374 |
| Example 12 | 0.033 | 3.098 | 0.360 |
| Example 13 | 0.006 | 0.328 | 0.036 |
| Example 14 | 0.001 | >10 | 0.007 |
| Example 16 | 0.011 | 0.743 | 0.237 |
| Example 18 | 0.006 | >10 | >10 |
| Example 19 | 0.166 | 3.339 | 0.653 |
| Example 20 | 0.025 | >10 | 0.119 |
| Example 21 | 0.020 | 0.231 | 0.222 |
| Example 22 | 0.012 | >10 | 0.152 |

### Example 27

### KRAS G12C-BRAF NanoBit Assay

This assay is to measure the ability of tested compounds in disruption of the KRAS G12C-BRAF complex at the cellular level, we established the NanoBit cellular assay in mammalian HEK293 (ATCC) cells.

HEK293 cells were grown and maintained using DMEM medium (Thermo Fisher Scientific) with 10% fetal bovine serum and 1% penicillin/streptomycin. Both KRAS G12C and BRAF RBD were cloned into the NanoBit vectors (BiBiT vectors system, Promega) with the orientations SmBit-KRAS G12C and BRAF RBD-LgBit, respectively, and co-transfected into HEK293 cells. Cells were then selected with 100 μg/mL Hygromycin B (10687010, Thermo Fisher) and Blasticidin (5 μg/mL) for 4 weeks to get the stable cell pool.

On the day of the assay, 75 nL of compound solution was presented in a 384-well assay plate as a 16-point 3-fold dilution starting from a final concentration of 30 μM in DMSO. Then cells were seeded at 10,000 cells/25 μL/well in a 384-well plate. After 3 hours of incubation, 6 μL of volume of Nano-Glo® Live Cell Substrate (Promega) was added into each well. Monitor luminescence using ultra384 model in Envision at 20 minutes. Compounds that facilitate disruption of the KRAS G12C-BRAF RBD complex were identified as those eliciting a decrease of luminescence relative to DMSO control wells.

TABLE 5
Activity of Examples and Compounds of present
invention in KRAS G12C-BRAF NanoBit assay

| Example | IC₅₀ (μM) |
|---|---|
| A168 | 0.205 |
| Example 1 | 0.081 |
| Example 2 | 0.018 |
| Example 3 | 0.036 |
| Example 4 | 0.219 |
| Example 5 | 0.106 |
| Example 6 | 0.029 |
| Example 7 | 0.335 |
| Example 8 | 0.054 |
| Example 9 | 0.418 |
| Example 10 | 0.12 |
| Example 11 | 0.012 |
| Example 12 | 0.067 |
| Example 13 | 0.220 |
| Example 14 | 0.036 |
| Example 16 | 0.209 |
| Example 18 | 0.240 |
| Example 19 | 0.075 |
| Example 20 | 0.084 |
| Example 21 | 0.085 |
| Example 22 | 0.036 |

### Example 28

Kras-BRAF with CYPA (500 nM) Interaction Assay

In this example, TR-FRET was also used to measure the compound or compound-CYPA dependent disruption of the KRAS G12C-BRAF complex. This protocol was also used to measure disruption of KRAS G12D or KRAS G12V binding to BRAF by a compound of the invention, respectively. In assay buffer containing 25 mM HEPES PH=7.4 (4-(2-hydroxyethyl)-1-piperazineethanesulfonic acid, Thermo, 15630080), 0.002% Tween20, 0.1% BSA, 100 mM NaCl, 5 mM MgCl₂, 10 μM GMPPNP (Guanosine 5′-[β,γ-imido]triphosphate trisodium salt hydrate, Sigma, G0635), tagless CYPA, GMPPNP loaded 6His-KRAS proteins, and GST-BRAF^{RBD} were mixed in a well of a 384-well assay plate at final concentrations of 50 nM, 6.25 nM and 1 nM, respectively. Compound was present in plate wells as a 16-point 3-fold dilution series starting at a final concentration of 10 μM and incubated for 3 hours. A mixture of MAb Anti-6His-XL665 (Cisbio, 61HISXLB) and Mab anti-GST-TB cryptate (Cisbio, 61GSTTLB) was then added at a final concentration of 6.67 nM and 0.21 nM, respectively, and the plate was incubated for an additional 1.5 hours. TR-FRET signal was read on a PHERstar FSX microplate reader (Ex320 nm, Em 665/615 nm). Compounds that facilitate disruption of the KRAS-BRAF complex were identified as those eliciting a decrease in the TR-FRET ratio relative to DMSO control wells.

TABLE 6
Activity of Examples and Compounds of present invention
in KRAS-BRAF with CYPA (500 nM) interaction assay

| | G12C | G12D | G12V |
|---|---|---|---|
| Example | IC₅₀ (μM) | IC₅₀ (μM) | IC₅₀ (μM) |
| A168 | 0.173 | >10 | 3.618 |
| Example 1 | 3.353 | >10 | >10 |
| Example 2 | 1.788 | >10 | 6.740 |
| Example 3 | 0.099 | >10 | >10 |
| Example 4 | 0.430 | 0.142 | >10 |
| Example 5 | 0.128 | 9.945 | 1.101 |
| Example 6 | 0.021 | >10 | >10 |
| Example 7 | >10 | >10 | >10 |
| Example 8 | 0.118 | >10 | >10 |
| Example 9 | 0.819 | 3.015 | 1.979 |
| Example 10 | >10 | 1.911 | 1.485 |
| Example 11 | 0.065 | >10 | >10 |
| Example 12 | 0.063 | 1.466 | 0.484 |
| Example 13 | 0.065 | >10 | >10 |
| Example 14 | 1.665 | 4.296 | 1.884 |
| Example 19 | 0.240 | >10 | 2.252 |

### Example 29

pERK Inhibition Assay

This assay is to measure the ability of test compounds in inhibiting the phosphorylation of ERK, the downstream signaling of KRAS G12C in NCI-H358 cells, KRAS G12D in AGS cells, and KRAS G12V in SW620. NCI-H358 (ATCC-CRL5807) cells, AGS (ATCC-CRL-1739) cells, SW620 (ATCC-CCL-227) cells were all grown and maintained using RPMI-1640 medium (Thermo Fisher Scientific) with 10% fetal bovine serum and 1% penicillin/streptomycin. On the day prior to compound addition, cells were plated in tissue culture-treated 96 well plates (Corning-3699) at a density of 30,000 cell/well, 20,000 cell/well, 30,000 cell/well for NCI-H358, AGS and SW620 respectively, and allowed for attachment overnight. Diluted compounds were then added in a final concentration of 0.5% DMSO. After 4 hours of incubation, the medium was removed, 100 μL of 4% formaldehyde was added, and the assay plates were incubated at room temperature for 20 minutes. The plates were then washed once with phosphate buffered saline (PBS), and permeabilized with 100 μL of chilled methanol for 10 minutes. Non-specific antibody binding to the plates was blocked using 50 μL 1×BSA blocking buffer (Thermo-37520, 10-fold dilution by Phosphate-Buffered Saline Tween (PBST) for at least 1 hour at room temperature.

The amount of phosphor-ERK was determined using an antibody specific for phosphorylated form of ERK. Primary antibody (pERK, CST-4370, Cell Signaling Technology) was diluted 1:300 in blocking buffer, with 50 μL aliquoted to each well, and incubated overnight at 4° C. Cells was washed five times for 5 minutes with PBST. Secondary antibody (HRP-linked anti-rabbit IgG, CST-7074, Cell Signaling Technology) was diluted 1:1000 in blocking buffer, and 50 μL was added to each well and incubated 1-2 hrs at room temperature. Cells was washed 5 times for 5 minutes with PBST, 100 μL TMB ELISA substrate (abcam-ab171523) were added and gently shake for 20 minutes. 50 μL stop solution (abcam-ab171529) were added, and then read the signal (OD450) by EnVision.

IC₅₀ was determined by fitting a 4-parameter sigmoidal concentration response model.

TABLE 7
Activity of Examples and Compounds of present
invention in KRAS pERK inhibition assay

| | G12C | G12D | G12V |
|---|---|---|---|
| Example | IC₅₀ (μM) | IC₅₀ (μM) | IC₅₀ (μM) |
| A168 | 0.053 | N.A. | 0.176 |
| Example 1 | 0.035 | 1.100 | 0.063 |
| Example 2 | 0.013 | N.A. | 0.007 |
| Example 3 | 0.007 | 0.378 | 0.009 |
| Example 4 | 0.063 | 0.509 | 0.006 |
| Example 5 | 0.058 | 0.145 | <0.001 |
| Example 6 | 0.002 | N.A. | 0.008 |
| Example 7 | 1.102 | N.A. | 0.099 |
| Example 8 | 0.005 | >10 | 0.125 |
| Example 9 | 0.292 | 1.766 | 0.498 |
| Example 10 | 0.048 | 0.152 | 0.065 |
| Example 11 | 0.011 | 3.768 | 0.187 |
| Example 12 | 0.092 | 0.667 | 0.201 |
| Example 13 | 0.045 | 1.809 | 0.056 |
| Example 14 | 0.005 | 0.020 | 0.003 |
| Example 15 | 0.020 | >10 | 0.021 |
| Example 16 | 0.021 | >10 | 0.031 |
| Example 18 | 0.051 | >10 | 0.048 |
| Example 19 | 0.221 | >10 | 0.198 |
| Example 20 | 0.093 | 2.512 | 0.096 |
| Example 21 | 0.019 | >10 | 0.076 |
| Example 22 | 0.013 | 0.172 | 0.078 |

### Example 30

### Stable KRAS Mutant Cell Fines and Cell Viability Assay.

The aim of the study was to determine the potency and efficacy of compounds for cell proliferation using CellTiter-Glo® (CTG) Luminescent Cell Viability Assay (Promega Corp., Madison, WI). We cloned 14 KRAS^{G12C} variant sequences with secondary mutations (V8A, V9Y, S17E, T58I, A59T, S65W, R68S, D69P, M72I, D92R, H95N, Y96D, Q99F, Q99W, Y96H, and F156L) into the Miapaca-2. Totally 14 stable Miapaca-2 mutant cell lines were established through lentivirus infection. For the cell viability assay, cells were dosed with compounds in a 9-point dose-response using a 4 fold dilution series at a top dose of 10 μM. KRAS mutant cells were maintained in DMEM+10% FBS+2.5% HI Horse serum+1% PS+1 μg/mL Puromycin and seeded into 96-well plates at 800-1,500 cells per well 24 h before compound addition and then incubated with compound for 3 d before assaying viability (CellTiter-Glo, Promega). Assays were performed in biological duplicates. Nonlinear regression curves were fitted using Xfit. IC50 (absolute IC50) is the dose at which the estimated viability is 50% relative to untreated wells. Inhibition rate of the compound is calculated according to the formula below: % inhibition=100−100×(Luminescence value−HPE)/(ZPE−HPE).
HPE: Luminescence value from the wells with only medium
ZPE: Luminescence value from the wells with DMSO

TABLE 8
cell viability (IC50 (μM))in mutant cells with KRAS G12C and other mutations

| | G12C + | G12C + | G12C + | G12C + | G12C + | G12C + | G12C + | G12C + | G12C + | G12C + | G12C + | G12C + | G12C + | G12C + |
|---|---|---|---|---|---|---|---|---|---|---|---|---|---|---|
| | V8A | V9Y | S17E | A59T | T58I | D60P | S65W | R68S | M72I | D92R | H95N | Y96D | Q99W | F156L |
| Example | 0.001 | 0.003 | 0.003 | 0.032 | 0.003 | 0.097 | 0.003 | 0.022 | 0.001 | 0.002 | 0.003 | 0.006 | 0.003 | 0.003 |
| 11 | | | | | | | | | | | | | | |

1. A compound of formula (I), 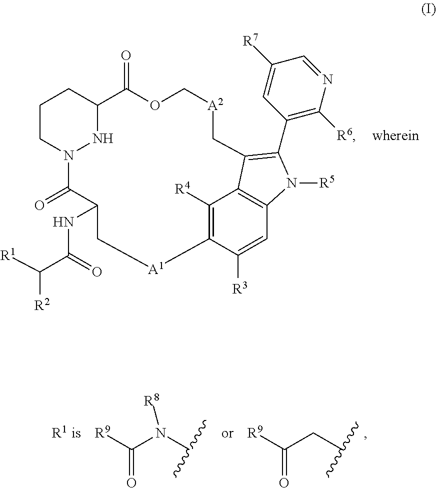
wherein R⁸ is C₁₋₆alkyl;
R⁹ is C₃₋₇cycloalkyl, azetidinyl or phenyl, said C₃₋₇cycloalkyl, azetidinyl and phenyl being substituted by haloC₃₋₆alkynyl, (haloC₃₋₆alkylpyrimidinyl)C₂₋₆alkynyl or pyrimidinylC₂₋₆alkynyl;
R² is C₁₋₆alkyl;
R³ is H or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene;
A² is C₁₋₆alkylene;
with the proviso that R³ and R⁴ are not H simultaneously;
or a pharmaceutically acceptable salt thereof.

2. A compound of formula (Ia), 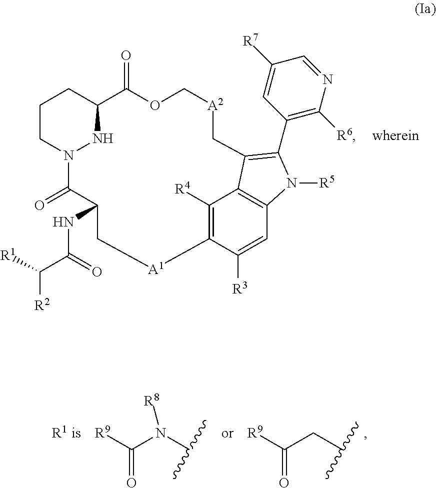
wherein R⁸ is C₁₋₆alkyl;
R⁹ is C₃₋₇cycloalkyl, azetidinyl or phenyl, said C₃₋₇cycloalkyl, azetidinyl and phenyl being substituted by haloC₃₋₆alkynyl, (haloC₃₋₆alkylpyrimidinyl)C₂₋₆alkynyl or pyrimidinylC₂₋₆alkynyl;
R² is C₁₋₆alkyl;
R³ is H or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene;
A² is C₁₋₆alkylene;
with the proviso that R³ and R⁴ are not H simultaneously;
or a pharmaceutically acceptable salt thereof.

3. A compound according to claim 1 or 2, wherein R¹ is 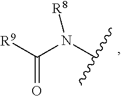
wherein R⁸ is C₁₋₆alkyl; R⁹ is C₃₋₇cycloalkyl substituted by haloC₃₋₆alkynyl.

4. A compound according to claim 1 or 2, wherein R¹ is 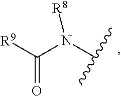
wherein R⁸ is methyl; R⁹ is cyclobutyl substituted by 3,3,3-trifluoroprop-1-ynyl.

5. A compound according to any one of claims 1-4, wherein R⁹ is 3-(3,3,3-trifluoroprop-1-ynyl)cyclobutyl.

6. A compound according to any one of claims 1-5, wherein R² is isopropyl.

7. A compound according to any one of claims 1-6, wherein R³ is halogen.

8. A compound according to any one of claims 1-7, wherein R³ is fluoro.

9. A compound according to any one of claims 1-8, wherein R⁴ is H or fluoro.

10. A compound according to any one of claims 1-9, wherein R⁴ is H.

11. A compound according to any one of claims 1-10, wherein R⁵ is ethyl or 2,2,2-trifluoroethyl.

12. A compound according to any one of claims 1-11, wherein R⁶ is 1-methoxyethyl.

13. A compound according to any one of claims 1-12, wherein R⁷ is morpholinyl, 4-(2,2,2-trifluoroethyl)piperazin-1-yl or 4-methylpiperazin-1-yl.

14. A compound according to any one of claims 1-13, wherein A¹ is 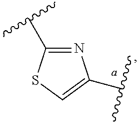 [SMILES: *c1csc(*)n1]
wherein bond “a” connects to indole ring.

15. A compound according to any one of claims 1-14, wherein A² is dimethylmethylene.

16. A compound according to claim 1 or 2, wherein
R¹ is 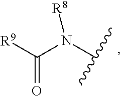
wherein R⁸ is C₁₋₆alkyl; R⁹ is C₃₋₇cycloalkyl substituted by haloC₃₋₆alkynyl;
R² is C₁₋₆alkyl;
R³ is halogen;
R⁴ is H;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is 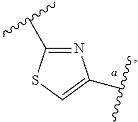 [SMILES: *c1csc(*)n1]
wherein bond “a” connects to indole ring;
A² is C₁₋₆alkylene;
or a pharmaceutically acceptable salt thereof.

17. A compound according to claim 16, wherein
R¹ is 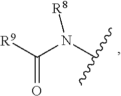
wherein R⁸ is methyl; R⁹ is 3-(3,3,3-trifluoroprop-1-ynyl)cyclobutyl;
R² is isopropyl;
R³ is fluoro;
R⁴ is H;
R⁵ is ethyl or 2,2,2-trifluoroethyl;
R⁶ is (1S)-1-methoxyethyl;
R⁷ is morpholinyl, 4-(2,2,2-trifluoroethyl)piperazin-1-yl or 4-methylpiperazin-1-yl;
A¹ is 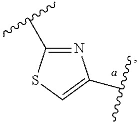 [SMILES: *c1csc(*)n1]
wherein bond “a” connects to indole ring;
A² is dimethylmethylene;
or a pharmaceutically acceptable salt thereof.

18. A compound selected from:
trans-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide;
cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)benzamide;
cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(2-pyrimidin-2-ylethynyl)cyclobutanecarboxamide;
cis-N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
cis-N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
cis-N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
trans-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)cyclohexanecarboxamide;
cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-4-(3,3,3-trifluoroprop-1-ynyl)cyclohexanecarboxamide;
cis-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
cis-N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-[2-[5-(trifluoromethyl)pyrimidin-2-yl]ethynyl]cyclobutanecarboxamide;
N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide;
N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide;
cis-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-[2-[4-(trifluoromethyl)pyrimidin-2-yl]ethynyl]cyclobutanecarboxamide;
N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide;
(2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-isopropyl-4-oxo-4-[3-(3,3,3-trifluoroprop-1-ynyl)azetidin-1-yl]butanamide;
N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)azetidine-1-carboxamide;
cis-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-[2-[4-(trifluoromethyl)pyrimidin-2-yl]ethynyl]cyclobutanecarboxamide; and
cis-N-[(1S)-1-[[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-3-(3,3,3-trifluoroprop-1-ynyl)cyclobutanecarboxamide;
or a pharmaceutically acceptable salt thereof.

19. A process for the preparation of a compound according to any one of claims 1 to 18 comprising any of the following steps:
a) coupling reaction between compound of formula (II), 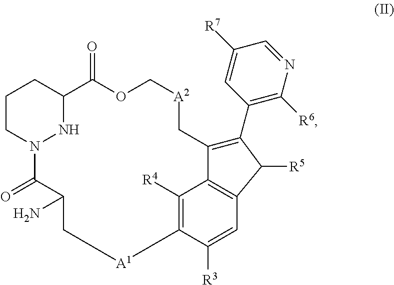
and acid (III), 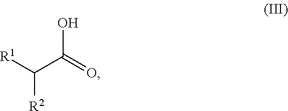
in the presence of a coupling reagent and a base to form the compound of formula (I);
wherein R¹, R², R³, R⁴, R⁵, R⁶, R⁷, A¹ and A² are defined as in any one of claims 1 to 17; the coupling reagent is T₃P, HATU, PyBOP or EDCI/HOBt; the base is TEA, DIEPA or DMAP.

20. A compound or pharmaceutically acceptable salt according to any one of claims 1 to 18 for use as therapeutically active substance.

21. A pharmaceutical composition comprising a compound in accordance with any one of claims 1 to 18 and a pharmaceutically acceptable excipient.

22. The use of a compound according to any one of claims 1 to 18 for treating a KRAS G12C protein-related disease.

23. The use of a compound according to any one of claims 1 to 18 for treating a KRAS G12C, G12D and G12V protein-related disease.

24. The use of a compound according to any one of claims 1 to 18 for inhibiting RAS interaction with downstream effectors, wherein the downstream effectors are RAF and PI3K.

25. The use of a compound according to any one of claims 1 to 18 for inhibiting the propagating oncogenic MAPK and PI3K signaling.

26. The use of a compound according to any one of claims 1 to 18 for the treatment or prophylaxis of KRAS mutation driven cancers, wherein the cancer is selected from pancreatic cancer, colorectal cancer, lung cancer, esophageal cancer, gallbladder cancer, melanoma ovarian cancer and endometrial cancer.

27. The use of a compound according to any one of claims 1 to 18 for the treatment or prophylaxis of KRAS mutation driven cancers, wherein the cancer is selected from pancreatic adenocarcinoma, colorectal cancer and non-small cell lung cancer.

28. A compound or pharmaceutically acceptable salt according to any one of claims 1 to 18 for the treatment or prophylaxis of KRAS mutation driven cancers, wherein the cancer is selected from pancreatic adenocarcinoma, colorectal cancer and non-small cell lung cancer.

29. The use a compound according to any one of claims 1 to 18 for the treatment or prophylaxis of KRAS mutation driven cancers, wherein the cancer comprises a first mutation that is G12C, and a second mutation at a position selected from V8A, V9Y, S17E, T58I, A59T, S65W, R68S, D69P, M72I, D92R, H95N, Y96D, Q99F, Q99W, Y96H, and F156L.

30. The use of a compound according to any one of claims 1 to 18 for the preparation of a medicament for the treatment or prophylaxis of KRAS mutation driven cancers, wherein the cancer is selected from pancreatic adenocarcinoma, colorectal cancer and non-small cell lung cancer.

31. The use of a compound according to any one of claims 1 to 18 for the preparation of a medicament for the treatment or prophylaxis of KRAS mutation driven cancers, wherein the cancer comprises a first mutation that is G12C, and a second mutation at a position selected from V8A, V9Y, S17E, T58I, A59T, S65W, R68S, D69P, M72I, D92R, H95N, Y96D, Q99F, Q99W, Y96H, and F156L.

32. A method for the treatment or prophylaxis of KRAS mutation driven cancers, wherein the cancer is selected from pancreatic adenocarcinoma, colorectal cancer and non-small cell lung cancer, which method comprises administering a therapeutically effective amount of a compound as defined in any one of claims 1 to 18.

33. A method for the treatment or prophylaxis of KRAS mutation driven cancers, wherein the cancer comprises a first mutation that is G12C, and a second mutation at a position selected from V8A, V9Y, S17E, T58I, A59T, S65W, R68S, D69P, M72I, D92R, H95N, Y96D, Q99F, Q99W, Y96H, and F156L.

34. A compound or pharmaceutically acceptable salt according to any one of claims 1 to 18, when manufactured according to a process of claim 19.

35. The invention as hereinbefore described.
